(19) United States
(12) Patent Application Publication
(10) Pub. No.: US 2014/0213772 A1
(43) Pub. Date: Jul. 31, 2014

(54) CROSS-OVER DUAL VARIABLE DOMAIN IMMUNOGLOBULIN CONSTRUCTS

(71) Applicant: AbbVie, Inc., Worcester, MA (US)

(72) Inventors: Tariq GHAYUR, Holliston, MA (US); Jijie GU, Shrewsbury, MA (US)

(73) Assignee: AbbVie, Inc., Worcester, MA (US)

(21) Appl. No.: 14/141,504

(22) Filed: Dec. 27, 2013

Related U.S. Application Data
(60) Provisional application No. 61/746,619, filed on Dec. 28, 2012.

(51) Int. Cl. C07K 16/46

(52) U.S. Cl. CPC C07K 16/468
USPC 530/387.3

(57) ABSTRACT

Engineered cross-over DVD-Ig binding proteins that bind to two or more target proteins (e.g., antigens) are provided, along with methods of making and uses in the prevention, diagnosis, and/or treatment of disease.

### RELATED APPLICATIONS

This application claims priority to US Provisional Patent Application U.S. Ser. No. 61/746,619, filed Dec. 28, 2012 entitled “Cross-Over Dual Variable Domain Immunoglobulin (coDVD-Ig) Constructs,” which is incorporated herein by reference in its entirety.

### FIELD OF THE INVENTION

This disclosure relates to dual-variable-domain (DVD) binding proteins. More particularly, the disclosure relates to modified DVD-Ig having cross-over variable domains

### BACKGROUND

Target-binding proteins that possess preferable pharmacodynamic and pharmcokenetic features have attracted more and more attention in the development of biologic therapeutics. Substantial amount of efforts has been dedicated to the optimization of the amino acid sequences of immunoglobulin (e.g., antibody amino acid sequences) in order to obtain immunoglobulins have superior therapeutic effects. These modified immunoglobulins may have different structures and properties from those found in naturally existing immunoglobulins. These modified structures and properties may lead to the superior therapeutic effects achieved by these immunoglobulins.

An immunoglobulin is an ideal platform for drug development because of its various desirable intrinsic properties. For instance, immunoglobulins typically have great target specificity, superior biostability and bioavailability, less toxicity, and sufficient target binding affinity to maximize therapeutic effects.

Multi-specific (including bi-specific) antibodies combine specificities of two or more mAbs in a single agent. The result is that increased efficacy or novel activity may be achieved by dual/multiple targeting. In the context of an antibody drug, for example, a multi-specific antibody may be easier to characterize and may help reduce development and/or production costs as compared to multiple individual agents.

Multi-specific antibodies have broad therapeutic and diagnostic uses. For instance, bi-specific antibodies (bsAbs) may offer novel opportunities and applications which may be difficult to achieve using single agent combinations. Potential applications for bsAbs may include, for example: (1) additive and synergistic effects by targeting distinct disease mechanisms; (2) novel receptor modulation by targeting two epitopes on the same receptor or two different receptors on the same cell (see section 3 below); (3) tissue or site specific targeting and transport of therapeutics to or through privileged sites (brain, intracellularly, etc) using molecular Trojan horse strategy; (4) re-directed cytotoxicity by bringing various immune effector cells in proximity to tumors; (5) improving specificity by utilizing avidity; (6) efficient clearance of toxins, immune complexes and pathogens, and (7) imaging and diagnostics. Other aspects of bispecific binding proteins have been detailed in recent reviews. See, e.g., Choi et al., 2011; Fagete and Fischer, 2012; Fischer and Leger, 2007; Gu and Ghayur, 2010; and Kontermann, 2012).

U.S. Pat. Nos. 8,258,268 and 7,612,181 provide a novel family of binding proteins capable of binding two or more antigens with high affinity, called the dual variable domain binding protein (DVD binding protein) or Dual Variable Domain Immunoglobulin (DVD-Ig™) construct.

Certain DVD-Ig™ molecules show reduced affinity to its inner domain antigen. Depending on the sequence and the nature of the antigen recognized by inner antigen binding domain, the inner binding domain may display a reduced affinity to its antigen (i.e., loss of on-rate in comparison to the parental antibody). One possible explanation for this observation is that the outer antigen binding domain may poise steric hindrance on the inner antigen binding domain and makes the inner antigen binding domain somewhat inaccessible for its antigens, especially for antigens with larger dynamic size or membrane molecules.

Described here for the first time is a functional extension of the DVD-Ig™ construct, wherein the variable binding domains of the DVD-Ig™ construct are not constrained to a specific locus along the polypeptide chains of a DVD-Ig™ dimer or tetramer construct, thereby creating a DVD-Ig™ construct that is capable of establishing a “cross-over” conformation with its associated dimer (or tetramer) polypeptide chain partner in a unique manner to form a functional binding protein. Such DVD-Ig™ constructs capable of forming such cross-over conformations to create a functional binding protein are referred to as “cross-over DVD-Ig™” constructs, or “coDVD-Ig™” constructs.

### SUMMARY

This disclosure advances the art by providing a number of multi-specific binding proteins capable of binding at least two proteins (e.g., antigens). More specifically, cross-over dual-variable-domain (DVD) Igs are disclosed which are generated by crossing over light chain and the heavy chain variable domains of a dual-variable-domain (DVD) Ig or Ig like protein. The resulting proteins are called “cross-over DVD-Ig™” (“coDVD-Ig™)” constructs, or VD (variable domain) cross-over binding protein in this disclosure.

In one aspect, the cross-over of variable domains may help resolve the issue of affinity loss in the inner antigen-binding domains of some DVD-Ig molecules. In another aspect, the length and sequence of the linkers linking the variable domains may be optimized for each format and antibody sequence/structure (frameworks) to achieve desirable properties. The disclosed concept and methodology may also be extended to Ig or Ig like proteins having more than two antigen binding domains. More than a dozen different formats of bi-specific cross-over DVD Igs are disclosed. The design of the vectors, and methods of constructing the vectors, and methods for expressing, purifying and characterizing the proteins are also disclosed.

In one aspect, similar to one of the representative DVD-Ig molecules disclosed in U.S. Pat. No. 7,612,181, a binding protein of the present disclosure may have the following format (also referred to as “format 1”): VL1-linker-VL2-CL and VH1-linker-VH2-CH1-Fc. In one aspect, the first polypeptide chain may contain a structure represented by the formula VL1-(X1)n-VL2-CL-(X2)n, wherein VL1 is a first light chain variable domain, VL2 is a second light chain variable domain, CL is a light chain constant domain, X1 is a linker with the proviso that it is not a constant domain, and X2 does not comprise an Fc region; and the second polypeptide chain may comprise a structure represented by the formula VH1-(X1)n-VH2-CH-(X2)n, wherein VH1 is a first heavy chain variable domain, VH2 is a second heavy chain variable domain, CH is a heavy chain constant domain, X1 is a linker with the proviso that it is not a constant domain, and X2 is an Fc region; wherein n is 0 or 1, and the VL1 and VH1 (also referred to as “VD1” and collectively as “VD1s”) domains on the first and second polypeptide chains form one functional binding site for antigen A, and wherein the VL2 and VH2 domains (also referred to as “VD2” and collectively as “VD2s”) on the first and second polypeptide chains form one functional binding site for antigen B. In certain embodiments, this binding protein is capable of binding both antigens A and B simultaneously.

In Format 1, the VD1s from different polypeptide chains form an N-terminal (outer) antigen binding domain, while VD2s from different polypeptide chains form a C-terminal (inner) antigen binding domain. The two functional antigen binding domains are in series connection. Some DVD-Ig molecules in format 1 show a position effect on the inner antigen binding domain Depending on the sequence and the nature of the antigen recognized by inner antigen binding domain, this binding domain may display a reduced affinity to its antigen (i.e., loss of on-rate in comparison to the parental antibody). One possible explanation for this observation is that the outer antigen binding domain may poise steric hindrance on the inner antigen binding domain and makes the inner antigen binding domain somewhat inaccessible for its antigens, especially for antigens with larger dynamic size or membrane molecules.

In some of the conformations observed by TEM, outer antigen binding domain poised steric hindrance on inner antigen binding domain. In format 1 DVD-Ig™ even at the most open conformation for the inner antigen-binding domain, the 3-D space available for antigen-binding of the inner antigen binding domain is still restricted to a certain limit, this could reduce inner antigen binding domain affinity, especially the on-rate, when the inner domain antigen has larger size or is a membrane-bound protein. To solve this problem, modified binding proteins may be designed such that the variable domains may be crossed over to form two antigen binding domains that are both more fully exposed to target antigens as compared to those of the parental antibodies.

In one aspect, the instant disclosure provides a binding protein (also referred to as “format 2”) is disclosed which contains at least two polypeptide chains, namely, first and second polypeptide chains. In certain embodiments, the first polypeptide chain may contain a structure represented by the formula VL1-(X1)n-VL2-(X2)n-CL-(X3)n, wherein VL1 is a first light chain variable domain, VL2 is a second light chain variable domain, CL is a light chain constant domain, X1 and X2 are linkers with the proviso that they are not a constant domain, X3 may comprise an Fc region, and n may be 0 or 1. In certain embodiments, the second polypeptide chain may contain a structure represented by the formula VH2-(X4)n-VH1-(X5)n-CH-(X6)n, wherein VH1 is a first heavy chain variable domain, VH2 is a second heavy chain variable domain, CH is a heavy chain constant domain, X4 and X5 are linkers with the proviso that they are not a constant domain, X6 may comprise an Fc region with the proviso that if X3 contains an Fc region, then X6 does not contain an Fc region, and n may be 0 or 1. In certain embodiments, n may be 0 or 1, and the VL1 and VH1 domains form one functional binding site for antigen A, while the VL2 and VH2 domains form one functional binding site for antigen B. In certain embodiments, the binding protein is capable of binding both antigens A and B simultaneously.

In another aspect, a binding protein (also referred to as “format 3”) is disclosed which contains at least two polypeptide chains, namely, first and second polypeptide chains. In certain embodiments, the first polypeptide chain may contain a structure represented by the formula VL1-(X1)n-VH2-(X2)n-CL-(X3)n, wherein VL1 is a first light chain variable domain, VH2 is a second heavy chain variable domain, CL is a light chain constant domain, X1 and X2 are linkers with the proviso that they are not a constant domain, X3 may comprise an Fc region, and n may be 0 or 1. In certain embodiments, the second polypeptide chain may contain a structure represented by the formula VH1-(X4)n-VL2-(X5)n-CH-(X6)n, wherein VH1 is a first heavy chain variable domain, VL2 is a second light chain variable domain, CH is a heavy chain constant domain, X4 and X5 are linkers with the proviso that they are not a constant domain, X6 may comprise an Fc region with the proviso that if X3 contains an Fc region, then X6 does not contain an Fc region, and n may be 0 or 1. In certain embodiments, the VL1 and VH1 domains may form one functional binding site for antigen A, while the VL2 and VH2 domains may form one functional binding site for antigen B. In certain embodiments, the binding protein is capable of binding both antigens A and B simultaneously.

In another aspect, a binding protein (also referred to as “format 4”) is disclosed which contains at least two polypeptide chains, namely, first and second polypeptide chains. In certain embodiments, the first polypeptide chain may contain a structure represented by the formula VH1-(X1)n-VL2-(X2)n CL-(X3)n, wherein VH1 is a first heavy chain variable domain, VL2 is a second light chain variable domain, CL is a light chain constant domain, X1 and X2 are linkers with the proviso that they are not a constant domain, X3 may comprise an Fc region, and n may be 0 or 1. In certain embodiments, the second polypeptide chain may contain a structure represented by the formula VL1-(X4)n-(X5)n VH2-CH-(X6)n, wherein VL1 is a first light chain variable domain, VH2 is a second heavy chain variable domain, CH is a heavy chain constant domain, X4 and X5 are linkers with the proviso that they are not a constant domain, X6 may comprise an Fc region with the proviso that if X3 contains an Fc region, then X6 does not contain an Fc region, and n may be 0 or 1. In certain embodiments, the VL1 and VH1 domains may form one functional binding site for antigen A, while the VL2 and VH2 domains may form one functional binding site for antigen B. In certain embodiments, the binding protein is capable of binding both antigens A and B simultaneously.

In another aspect, a binding protein (also referred to as “format 5”) is disclosed which contains at least two polypeptide chains, namely, first and second polypeptide chains. In certain embodiments, the first polypeptide chain may contain a structure represented by the formula VH1-(X1)n VH2-(X2)n-CL-(X3)n, wherein VH1 is a first heavy chain variable domain, VH2 is a second heavy chain variable domain, CL is a light chain constant domain, X1 and X2 are linkers with the proviso that they are not a constant domain, X3 may comprise an Fc region, and n may be 0 or 1. In certain embodiments, the second polypeptide chain may contain a structure represented by the formula VL1-(X4)n-VL2-(X5)n-CH-(X6)n, wherein VL1 is a first light chain variable domain, VL2 is a second light chain variable domain, CH is a heavy chain constant domain, X4 and X5 are linkers with the proviso that they are not a constant domain, X6 may comprise an Fc region with the proviso that if X3 contains an Fc region, then X6 does not contain an Fc region, and n may be 0 or 1. In certain embodiments, the VL1 and VH1 domains may form one functional binding site for antigen A, while the VL2 and VH2 domains may form one functional binding site for antigen B. In certain embodiments, the binding protein is capable of binding both antigens A and B simultaneously.

In another aspect, a binding protein (also referred to as “format 6”) is disclosed which contains at least two polypeptide chains, namely, first and second polypeptide chains. In certain embodiments, the first polypeptide chain may contain a structure represented by the formula VL1-(X1)n-VH1-(X2)n-CL-(X3)n, wherein VL1 is a first light chain variable domain, VH1 is a first heavy chain variable domain, CL is a light chain constant domain, X1 and X2 are linkers with the proviso that they are not a constant domain, X3 may comprise an Fc region, and n may be 0 or 1. In certain embodiments, the second polypeptide chain may contain a structure represented by the formula VL2-(X4)n-VH2-(X5)n CH-(X6)n, wherein VL2 is a second light chain variable domain, VH2 is a second heavy chain variable domain, CH is a heavy chain constant domain, X4 and X5 are linkers with the proviso that they are not a constant domain, X6 may comprise an Fc region with the proviso that if X3 contains an Fc region, then X6 does not contain an Fc region, and n may be 0 or 1. In certain embodiments, the VL1 and VH1 domains may form one functional binding site for antigen A, while the VL2 and VH2 domains may form one functional binding site for antigen B. In certain embodiments, the binding protein is capable of binding both antigens A and B simultaneously.

In another aspect, a binding protein (also referred to as “format 7”) is disclosed which contains at least two polypeptide chains, namely, first and second polypeptide chains. In certain embodiments, the first polypeptide chain may contain a structure represented by the formula VL1-(X1)n-VH1-(X2)n-CL-(X3)n, wherein VL1 is a first light chain variable domain, VH1 is a first heavy chain variable domain, CL is a light chain constant domain, X1 and X2 are linkers with the proviso that they are not a constant domain, X3 may comprise an Fc region, and n may be 0 or 1. In certain embodiments, the second polypeptide chain may contain a structure represented by the formula VH2-(X4)n-VL2-(X5)n-CH-(X6)n, wherein VH2 is a second heavy chain variable domain, VL2 is a second light chain variable domain, CH is a heavy chain constant domain, X4 and X5 are linkers with the proviso that they are not a constant domain, X6 may comprise an Fc region with the proviso that if X3 contains an Fc region, then X6 does not contain an Fc region, and n may be 0 or 1. In certain embodiments, the VL1 and VH1 domains may form one functional binding site for antigen A, while the VL2 and VH2 domains may form one functional binding site for antigen B. In certain embodiments, the binding protein is capable of binding both antigens A and B simultaneously.

In another aspect, a binding protein (also referred to as “format 8”) is disclosed which contains at least two polypeptide chains, namely, first and second polypeptide chains. In one aspect, the first polypeptide chain may contain a structure represented by the formula VH1-(X1)n-VL1-(X2)n CL-(X3)n, wherein VL1 is a first light chain variable domain, VH1 is a first heavy chain variable domain, CL is a light chain constant domain, X1 and X2 are linkers with the proviso that they are not a constant domain, X3 may comprise an Fc region, and n may be 0 or 1. In certain embodiments, the second polypeptide chain may contain a structure represented by the formula VH2-(X4)n-VL2-(X5)n-CH-(X6)n, wherein VH2 is a second heavy chain variable domain, VL2 is a second light chain variable domain, CH is a heavy chain constant domain, X4 and X5 are linkers with the proviso that they are not a constant domain, X6 may comprise an Fc region with the proviso that if X3 contains an Fc region, then X6 does not contain an Fc region, and n may be 0 or 1. In certain embodiments, the VL1 and VH1 domains may form one functional binding site for antigen A, while the VL2 and VH2 domains may form one functional binding site for antigen B. In certain embodiments, the binding protein is capable of binding both antigens A and B simultaneously.

In another aspect, a binding protein (also referred to as “format 9”) is disclosed which contains at least two polypeptide chains, namely, first and second polypeptide chains. In one aspect, the first polypeptide chain may contain a structure represented by the formula VH1-(X1)n-VL1-(X2)n-CL-(X3)n, wherein VL1 is a first light chain variable domain, VH1 is a first heavy chain variable domain, CL is a light chain constant domain, X1 and X2 are linkers with the proviso that they are not a constant domain, X3 may comprise an Fc region, and n may be 0 or 1. In certain embodiments, the second polypeptide chain may contain a structure represented by the formula VL2-(X4)n-VH2-(X5)n-CH-(X6)n, wherein VH2 is a second heavy chain variable domain, VL2 is a second light chain variable domain, CH is a heavy chain constant domain, X4 and X5 are linkers with the proviso that they are not a constant domain, X6 may comprise an Fc region with the proviso that if X3 contains an Fc region, then X6 does not contain an Fc region, and n may be 0 or 1. In certain embodiments, the VL1 and VH1 domains may form one functional binding site for antigen A, while the VL2 and VH2 domains may form one functional binding site for antigen B. In certain embodiments, the binding protein is capable of binding both antigens A and B simultaneously.

In another aspect, a binding protein (also referred to as “format 10”) is disclosed which contains at least two polypeptide chains, namely, first and second polypeptide chains. In one aspect, the first polypeptide chain may contain a structure represented by the formula VL1-(X1)n-VH2-(X2)n-CL-(X3)n, wherein VL1 is a first light chain variable domain, VH2 is a second heavy chain variable domain, CL is a light chain constant domain, X1 and X2 are linkers with the proviso that they are not a constant domain, X3 may comprise an Fc region, and n may be 0 or 1. In certain embodiments, the second polypeptide chain may contain a structure represented by the formula VL2-(X4)n-VH1-(X5)n-CH-(X6)n, wherein VH1 is a first heavy chain variable domain, VL2 is a second light chain variable domain, CH is a heavy chain constant domain, X4 and X5 are linkers with the proviso that they are not a constant domain, X6 may comprise an Fc region with the proviso that if X3 contains an Fc region, then X6 does not contain an Fc region, and n may be 0 or 1. In certain embodiments, the VL1 and VH1 domains may form one functional binding site for antigen A, while the VL2 and VH2 domains may form one functional binding site for antigen B. In certain embodiments, the binding protein is capable of binding both antigens A and B simultaneously.

In another aspect, a binding protein (also referred to as “format 11”) is disclosed which contains at least two polypeptide chains, namely, first and second polypeptide chains. In one aspect, the first polypeptide chain may contain a structure represented by the formula VH1-(X1)n-VL2-(X2)n-CL-(X3)n, wherein VH1 is a first heavy chain variable domain, VL2 is a second light chain variable domain, CL is a light chain constant domain, X1 and X2 are linkers with the proviso that they are not a constant domain, X3 may comprise an Fc region, and n may be 0 or 1. In certain embodiments, the second polypeptide chain may contain a structure represented by the formula VH2-(X4)n-VL1-(X5)n-CH-(X6)n, wherein VH2 is a second heavy chain variable domain, VL1 is a first light chain variable domain, CH is a heavy chain constant domain, X4 and X5 are linkers with the proviso that they are not a constant domain, X6 may comprise an Fc region with the proviso that if X3 contains an Fc region, then X6 does not contain an Fc region, and n may be 0 or 1. In certain embodiments, the VL1 and VH1 domains may form one functional binding site for antigen A, while the VL2 and VH2 domains may form one functional binding site for antigen B. In certain embodiments, the binding protein is capable of binding both antigens A and B simultaneously.

In another aspect, a binding protein (also referred to as “format 12”) is disclosed which contains at least two polypeptide chains, namely, first and second polypeptide chains. In one aspect, the first polypeptide chain may contain a structure represented by the formula VH1-(X1)n-VH2-(X2)n-CL-(X3)n, wherein VH1 is a first heavy chain variable domain, VH2 is a second heavy chain variable domain, CL is a light chain constant domain, X1 and X2 are linkers with the proviso that they are not a constant domain, X3 may comprise an Fc region, and n may be 0 or 1. In certain embodiments, the second polypeptide chain may contain a structure represented by the formula VL2-(X4)n-VL1-(X5)n-CH-(X6)n, wherein VL1 is a first light chain variable domain, VL2 is a second light chain variable domain, CH is a heavy chain constant domain, X4 and X5 are linkers with the proviso that they are not a constant domain, X6 may comprise an Fc region with the proviso that if X3 contains an Fc region, then X6 does not contain an Fc region, and n may be 0 or 1. In certain embodiments, the VL1 and VH1 domains may form one functional binding site for antigen A, while the VL2 and VH2 domains may form one functional binding site for antigen B. In certain embodiments, the binding protein is capable of binding both antigens A and B simultaneously.

In certain embodiments, the binding proteins disclosed herein comprise linkers X1, X2, X4, and X5 which are independently selected from the group consisting of SEQ IDs NOs; X, Y, Z etc set forth in Table 5 herein.

TABLE 5
Examples of linkers suiable for use
the binding protins disclosed herein

| Amino acid Sequence | SEQ ID No: |
|---|---|
| G | |
| GG | |
| GGS | |
| GGGS | |
| GGGGS | |
| GGGSG | |
| GGGSGGG | |
| GGGGSGGG | |
| LGGCGGGS | |
| GGGGSGGGGS | |

In certain embodiments, the linkers X1, X2, X4, and X5 consist of amino acid sequences which are independently selected from the group consisting of SEQ IDs NOs; X, Y, Z etc set forth in Table 5 herein.

In certain embodiments, the linkers X1, X2, X4, and X5 consist of amino acid sequences which are independently selected from the amino acid sequences set forth in Table 5 herein. In certain embodiments, the binding proteins disclosed herein comprise a combination of linkers X1, X2, X4 and X5 set forth in Table 6 herein.

TABLE 6
Examples of linker combinations suiable for use in
the binding protins disclosed herein

| | X4 amino | X5 amino | X1 amino | X2 amino |
|---|---|---|---|---|
| | acid | acid | acid | acid |
| For- | sequence | sequence | sequence | sequence |
| mat | (SEQ ID NO) | (SEQ ID NO) | (SEQ ID NO) | (SEQ ID NO) |
| 2 | G | GG | GGGSGGG | GGGSG |
| 10 | GGGSGGGG | LGGCGGGS | GGGSGGGG | LGGCGGGS |
| 11 | GGGGSGGG | GGGSG | GGGGSGGG | GGG |
| 11 | GGGGSGGG | GGG | GGGGSGGG | GGSGG |
| 12 | GGGGGGG | GGGGG | G | GG |

In certain embodiments, the binding proteins disclosed herein comprise a first polypeptide chain comprising the amino acid sequences set forth in Tables 21-24 herein. In certain embodiments, the binding proteins disclosed herein comprise a second polypeptide chain comprising the amino acid sequences set forth in Tables 21-24 herein. In certain embodiments, the binding proteins disclosed herein comprise a complementarry pair of first and second polypeptide chains comprising the amino acid sequences set forth in Tables 21-24 herein.

In certain embodiments, the binding proteins disclosed herein are full-length coDVD-Ig molecules.

In certain embodiments, the binding proteins disclosed herein further comprising a cell surface anchoring moiety linked to the N and/or C terminus of the first or polypeptide chain. In certain embodiments, the anchoring moiety comprises the Aga2p polypeptide.

In one aspect, in order to form a functional DVD-Ig molecule in formats 3, 4, and 5, VD1s from different polypeptide chains form N-terminal (outer) antigen binding domain, while VD2s from different polypeptide chains form C-terminal (inner) antigen binding domain. These two antigen binding domains are in series orientation. In format 1 and format 5, VD1 and VD2 in the same polypeptide chain have the same chain type and will not pair with each other. In formats 3 and 4, VD1 and VD2 in same polypeptide chain have different chain type. VD1 and VD2 from the same polypeptide chain may potentially pair with each other, which is not preferred. In one aspect, the linker between VD1 and VD2 is shorter than 12 amino acids in order to limit such potential pairing between VD1 and VD2. In certain embodiments, the DVD-Ig molecules in format 3, 4, and 5 also face the same challenges as format 1 DVD-Ig in that N-terminal (outer) antigen binding domain may potentially block C-terminal (inner) antigen binding domain.

In formats 6, 7, 8 and 9, VD1 and VD2 in one polypeptide chain have different chain type. To form functional DVD-Ig molecule in formats 6, 7, 8, and 9, VD1 and VD2 in the same polypeptide chain form antigen binding domain in scFv format. In the case of formats 6, 7, 8 and 9, it is preferred that the length of the linker between VD1 and VD2 be longer than 12 amino acids to provide sufficient spatial flexibility for VD1 and VD2 to form a natural Fv. In other words, DVD-Ig molecules in formats 6, 7, 8 and 9 are formed by scFv-CL and scFv-CH1-Fc. The stability of the scFv format may be a concern for these formats.

Similar to format 2, to form a functional DVD-Ig molecule in formats 10, 11 and 12, VD1 from one polypeptide chain and VD2 from another polypeptide chain form two antigen binding domains. These two antigen binding domains are in cross-over (side-by-side) orientation, which provide the potential to fully expose both antigen binding domains. In formats 2 and 12, VD1 and VD2 in same polypeptide chain have same chain type and will not pair with each other. In formats 10 and 11, however, VD1 and VD2 in same polypeptide chain have different chain type. Therefore, VD1 and VD2 from same polypeptide chain in formats 10 and 11 may potentially pair with each other, which is not preferred. To prevent such undesirable pairing in formats 10 and 11, the linker length between VD1 and VD2 in the same polypeptide chain is preferably shorter than 12 amino acids to form bispecific diabody by two different polypeptide chains.

A molecule similar to format 10 is described in U.S. Pat. Application US 20090060910A1, which describes a dual affinity retargeting reagent (DART)-Ig molecule comprising four polypeptide chains: two polypeptide chains with the configuration of VL1-linker-VH2-linker-CL; and two polypeptide chains with configuration of VL2-linker-VH1-linker-CH1-Fc. DART-Ig molecule's expression and physical chemical properties may be improved by optimizing the linker between variable domain and constant domain.

In one embodiment, antigens A and B are different antigens. In another embodiment, antigens A and B are the same antigen.

In one embodiment, the VL1 and VL2 may be any one of the light chain variable domains disclosed in U.S. Pat. No. 7,612,181 and U.S. Pat. No. 8,258,268. In another embodiment, the VH1 and VH2 may be any one of the heavy chain variable domains disclosed in U.S. Pat. No. 7,612,181 and U.S. Pat. No. 8,258,268.

Method for generating the expressing constructs for the cross-over DVD-Ig, methods for transfecting the constructs into host cells and purifying and characterizing the expressed proteins are as those disclosed in U.S. Pat. No. 7,612,181 and U.S. Pat. No. 8,258,268, which are expressly incorporated into the present disclosure by reference.

In another embodiment, the binding protein has an on rate constant (Kₒₙ) to one or more targets of at least about 10² M⁻¹s⁻¹; at least about 10³ M⁻¹s⁻¹; at least about 10⁴ M⁻¹s⁻¹; at least about 10⁵ M⁻¹s⁻¹; or at least about 10⁶ M⁻¹s⁻¹, as measured by surface plasmon resonance. In an embodiment, the binding protein has an on rate constant (Kₒₙ) to one or more targets from about 10² M⁻¹s⁻¹ to about 10³ M⁻¹s⁻¹; from about 10³ M⁻¹s⁻¹ to about 10⁴ M⁻¹s⁻¹; from about 10⁴ M⁻¹s⁻¹ to about 10⁵ M⁻¹s⁻¹; or from about 10⁵ M⁻¹s⁻¹ to about 10⁶ M⁻¹s⁻¹, as measured by surface plasmon resonance.

In another embodiment, the binding protein has an off rate constant (K_{off}) for one or more targets of at most about 10³ s⁻¹; at most about 10⁴ s⁻¹; at most about 10⁻⁵ s⁻¹; or at most about 10⁻⁶ s⁻¹, as measured by surface plasmon resonance. In an embodiment, the binding protein has an off rate constant (K_{off}) to one or more targets of about 10⁻³ s⁻¹ to about 10⁻⁴ s⁻¹; of about 10⁻⁴ s⁻¹ to about 10⁻⁵ s⁻¹; or of about 10⁻⁵ s⁻¹ to about 10⁻⁶ s⁻¹, as measured by surface plasmon resonance.

In another embodiment, the binding protein has a dissociation constant (K_{d}) to one or more targets of at most about 10⁻⁷M; at most about 10⁻⁸M; at most about 10⁻⁹M; at most about 10⁻¹⁹M; at most about 10⁻¹¹M; at most about 10⁻¹²M; or at most 10⁻¹³M. In an embodiment, the binding protein has a dissociation constant (K_{d}) to its targets of about 10⁻⁷M to about 10⁻⁸M; of about 10⁻⁸M to about 10⁻⁹M; of about 10⁻⁹M to about 10⁻¹⁹M; of about 10⁻¹⁹M to about 10⁻¹¹M; of about 10⁻¹¹M to about 10⁻¹²M; or of about 10⁻¹² to M about 10⁻¹³M.

In another embodiment, the binding protein is a conjugate further comprising an agent. In an embodiment, the agent is an immunoadhesion molecule, an imaging agent, a therapeutic agent, or a cytotoxic agent. In an embodiment, the imaging agent is a radiolabel, an enzyme, a fluorescent label, a luminescent label, a bioluminescent label, a magnetic label, or biotin. In another embodiment, the radiolabel is ³H, ¹⁴C, ³⁵S, ⁹⁹Y, ⁹⁹Tc, ¹¹¹In, ¹²⁵I, ¹³¹I, ¹⁷⁷Lu, ¹⁶⁶Ho, or ¹⁵³Sm. In yet another embodiment, the therapeutic or cytotoxic agent is an anti-metabolite, an alkylating agent, an antibiotic, a growth factor, a cytokine, an anti-angiogenic agent, an anti-mitotic agent, an anthracycline, toxin, or an apoptotic agent.

In another embodiment, the binding protein is a crystallized binding protein and exists as a crystal. In an embodiment, the crystal is a carrier-free pharmaceutical controlled release crystal. In another embodiment, the crystallized binding protein has a greater half life in vivo than the soluble counterpart of the binding protein. In yet another embodiment, the crystallized binding protein retains biological activity.

In another embodiment, the binding protein described herein is glycosylated. For example, the glycosylation pattern is a human glycosylation pattern.

An isolated nucleic acid encoding any one of the binding proteins disclosed herein is also provided. A further embodiment provides a vector comprising the isolated nucleic acid disclosed herein wherein the vector is pcDNA; pTT (Durocher et al. (2002) Nucleic Acids Res. 30(2); pTT3 (pTT with additional multiple cloning site); pEFBOS, see Mizushima and Nagata (1990) Nucleic Acids Res. 18(17); pBV; pJV; pcDNA3.1 TOPO; pEF6 TOPO; pBOS; pHybE; or pBJ. In an embodiment, the vector is a vector disclosed in US Patent Publication No. 20090239259.

In another aspect, a host cell is transformed with the vector disclosed herein. In an embodiment, the host cell is a prokaryotic cell, for example, E. coli. In another embodiment, the host cell is a eukaryotic cell, for example, a protist cell, an animal cell, a plant cell, or a fungal cell. In an embodiment, the host cell is a mammalian cell including, but not limited to, 293E, CHO, COS, NS0, SP2, PER.C6, or a fungal cell, such as Saccharomyces cerevisiae, or an insect cell, such as Sf9. In an embodiment, two or more binding proteins, e.g., with different specificities, are produced in a single recombinant host cell. For example, the expression of a mixture of antibodies has been called Oligoclonics™ (Merus B.V., The Netherlands), see U.S. Pat. Nos. 7,262,028 and 7,429,486.

A method of producing a binding protein disclosed herein comprising culturing any one of the host cells disclosed herein in a culture medium under conditions sufficient to produce the binding protein is provided.

One embodiment provides a composition for the release of a binding protein wherein the composition comprises a crystallized binding protein, an ingredient, and at least one polymeric carrier. In an embodiment, the polymeric carrier is poly (acrylic acid), a poly (cyanoacrylate), a poly (amino acid), a poly (anhydride), a poly (depsipeptide), a poly (ester), poly (lactic acid), poly (lactic-co-glycolic acid) or PLGA, poly (b-hydroxybutryate), poly (caprolactone), poly (dioxanone), poly (ethylene glycol), poly ((hydroxypropyl) methacrylamide, poly [(organo)phosphazene], a poly (ortho ester), poly (vinyl alcohol), poly (vinylpyrrolidone), a maleic anhydride-alkyl vinyl ether copolymer, a pluronic polyol, albumin, alginate, cellulose, a cellulose derivative, collagen, fibrin, gelatin, hyaluronic acid, an oligosaccharide, a glycaminoglycan, a sulfated polysaccharide, or blends and copolymers thereof. In an embodiment, the ingredient is albumin, sucrose, trehalose, lactitol, gelatin, hydroxypropyl-β-cyclodextrin, methoxypolyethylene glycol, or polyethylene glycol.

Another embodiment provides a method for treating a mammal comprising the step of administering to the mammal an effective amount of a composition disclosed herein.

A pharmaceutical composition comprising a binding protein disclosed herein and a pharmaceutically acceptable carrier is provided. In a further embodiment, the pharmaceutical composition comprises at least one additional therapeutic agent for treating a disorder. For example, the additional agent may be a therapeutic agent, an imaging agent, a cytotoxic agent, an angiogenesis inhibitor (including but not limited to an anti-VEGF antibody or a VEGF-trap), a kinase inhibitor (including but not limited to a KDR and a TIE-2 inhibitor), a co-stimulation molecule blocker (including but not limited to anti-B7.1, anti-B7.2, CTLA4-Ig, anti-CD20), an adhesion molecule blocker (including but not limited to an anti-LFA-1 antibody, an anti-E/L selectin antibody, a small molecule inhibitor), an anti-cytokine antibody or functional fragment thereof (including but not limited to an anti-IL-18, an anti-TNF, and an anti-IL-6/cytokine receptor antibody), methotrexate, cyclosporin, rapamycin, FK506, a detectable label or reporter, a TNF antagonist, an antirheumatic, a muscle relaxant, a narcotic, a non-steroid anti-inflammatory drug (NSAID), an analgesic, an anesthetic, a sedative, a local anesthetic, a neuromuscular blocker, an antimicrobial, an antipsoriatic, a corticosteriod, an anabolic steroid, an erythropoietin, an immunization, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, or a cytokine antagonist.

A method for treating a human subject suffering from a disorder in which the target, or targets, capable of being bound by the binding protein disclosed herein is detrimental, comprising administering to the human subject a binding protein disclosed herein such that the activity of the target, or targets, in the human subject is inhibited and one or more symptoms is alleviated or treatment is achieved is provided. The binding proteins provided herein can be used to treat humans suffering from autoimmune diseases such as, for example, those associated with inflammation. In an embodiment, the binding proteins provided herein or antigen-binding portions thereof, are used to treat asthma, allergies, allergic lung disease, allergic rhinitis, atopic dermatitis, chronic obstructive pulmonary disease (COPD), fibrosis, cystic fibrosis (CF), fibrotic lung disease, idiopathic pulmonary fibrosis, liver fibrosis, lupus, hepatitis B-related liver diseases and fibrosis, sepsis, systemic lupus erythematosus (SLE), glomerulonephritis, inflammatory skin diseases, psoriasis, diabetes, insulin dependent diabetes mellitus, infectious diseases caused by HIV, inflammatory bowel disease (IBD), ulcerative colitis (UC), Crohn's disease (CD), rheumatoid arthritis (RA), osteoarthritis (OA), multiple sclerosis (MS), graft-versus-host disease (GVHD), transplant rejection, ischemic heart disease (IHD), celiac disease, contact hypersensitivity, alcoholic liver disease, Behcet's disease, atherosclerotic vascular disease, occular surface inflammatory diseases, or Lyme disease.

In another embodiment, the disorder or condition to be treated comprises the symptoms caused by viral infection in a human which is caused by, for example, HIV, the human rhinovirus, an enterovirus, a coronavirus, a herpes virus, an influenza virus, a parainfluenza virus, a respiratory syncytial virus or an adenovirus.

The binding proteins provided herein can be used to treat neurological disorders. In an embodiment, the binding proteins provided herein, or antigen-binding portions thereof, are used to treat neurodegenerative diseases and conditions involving neuronal regeneration and spinal cord injury.

In an embodiment, diseases that can be treated or diagnosed with the compositions and methods disclosed herein include, but are not limited to, primary and metastatic cancers, including carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract (including cervix, uterus, and ovaries as well as choriocarcinoma and gestational trophoblastic disease), male genital tract (including prostate, seminal vesicles, testes and germ cell tumors), endocrine glands (including the thyroid, adrenal, and pituitary glands), and skin, as well as hemangiomas, melanomas, sarcomas (including those arising from bone and soft tissues as well as Kaposi's sarcoma), tumors of the brain, nerves, eyes, and meninges (including astrocytomas, gliomas, glioblastomas, retinoblastomas, neuromas, neuroblastomas, Schwannomas, and meningiomas), solid tumors arising from hematopoietic malignancies such as leukemias, and lymphomas (both Hodgkin's and non-Hodgkin's lymphomas).

Another embodiment provides for the use of the binding protein in the treatment of a disease or disorder, wherein said disease or disorder is rheumatoid arthritis, osteoarthritis, juvenile chronic arthritis, septic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, Addison's disease, sporadic, polyglandular deficiency type I and polyglandular deficiency type II, Schmidt's syndrome, adult (acute) respiratory distress syndrome, alopecia, alopecia greata, arthropathy, Reiter's disease, psoriatic arthropathy, ulcerative colitic arthropathy, enteropathic synovitis, chlamydia, yersinia and salmonella associated arthropathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, acquired immunodeficiency related diseases, hepatitis B, hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, fibrosis, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Stiffs disease, systemic sclerosis, Sjörgren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo acute liver disease, chronic liver diseases, alcoholic cirrhosis, alcohol-induced liver injury, choleosatatis, idiosyncratic liver disease, drug-induced hepatitis, non-alcoholic steatohepatitis, allergy and asthma, group B streptococci (GBS) infection, mental disorders, depression, schizophrenia, Th2 Type and Th1 Type mediated diseases, acute and chronic pain, different forms of pain, cancers, lung cancer, breast cancer, stomach cancer, bladder cancer, colon cancer, pancreatic cancer, ovarian cancer, prostate cancer, rectal cancer, hematopoietic malignancies, leukemia, lymphoma, Abetalipoprotemia, acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, aerial ectopic beats, AIDS dementia complex, alcohol-induced hepatitis, allergic conjunctivitis, allergic contact dermatitis, allergic rhinitis, allograft rejection, alpha-1-antitrypsin deficiency, amyotrophic lateral sclerosis, anemia, angina pectoris, anterior horn cell degeneration, anti cd3 therapy, antiphospholipid syndrome, anti-receptor hypersensitivity reactions, aortic and peripheral aneuryisms, aortic dissection, arterial hypertension, arteriosclerosis, arteriovenous fistula, ataxia, atrial fibrillation (sustained or paroxysmal), atrial flutter, atrioventricular block, B cell lymphoma, bone graft rejection, bone marrow transplant (BMT) rejection, bundle branch block, Burkitt's lymphoma, burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chronic myelocytic leukemia (CML), chronic alcoholism, chronic inflammatory pathologies, chronic lymphocytic leukemia (CLL), chronic obstructive pulmonary disease (COPD), chronic salicylate intoxication, colorectal carcinoma, congestive heart failure, conjunctivitis, contact dermatitis, cor pulmonale, coronary artery disease, Creutzfeldt-Jakob disease, culture negative sepsis, cystic fibrosis, cytokine therapy associated disorders, dementia pugilistica, demyelinating diseases, dengue hemorrhagic fever, dermatitis, dermatologic conditions, diabetes, diabetes mellitus, diabetic ateriosclerotic disease, diffuse Lewy body disease, dilated congestive cardiomyopathy, disorders of the basal ganglia, Down's syndrome in middle age, drug-induced movement disorders induced by drugs which block CNS dopamine receptors, drug sensitivity, eczema, encephalomyelitis, endocarditis, endocrinopathy, epiglottitis, epstein-barr virus infection, erythromelalgia, extrapyramidal and cerebellar disorders, familial hematophagocytic lymphohistiocytosis, fetal thymus implant rejection, Friedreich's ataxia, functional peripheral arterial disorders, fungal sepsis, gas gangrene, gastric ulcer, glomerular nephritis, graft rejection of any organ or tissue, gram negative sepsis, gram positive sepsis, granulomas due to intracellular organisms, hairy cell leukemia, Hallervorden-Spatz disease, Hashimoto's thyroiditis, hay fever, heart transplant rejection, hemachromatosis, hemodialysis, hemolytic uremic syndrome/thrombolytic thrombocytopenic purpura, hemorrhage, hepatitis A, His bundle arrythmias, HIV infection/HIV neuropathy, Hodgkin's disease, hyperkinetic movement disorders, hypersensitity reactions, hypersensitivity pneumonitis, hypertension, hypokinetic movement disorders, hypothalamic-pituitary-adrenal axis evaluation, idiopathic Addison's disease, idiopathic pulmonary fibrosis, antibody mediated cytotoxicity, Asthenia, infantile spinal muscular atrophy, inflammation of the aorta, influenza a, ionizing radiation exposure, iridocyclitis/uveitis/optic neuritis, ischemia-reperfusion injury, ischemic stroke, juvenile rheumatoid arthritis, juvenile spinal muscular atrophy, Kaposi's sarcoma, kidney transplant rejection, legionella, leishmaniasis, leprosy, lesions of the corticospinal system, lipedema, liver transplant rejection, lymphederma, malaria, malignamt lymphoma, malignant histiocytosis, malignant melanoma, meningitis, meningococcemia, metabolic/idiopathic, migraine headache, mitochondrial multi.system disorder, mixed connective tissue disease, monoclonal gammopathy, multiple myeloma, multiple systems degenerations (Mencel Dejerine-Thomas Shi-Drager and Machado-Joseph), mycobacterium avium intracellulare, mycobacterium tuberculosis, myelodyplastic syndrome, myocardial infarction, myocardial ischemic disorders, nasopharyngeal carcinoma, neonatal chronic lung disease, nephritis, nephrosis, neurodegenerative diseases, neurogenic muscular atrophies, neutropenic fever, non-hodgkins lymphoma, occlusion of the abdominal aorta and its branches, occulsive arterial disorders, okt3 therapy, orchitis/epidydimitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, peripheral atherlosclerotic disease, peripheral vascular disorders, peritonitis, pernicious anemia, pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, preeclampsia, progressive supranucleo palsy, primary pulmonary hypertension, radiation therapy, Raynaud's phenomenon and disease, Raynoud's disease, Refsum's disease, regular narrow QRS tachycardia, renovascular hypertension, reperfusion injury, restrictive cardiomyopathy, sarcomas, scleroderma, senile chorea, senile dementia of Lewy body type, seronegative arthropathies, shock, sickle cell anemia, skin allograft rejection, skin changes syndrome, small bowel transplant rejection, solid tumors, specific arrythmias, spinal ataxia, spinocerebellar degenerations, streptococcal myositis, structural lesions of the cerebellum, subacute sclerosing panencephalitis, syncope, syphilis of the cardiovascular system, systemic anaphalaxis, systemic inflammatory response syndrome, systemic onset juvenile rheumatoid arthritis, T-cell or FAB ALL telangiectasia, thromboangitis obliterans, thrombocytopenia, toxicity, transplants, trauma/hemorrhage, type III hypersensitivity reactions, type IV hypersensitivity, unstable angina, uremia, urosepsis, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, xenograft rejection of any organ or tissue, acute coronary syndromes, acute idiopathic polyneuritis, acute inflammatory demyelinating polyradiculoneuropathy, acute ischemia, adult Still's disease, anaphylaxis, anti-phospholipid antibody syndrome, aplastic anemia, atopic eczema, atopic dermatitis, autoimmune dermatitis, autoimmune disorder associated with streptococcus infection, autoimmune enteropathy, autoimmune hearing loss, autoimmune lymphoproliferative syndrome (ALPS), autoimmune myocarditis, autoimmune premature ovarian failure, blepharitis, bronchiectasis, bullous pemphigoid, cardiovascular disease, catastrophic antiphospholipid syndrome, celiac disease, cervical spondylosis, chronic ischemia, cicatricial pemphigoid, clinically isolated syndrome (cis) with risk for multiple sclerosis, childhood onset psychiatric disorder, dacryocystitis, dermatomyositis, diabetic retinopathy, disk herniation, disk prolaps, drug induced immune hemolytic anemia, endometriosis, endophthalmitis, episcleritis, erythema multiforme, erythema multiforme major, gestational pemphigoid, Guillain-Barré syndrome (GBS), Hughes syndrome, idiopathic Parkinson's disease, idiopathic interstitial pneumonia, IgE-mediated allergy, immune hemolytic anemia, inclusion body myositis, infectious ocular inflammatory disease, inflammatory demyelinating disease, inflammatory heart disease, inflammatory kidney disease, IPF/UIP, iritis, keratitis, keratojuntivitis sicca, Kussmaul disease or Kussmaul-Meier disease, Landry's paralysis, Langerhan's cell histiocytosis, livedo reticularis, macular degeneration, microscopic polyangiitis, morbus bechterev, motor neuron disorders, mucous membrane pemphigoid, multiple organ failure, myasthenia gravis, myelodysplastic syndrome, myocarditis, nerve root disorders, neuropathy, non-A non-B hepatitis, optic neuritis, osteolysis, pauciarticular JRA, peripheral artery occlusive disease (PAOD), peripheral vascular disease (PVD), peripheral artery, disease (PAD), phlebitis, polyarteritis nodosa (or periarteritis nodosa), polychondritis, poliosis, polyarticular JRA, polyendocrine deficiency syndrome, polymyositis, polymyalgia rheumatica (PMR), primary Parkinsonism, prostatitis, pure red cell aplasia, primary adrenal insufficiency, recurrent neuromyelitis optica, restenosis, rheumatic heart disease, sapho (synovitis, acne, pustulosis, hyperostosis, and osteitis), secondary amyloidosis, shock lung, scleritis, sciatica, secondary adrenal insufficiency, silicone associated connective tissue disease, sneddon-wilkinson dermatosis, spondilitis ankylosans, Stevens-Johnson syndrome (SJS), temporal arteritis, toxoplasmic retinitis, toxic epidermal necrolysis, transverse myelitis, TRAPS (tumor necrosis factor receptor, type 1 allergic reaction, type II diabetes, urticaria, usual interstitial pneumonia (UIP), vasculitis, vernal conjunctivitis, viral retinitis, Vogt-Koyanagi-Harada syndrome (VKH syndrome), wet macular degeneration, or wound healing.

In an embodiment, the binding proteins, or antigen-binding portions thereof, are used to treat cancer or in the prevention or inhibition of metastases from the tumors described herein either when used alone or in combination with radiotherapy and/or chemotherapeutic agents.

In another aspect, methods of treating a patient suffering from a disorder comprising the step of administering any one of the binding proteins disclosed herein before, concurrently, or after the administration of a second agent, are provided. In an embodiment, the second agent is budenoside, epidermal growth factor, a corticosteroid, cyclosporin, sulfasalazine, an aminosalicylate, 6-mercaptopurine, azathioprine, metronidazole, a lipoxygenase inhibitor, mesalamine, olsalazine, balsalazide, an antioxidant, a thromboxane inhibitor, an IL-1 receptor antagonist, an anti-IL-1β mAbs, an anti-IL-6 or IL-6 receptor mAb, a growth factor, an elastase inhibitor, a pyridinyl-imidazole compound, an antibody or agonist of TNF, LT, IL-1, IL-2, IL-6, IL-7, IL-8, IL-12, IL-13, IL-15, IL-16, IL-18, IL-23, EMAP-II, GM-CSF, FGF, or PDGF, an antibody to CD2, CD3, CD4, CD8, CD-19, CD25, CD28, CD30, CD40, CD45, CD69, CD90 or a ligand thereof, methotrexate, cyclosporin, FK506, rapamycin, mycophenolate mofetil, leflunomide, an NSAID, ibuprofen, prednisolone, a phosphodiesterase inhibitor, an adenosine agonist, an antithrombotic agent, a complement inhibitor, an adrenergic agent, IRAK, NIK, IKK, p38, a MAP kinase inhibitor, an IL-1β converting enzyme inhibitor, a TNFα-converting enzyme inhibitor, a T-cell signalling inhibitor, a metalloproteinase inhibitor, sulfasalazine, azathioprine, a 6-mercaptopurine, an angiotensin converting enzyme inhibitor, a soluble cytokine receptor, a soluble p55 TNF receptor, a soluble p75 TNF receptor, sIL-1RI, sIL-1RII, sIL-6R, an antiinflammatory cytokine, IL-4, IL-10, IL-11, IL-13, or TGFβ. In a particular embodiment, the pharmaceutical compositions disclosed herein are administered to a patient by parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal administration.

Anti-idiotype antibodies to the binding proteins disclosed herein are also provided. An anti-idiotype antibody includes any protein or peptide-containing molecule that comprises at least a portion of an immunoglobulin molecule such as, but not limited to, at least one complementarily determining region (CDR) of a heavy or light chain or a ligand binding portion thereof, a heavy chain or light chain variable region, a heavy chain or light chain constant region, a framework region, or any portion thereof, that can be incorporated into a binding protein provided herein.

A method of determining the presence, amount or concentration of the target antigen, or fragment thereof, in a test sample is provided. The method comprises assaying the test sample for the antigen, or fragment thereof, by an immunoassay. The immunoassay (i) employs at least one binding protein and at least one detectable label and (ii) comprises comparing a signal generated by the detectable label as a direct or indirect indication of the presence, amount or concentration of the antigen, or fragment thereof, in the test sample to a signal generated as a direct or indirect indication of the presence, amount or concentration of the antigen, or fragment thereof, in a control or a calibrator. The calibrator is optionally part of a series of calibrators in which each of the calibrators differs from the other calibrators in the series by the concentration of the antigen, or fragment thereof. The method may comprise (i) contacting the test sample with at least one capture agent, which binds to an epitope on the antigen, or fragment thereof, so as to form a capture agent/antigen, or fragment thereof, complex, (ii) contacting the capture agent/antigen, or fragment thereof, complex with at least one detection agent, which comprises a detectable label and binds to an epitope on the antigen, or fragment thereof, that is not bound by the capture agent, to form a capture agent/antigen, or fragment thereof/detection agent complex, and (iii) determining the presence, amount or concentration of the antigen, or fragment thereof, in the test sample based on the signal generated by the detectable label in the capture agent/antigen, or fragment thereof/detection agent complex formed in (ii), wherein at least one capture agent and/or at least one detection agent is the at least one binding protein.

Alternatively, the method may include (i) contacting the test sample with at least one capture agent, which binds to an epitope on the antigen, or fragment thereof, so as to form a capture agent/antigen, or fragment thereof, complex, and simultaneously or sequentially, in either order, contacting the test sample with detectably labeled antigen, or fragment thereof, which can compete with any antigen, or fragment thereof, in the test sample for binding to the at least one capture agent, wherein any antigen, or fragment thereof, present in the test sample and the detectably labeled antigen compete with each other to form a capture agent/antigen, or fragment thereof, complex and a capture agent/detectably labeled antigen, or fragment thereof, complex, respectively, and (ii) determining the presence, amount or concentration of the antigen, or fragment thereof, in the test sample based on the signal generated by the detectable label in the capture agent/detectably labeled antigen, or fragment thereof, complex formed in (ii), wherein at least one capture agent is the at least one binding protein and wherein the signal generated by the detectable label in the capture agent/detectably labeled antigen, or fragment thereof, complex is inversely proportional to the amount or concentration of antigen, or fragment thereof, in the test sample.

The test sample may be from a patient, in which case the method may further include diagnosing, prognosticating, or assessing the efficacy of therapeutic/prophylactic treatment of the patient. If the method include assessing the efficacy of therapeutic/prophylactic treatment of the patient, the method optionally further comprises modifying the therapeutic/prophylactic treatment of the patient as needed to improve efficacy. The method may be adapted for use in an automated system or a semi-automated system. Accordingly, the methods described herein also can be used to determine whether or not a subject has or is at risk of developing a given disease, disorder or condition. Specifically, such a method may include the steps of: (a) determining the concentration or amount in a test sample from a subject of analyte, or fragment thereof, (e.g., using the methods described herein, or methods known in the art); and (b) comparing the concentration or amount of analyte, or fragment thereof, determined in step (a) with a predetermined level, wherein, if the concentration or amount of analyte determined in step (a) is favorable with respect to a predetermined level, then the subject is determined not to have or be at risk for a given disease, disorder or condition. However, if the concentration or amount of analyte determined in step (a) is unfavorable with respect to the predetermined level, then the subject is determined to have or be at risk for a given disease, disorder or condition.

Additionally, provided herein is method of monitoring the progression of disease in a subject. Optimally the method may include the steps of: (a) determining the concentration or amount in a test sample from a subject of analyte; (b) determining the concentration or amount in a later test sample from the subject of analyte; and (c) comparing the concentration or amount of analyte as determined in step (b) with the concentration or amount of analyte determined in step (a), wherein if the concentration or amount determined in step (b) is unchanged or is unfavorable when compared to the concentration or amount of analyte determined in step (a), then the disease in the subject is determined to have continued, progressed or worsened. By comparison, if the concentration or amount of analyte as determined in step (b) is favorable when compared to the concentration or amount of analyte as determined in step (a), then the disease in the subject is determined to have discontinued, regressed or improved.

Optionally, the method further comprises comparing the concentration or amount of analyte as determined in step (b), for example, with a predetermined level. Further, optionally the method comprises treating the subject with one or more pharmaceutical compositions for a period of time if the comparison shows that the concentration or amount of analyte as determined in step (b), for example, is unfavorably altered with respect to the predetermined level.

### BRIEF DESCRIPTION OF THE DRAWINGS

FIG. 1 depicts schematic representations of four binding protein formats disclosed herein.

### DETAILED DESCRIPTION

Multi-specific binding proteins are disclosed. In one embodiment, cross-over dual-variable-domain (DVD) Igs are disclosed which are generated by crossing over light chain and the heavy chain variable domains of a dual-variable-domain (DVD) Ig or Ig like protein. In another embodiment, the binding proteins may be modified antibodies that bind to two or more target proteins. The binding to each of the target proteins may be mediated by one, two, three, four, five or more binding domains present on the disclosed multi-specific binding proteins. The binding proteins and pharmaceutical compositions thereof, as well as nucleic acids, recombinant expression vectors and host cells for making such binding proteins are also provided. Methods of using the disclosed binding proteins to detect specific antigens and/or ligands, either in vitro or in vivo, as well as uses in the prevention, and/or treatment diseases and disorders are also provided.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including”, as well as other forms, such as “includes” and “included”, is not limiting.

Generally, nomenclatures used in connection with cell and tissue culture, molecular biology, immunology, microbiology, genetics and protein and nucleic acid chemistry and hybridization described herein are those well known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

That the disclosure may be more readily understood, select terms are defined below.

The term “ligand”, as it is well known and commonly used in the art, refers to any substance capable of binding, or of being bound, to another substance. Similarly, the term “antigen”, as it is well known and commonly used in the art, refers to any substance to which an antibody may be generated. Although “antigen” is commonly used in reference to an antibody binding substrate, and “ligand” is often used when referring to receptor binding substrates, these terms are not distinguishing, one from the other, and encompass a wide range of overlapping chemical entities. For the avoidance of doubt, antigen and ligand are used interchangeably throughout herein. Antigens/ligands may be a peptide, a polypeptide, a protein, an aptamer, a polysaccharide, a sugar molecule, a carbohydrate, a lipid, an oligonucleotide, a polynucleotide, a synthetic molecule, an inorganic molecule, an organic molecule, and any combination thereof.

The term “antibody” refers to an immunoglobulin (Ig) molecule, which is generally comprised of four polypeptide chains, two heavy (H) chains and two light (L) chains, or a functional fragment, mutant, variant, or derivative thereof, that retains the epitope binding features of an Ig molecule. Such fragment, mutant, variant, or derivative antibody formats are known in the art. In an embodiment of a full-length antibody, each heavy chain is comprised of a heavy chain variable region (VH) and a heavy chain constant region (CH). The heavy chain variable region (domain) is also designated as VDH in this disclosure. The CH is comprised of three domains, CH1, CH2 and CH3. Each light chain is comprised of a light chain variable region (VL) and a light chain constant region (CL). The CL is comprised of a single CL domain. The light chain variable region (domain) is also designated as VDL in this disclosure. The VH and VL can be further subdivided into regions of hypervariability, termed complementarity determining regions (CDRs), interspersed with regions that are more conserved, termed framework regions (FRs). Generally, each VH and VL is composed of three CDRs and four FRs, arranged from amino-terminus to carboxy-terminus in the following order: FR1, CDR1, FR2, CDR2, FR3, CDR3, and FR4. Immunoglobulin molecules can be of any type (e.g., IgG, IgE, IgM, IgD, IgA and IgY), class (e.g., IgG1, IgG2, IgG3, IgG4, IgA1 and IgA2), or subclass.

An “affinity matured” antibody is an antibody with one or more alterations in one or more CDRs thereof which result an improvement in the affinity of the antibody for antigen, compared to a parent antibody which does not possess those alteration(s). Exemplary affinity matured antibodies will have nanomolar or even picomolar affinities for the target antigen. Affinity matured antibodies are produced by procedures known in the art. Marks et al. (1992) BioTechnology 10:779-783 describes affinity maturation by VH and VL domain shuffling. Random mutagenesis of CDR and/or framework residues is described by Barbas et al. (1994) Proc. Nat. Acad. Sci. USA 91:3809-3813; Schier et al. (1995) Gene 169:147-155; Yelton et al. (1995) J. Immunol. 155:1994-2004; Jackson et al. (1995) J. Immunol. 154(7):3310-9; Hawkins et al. (1992) J. Mol. Biol. 226:889-896 and mutation at selective mutagenesis positions, contact or hypermutation positions with an activity enhancing amino acid residue as described in U.S. Pat. No. 6,914,128.

The term “CDR-grafted antibody” refers to an antibody that comprises heavy and light chain variable region sequences in which the sequences of one or more of the CDR regions of VH and/or VL are replaced with CDR sequences of another antibody. For example, the two antibodies can be from different species, such as antibodies having murine heavy and light chain variable regions in which one or more of the murine CDRs has been replaced with human CDR sequences.

The term “humanized antibody” refers to an antibody from a non-human species that has been altered to be more “human-like”, i.e., more similar to human germline sequences. One type of humanized antibody is a CDR-grafted antibody, in which non-human CDR sequences are introduced into human VH and VL sequences to replace the corresponding human CDR sequences. A “humanized antibody” is also an antibody or a variant, derivative, analog or fragment thereof that comprises framework region (FR) sequences having substantially (e.g., at least 80%, at least 85%, at least 90%, at least 95%, at least 98% or at least 99% identity to) the amino acid sequence of a human antibody and at least one CDR having substantially the amino acid sequence of a non-human antibody. A humanized antibody may comprise substantially all of at least one, and typically two, variable domains (Fab, Fab′, F(ab′)2, FabC, Fv) in which the sequence of all or substantially all of the CDR regions correspond to those of a non-human immunoglobulin (i.e., donor antibody) and the sequence of all or substantially all of the FR regions are those of a human immunoglobulin. The humanized antibody also may include the CH1, hinge, CH2, CH3, and CH4 regions of the heavy chain. In an embodiment, a humanized antibody also comprises at least a portion of a human immunoglobulin Fc region. In some embodiments, a humanized antibody only contains a humanized light chain. In some embodiments, a humanized antibody only contains a humanized heavy chain. In some embodiments, a humanized antibody only contains a humanized variable domain of a light chain and/or humanized variable domain of a heavy chain. In some embodiments, a humanized antibody contains a light chain as well as at least the variable domain of a heavy chain. In some embodiments, a humanized antibody contains a heavy chain as well as at least the variable domain of a light chain.

The terms “dual variable domain (DVD) binding protein” and “dual variable domain immunoglobulin” refer to a binding protein that has at least two variable domains in each of its one or more binding arms (e.g., a pair of HC/LC) (see PCT Publication No. WO 02/02773). Each variable domain is able to bind to an antigen/ligand. In an embodiment, each variable domain binds different antigens/ligands or epitopes. In another embodiment, each variable domain binds the same antigen/ligand or epitope. In another embodiment, a dual variable domain binding protein has two identical antigen/ligand binding arms, with identical specificity and identical VD sequences, and is bivalent for each antigen to which it binds. In an embodiment, the DVD binding proteins may be monospecific, i.e., capable of binding one antigen/ligand or multispecific, i.e., capable of binding two or more antigens/ligands. DVD binding proteins comprising two heavy chain DVD polypeptides and two light chain DVD polypeptides are referred to as a DVD-Ig™. In an embodiment, each half of a four chain DVD binding protein comprises a heavy chain DVD polypeptide, and a light chain DVD polypeptide, and two variable domain binding sites. In an embodiment, each binding site comprises a heavy chain variable domain and a light chain variable domain with a total of 6 CDRs involved in antigen binding per antigen binding site. In a specific embodiment of the present invention, at least one binding site comprises a receptor binding site, capable of binding one or more receptor ligands.

The term “antiidiotypic antibody” refers to an antibody raised against the amino acid sequence of the antigen combining site of another antibody. Antiidiotypic antibodies may be administered to enhance an immune response against an antigen.

The terms “parent antibody”, “parent receptor”, or more generically, “parent binding protein” refer to a pre-existing, or previously isolated binding protein from which a functional binding domain is utilized in a novel binding protein construct.

The term “biological activity” refers to any one or more biological properties of a molecule (whether present naturally as found in vivo, or provided or enabled by recombinant means). Biological properties include, but are not limited to, binding a receptor or receptor ligand, inducing cell proliferation, inhibiting cell growth, inducing other cytokines, inducing apoptosis, and enzymatic activity.

The term “neutralizing” refers to counteracting the biological activity of an antigen/ligand when a binding protein specifically binds to the antigen/ligand. In an embodiment, the neutralizing binding protein binds to an antigen/ligand (e.g., a cytokine) and reduces its biologically activity by at least about 20%, 40%, 60%, 80%, 85% or more.

“Specificity” refers to the ability of a binding protein to selectively bind an antigen/ligand.

“Affinity” is the strength of the interaction between a binding protein and an antigen/ligand, and is determined by the sequence of the binding domain(s) of the binding protein as well as by the nature of the antigen/ligand, such as its size, shape, and/or charge. Binding proteins may be selected for affinities that provide desired therapeutic end-points while minimizing negative side-effects. Affinity may be measured using methods known to one skilled in the art (US 20090311253).

The term “potency” refers to the ability of a binding protein to achieve a desired effect, and is a measurement of its therapeutic efficacy. Potency may be assessed using methods known to one skilled in the art (US 20090311253).

The term “cross-reactivity” refers to the ability of a binding protein to bind a target other than that against which it was raised. Generally, a binding protein will bind its target tissue(s)/antigen(s) with an appropriately high affinity, but will display an appropriately low affinity for non-target normal tissues. Individual binding proteins are generally selected to meet two criteria. (1) Tissue staining appropriate for the known expression of the antibody target. (2) Similar staining pattern between human and tox species (mouse and cynomolgus monkey) tissues from the same organ. These and other methods of assessing cross-reactivity are known to one skilled in the art (US 20090311253).

The term “biological function” refers the specific in vitro or in vivo actions of a binding protein. Binding proteins may target several classes of antigens/ligands and achieve desired therapeutic outcomes through multiple mechanisms of action. Binding proteins may target soluble proteins, cell surface antigens, as well as extracellular protein deposits. Binding proteins may agonize, antagonize, or neutralize the activity of their targets. Binding proteins may assist in the clearance of the targets to which they bind, or may result in cytotoxicity when bound to cells. Portions of two or more antibodies may be incorporated into a multivalent format to achieve distinct functions in a single binding protein molecule. The in vitro assays and in vivo models used to assess biological function are known to one skilled in the art (US 20090311253).

A “stable” binding protein is one in which the binding protein essentially retains its physical stability, chemical stability and/or biological activity upon storage. A multivalent binding protein that is stable in vitro at various temperatures for an extended period of time is desirable. Methods of stabilizing binding proteins and assessing their stability at various temperatures are known to one skilled in the art (US 20090311253).

The term “solubility” refers to the ability of a protein to remain dispersed within an aqueous solution. The solubility of a protein in an aqueous formulation depends upon the proper distribution of hydrophobic and hydrophilic amino acid residues, and therefore, solubility can correlate with the production of correctly folded proteins. A person skilled in the art will be able to detect an increase or decrease in solubility of a binding protein using routine HPLC techniques and methods known to one skilled in the art (US 20090311253).

Binding proteins may be produced using a variety of host cells or may be produced in vitro, and the relative yield per effort determines the “production efficiency.” Factors influencing production efficiency include, but are not limited to, host cell type (prokaryotic or eukaryotic), choice of expression vector, choice of nucleotide sequence, and methods employed. The materials and methods used in binding protein production, as well as the measurement of production efficiency, are known to one skilled in the art (US 20090311253).

The term “immunogenicity” means the ability of a substance to induce an immune response. Administration of a therapeutic binding protein may result in a certain incidence of an immune response. Potential elements that might induce immunogenicity in a multivalent format may be analyzed during selection of the parental binding proteins, and steps to reduce such risk can be taken to optimize the parental binding proteins prior to incorporating their sequences into a multivalent binding protein format. Methods of reducing the immunogenicity of antibodies and binding proteins are known to one skilled in the art (e.g., US 20090311253).

The terms “label” and “detectable label” mean a moiety attached to a member of a specific binding pair, such as an antibody or its analyte to render a reaction (e.g., binding) between the members of the specific binding pair, detectable. The labeled member of the specific binding pair is referred to as “detectably labeled.” Thus, the term “labeled binding protein” refers to a protein with a label incorporated that provides for the identification of the binding protein. In an embodiment, the label is a detectable marker that can produce a signal that is detectable by visual or instrumental means, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides (e.g., ³H, ¹⁴C, ³⁵S, ⁹⁹Y, ⁹⁹Tc, ¹¹¹In, ¹²⁵I, ¹³¹I, ¹⁷⁷Lu, ¹⁶⁶Ho, or ¹⁵³Sm); chromogens, fluorescent labels (e.g., FITC, rhodamine, lanthanide phosphors), enzymatic labels (e.g., horseradish peroxidase, luciferase, alkaline phosphatase); chemiluminescent markers; biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates. Representative examples of labels commonly employed for immunoassays include moieties that produce light, e.g., acridinium compounds, and moieties that produce fluorescence, e.g., fluorescein. In this regard, the moiety itself may not be detectably labeled but may become detectable upon reaction with yet another moiety.

The term “conjugate” refers to a binding protein, such as an antibody, that is chemically linked to a second chemical moiety, such as a therapeutic or cytotoxic agent. The term “agent” includes a chemical compound, a mixture of chemical compounds, a biological macromolecule, or an extract made from biological materials. In an embodiment, the therapeutic or cytotoxic agents include, but are not limited to, pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. When employed in the context of an immunoassay, the conjugate antibody may be a detectably labeled antibody used as the detection antibody.

The terms “crystal” and “crystallized” refer to a binding protein (e.g., an antibody), or antigen binding portion thereof, that exists in the form of a crystal. Crystals are one form of the solid state of matter, which is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well-understood in the field. The fundamental unit, or building block, that is repeated in a crystal is called the asymmetric unit. Repetition of the asymmetric unit in an arrangement that conforms to a given, well-defined crystallographic symmetry provides the “unit cell” of the crystal. Repetition of the unit cell by regular translations in all three dimensions provides the crystal. See Giege, R. and Ducruix, A. Barrett, CRYSTALLIZATION OF NUCLEIC ACIDS AND PROTEINS, A PRACTICAL APPROACH, 2nd ea., pp. 20 1-16, Oxford University Press, New York, N.Y., (1999).

The term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Other vectors include RNA vectors. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, other forms of expression vectors are also included, such as viral vectors (e.g., replication defective retroviruses, adenoviruses and adeno-associated viruses), which serve equivalent functions. A group of pHybE vectors (U.S. Patent Application Ser. No. 61/021,282) were used for cloning.

The terms “recombinant host cell” or “host cell” refer to a cell into which exogenous DNA has been introduced. Such terms refer not only to the particular subject cell, but to the progeny of such a cell. Because certain modifications may occur in succeeding generations due to either mutation or environmental influences, such progeny may not, in fact, be identical to the parent cell, but are still included within the scope of the term “host cell” as used herein. In an embodiment, host cells include prokaryotic and eukaryotic cells. In an embodiment, eukaryotic cells include protist, fungal, plant and animal cells. In another embodiment, host cells include but are not limited to the prokaryotic cell line E. Coli; mammalian cell lines CHO, HEK293, COS, NS0, SP2 and PER.C6; the insect cell line Sf9; and the fungal cell Saccharomyces cerevisiae.

The term “transfection” encompasses a variety of techniques commonly used for the introduction of exogenous nucleic acid (e.g., DNA) into a host cell, e.g., electroporation, calcium-phosphate precipitation, DEAE-dextran transfection and the like.

The term “cytokine” refers to a protein released by one cell population that acts on another cell population as an intercellular mediator. The term “cytokine” includes proteins from natural sources or from recombinant cell culture and biologically active equivalents of the native sequence cytokines.

The term “biological sample” means a quantity of a substance from a living thing or formerly living thing. Such substances include, but are not limited to, blood, (e.g., whole blood), plasma, serum, urine, amniotic fluid, synovial fluid, endothelial cells, leukocytes, monocytes, other cells, organs, tissues, bone marrow, lymph nodes and spleen.

The term “component” refers to an element of a composition. In relation to a diagnostic kit, for example, a component may be a capture antibody, a detection or conjugate antibody, a control, a calibrator, a series of calibrators, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample. Thus, a “component” can include a polypeptide or other analyte as above, that is immobilized on a solid support, such as by binding to an anti-analyte (e.g., anti-polypeptide) antibody. Some components can be in solution or lyophilized for reconstitution for use in an assay.

“Control” refers to a composition known to not analyte (“negative control”) or to contain analyte (“positive control”). A positive control can comprise a known concentration of analyte. “Control,” “positive control,” and “calibrator” may be used interchangeably herein to refer to a composition comprising a known concentration of analyte. A “positive control” can be used to establish assay performance characteristics and is a useful indicator of the integrity of reagents (e.g., analytes).

“Predetermined cutoff” and “predetermined level” refer generally to an assay cutoff value that is used to assess diagnostic/prognostic/therapeutic efficacy results by comparing the assay results against the predetermined cutoff/level, where the predetermined cutoff/level already has been linked or associated with various clinical parameters (e.g., severity of disease, progression/nonprogression/improvement, etc.). While the present disclosure may provide exemplary predetermined levels, it is well-known that cutoff values may vary depending on the nature of the immunoassay (e.g., antibodies employed, etc.). It further is well within the ordinary skill of one in the art to adapt the disclosure herein for other immunoassays to obtain immunoassay-specific cutoff values for those other immunoassays based on this disclosure. Whereas the precise value of the predetermined cutoff/level may vary between assays, correlations as described herein (if any) may be generally applicable.

“Pretreatment reagent,” e.g., lysis, precipitation and/or solubilization reagent, as used in a diagnostic assay as described herein is one that lyses any cells and/or solubilizes any analyte that is/are present in a test sample. Pretreatment is not necessary for all samples, as described further herein. Among other things, solubilizing the analyte (e.g., polypeptide of interest) may entail release of the analyte from any endogenous binding proteins present in the sample. A pretreatment reagent may be homogeneous (not requiring a separation step) or heterogeneous (requiring a separation step). With use of a heterogeneous pretreatment reagent there is removal of any precipitated analyte binding proteins from the test sample prior to proceeding to the next step of the assay.

“Quality control reagents” in the context of immunoassays and kits described herein, include, but are not limited to, calibrators, controls, and sensitivity panels. A “calibrator” or “standard” typically is used (e.g., one or more, such as a plurality) in order to establish calibration (standard) curves for interpolation of the concentration of an analyte, such as an antibody or an analyte. Alternatively, a single calibrator, which is near a predetermined positive/negative cutoff, can be used. Multiple calibrators (i.e., more than one calibrator or a varying amount of calibrator(s)) can be used in conjunction so as to comprise a “sensitivity panel.”

The term “specific binding partner” refers to a member of a specific binding pair. A specific binding pair comprises two different molecules that specifically bind to each other through chemical or physical means. Therefore, in addition to antigen and antibody specific binding, other specific binding pairs can include biotin and avidin (or streptavidin), carbohydrates and lectins, complementary nucleotide sequences, effector and receptor molecules, cofactors and enzymes, enzyme inhibitors and enzymes, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes, fragments, and variants (including fragments of variants) thereof, whether isolated or recombinantly produced.

The term “Fc region” defines the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a CH2 domain and a CH3 domain, and optionally comprises a CH4 domain Replacements of amino acid residues in the Fc portion to alter antibody effector function are known in the art (e.g., U.S. Pat. Nos. 5,648,260 and 5,624,821). The Fc region mediates several important effector functions, e.g., cytokine induction, antibody dependent cell mediated cytotoxicity (ADCC), phagocytosis, complement dependent cytotoxicity (CDC), and half-life/clearance rate of antibody and antigen-antibody complexes. In some cases these effector functions are desirable for a therapeutic immunoglobulin but in other cases might be unnecessary or even deleterious, depending on the therapeutic objectives.

The term “antigen-binding portion” of a binding protein means one or more fragments of a binding protein (preferrably, an antibody, or a receptor) that retain the ability to specifically bind to an antigen. The antigen-binding portion of a binding protein can be performed by fragments of a full-length antibody, as well as bispecific, dual specific, or multi-specific formats; specifically binding to two or more different antigens. Examples of binding fragments encompassed within the term “antigen-binding portion” of an binding protein include (i) an Fab fragment, a monovalent fragment consisting of the VL, VH, CL and CH1 domains; (ii) an F(ab′)₂ fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment, which comprises a single variable domain; and (vi) an isolated complementarity determining region (CDR). Furthermore, although the two domains of the Fv fragment, VL and VH, encoded by separate genes, they can be joined, using recombinant methods, by a synthetic linker that enables them to be made as a single protein chain in which the VL and VH regions pair to form monovalent molecules (known as single chain Fv (scFv). Such single chain antibodies are also intended to be encompassed within the term “antigen-binding portion” of an antibody. Other forms of single chain antibodies, such as diabodies are also encompassed. In addition, single chain antibodies also include “linear antibodies” comprising a pair of tandem Fv segments (VH-CH1-VH-CH1) which, together with complementary light chain polypeptides, form a pair of antigen binding regions.

The term “monovalent binding protein” refers to a binding protein comprising one antigen (ligand) binding site for each antigen. The term “multivalent binding protein” means a binding protein comprising two or more antigen (ligand) binding sites for the same antigen. In an embodiment, the multivalent binding protein is engineered to have three or more antigen binding sites, and is not a naturally occurring antibody. The term “multispecific binding protein” refers to a binding protein capable of binding two or more related or unrelated targets. In an embodiment, a monovalent binding proteins may be multispecific in that it possess one binding domain for each of the different target antigens.

The term “linker” means an amino acid residue or a polypeptide comprising two or more amino acid residues joined by peptide bonds that are used to link two polypeptides (e.g., two VH or two VL domains) Such linker polypeptides are well known in the art (see, e.g., Holliger et al. (1993) Proc. Natl. Acad. Sci. USA 90:6444-6448; Poljak et al. (1994) Structure 2:1121-1123).

The terms “Kabat numbering”, “Kabat definitions” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion thereof (Kabat et al. (1971) Ann NY Acad. Sci. 190:382-391 and, Kabat et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

The term “CDR” means a complementarity determining region within an immunoglobulin variable region sequence. There are three CDRs in each of the variable regions of the heavy chain and the light chain, which are designated CDR1, CDR2 and CDR3, for each of the heavy and light chain variable regions. The term “CDR set” refers to a group of three CDRs that occur in a single variable region capable of binding the antigen. The exact boundaries of these CDRs have been defined differently according to different systems. The system described by Kabat (Kabat et al. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia and Lesk (1987) J. Mol. Biol. 196:901-917; Chothia et al. (1989) Nature 342:877-883) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan (1995) FASEB J. 9:133-139 and MacCallum (1996) J. Mol. Biol. 262(5):732-45). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat or Chothia defined CDRs.

The term “epitope” means a region of an antigen that is bound by a binding protein, e.g., a polypeptide and/or other determinant capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. In an embodiment, an epitope comprises the amino acid residues of a region of an antigen (or fragment thereof) known to bind to the complementary site on the specific binding partner. An antigenic fragment can contain more than one epitope. In certain embodiments, a binding protein specifically binds an antigen when it recognizes its target antigen in a complex mixture of proteins and/or macromolecules. Binding proteins “bind to the same epitope” if the antibodies cross-compete (one prevents the binding or modulating effect of the other). In addition, structural definitions of epitopes (overlapping, similar, identical) are informative; and functional definitions encompass structural (binding) and functional (modulation, competition) parameters. Different regions of proteins may perform different functions. For example specific regions of a cytokine interact with its cytokine receptor to bring about receptor activation whereas other regions of the protein may be required for stabilizing the cytokine. To abrogate the negative effects of cytokine signaling, the cytokine may be targeted with a binding protein that binds specifically to the receptor interacting region(s), thereby preventing the binding of its receptor. Alternatively, a binding protein may target the regions responsible for cytokine stabilization, thereby designating the protein for degradation. The methods of visualizing and modeling epitope recognition are known to one skilled in the art (US 20090311253).

“Pharmacokinetics” refers to the process by which a drug is absorbed, distributed, metabolized, and excreted by an organism. To generate a multivalent binding protein molecule with a desired pharmacokinetic profile, parent binding proteins with similarly desired pharmacokinetic profiles are selected. The PK profiles of the selected parental binding proteins can be easily determined in rodents using methods known to one skilled in the art (US 20090311253).

“Bioavailability” refers to the amount of active drug that reaches its target following administration. Bioavailability is function of several of the previously described properties, including stability, solubility, immunogenicity and pharmacokinetics, and can be assessed using methods known to one skilled in the art (US 20090311253).

The term “surface plasmon resonance” means an optical phenomenon that allows for the analysis of real-time biospecific interactions by detection of alterations in protein concentrations within a biosensor matrix, for example using the BIAcore® system (BIAcore International AB, a GE Healthcare company, Uppsala, Sweden and Piscataway, N.J.). For further descriptions, see Jönsson et al. (1993) Ann Biol. Clin. 51:19-26. The term “Kₒₙ” means the on rate constant for association of a binding protein (e.g., an antibody or DVD-Ig) to the antigen to form the, e.g., DVD-Ig/antigen complex. The term “Kₒₙ” also means “association rate constant”, or “ka”, as is used interchangeably herein. This value indicating the binding rate of a binding protein to its target antigen or the rate of complex formation between a binding protein, e.g., an antibody, and antigen also is shown by the equation below:

Antibody (“Ab”)+Antigen (“Ag”)→Ab-Ag

The term “K_{off}” means the off rate constant for dissociation, or “dissociation rate constant”, of a binding protein (e.g., an antibody or DVD-Ig) from the, e.g., DVD-Ig/antigen complex as is known in the art. This value indicates the dissociation rate of a binding protein, e.g., an antibody, from its target antigen or separation of Ab-Ag complex over time into free antibody and antigen as shown by the equation below:

Ab+Ag←Ab-Ag

The terms “K_{d}” and “equilibrium dissociation constant” means the value obtained in a titration measurement at equilibrium, or by dividing the dissociation rate constant (K_{off}) by the association rate constant (Kₒₙ). The association rate constant, the dissociation rate constant and the equilibrium dissociation constant, are used to represent the binding affinity of a binding protein (e.g., an antibody or DVD-Ig) to an antigen. Methods for determining association and dissociation rate constants are well known in the art. Using fluorescence-based techniques offers high sensitivity and the ability to examine samples in physiological buffers at equilibrium. Other experimental approaches and instruments such as a BIAcore® (biomolecular interaction analysis) assay, can be used (e.g., instrument available from BIAcore International AB, a GE Healthcare company, Uppsala, Sweden). Additionally, a KinExA® (Kinetic Exclusion Assay) assay, available from Sapidyne Instruments (Boise, Id.), can also be used.

The term “variant” means a polypeptide that differs from a given polypeptide in amino acid sequence by the addition (e.g., insertion), deletion, or conservative substitution of amino acids, but that retains the biological activity of the given polypeptide (e.g., a variant IL-17 antibody can compete with anti-IL-17 antibody for binding to IL-17). A conservative substitution of an amino acid, i.e., replacing an amino acid with a different amino acid of similar properties (e.g., hydrophilicity and degree and distribution of charged regions) is recognized in the art as typically involving a minor change. These minor changes can be identified, in part, by considering the hydropathic index of amino acids, as understood in the art (see, e.g., Kyte et al. (1982) J. Mol. Biol. 157: 105-132). The hydropathic index of an amino acid is based on a consideration of its hydrophobicity and charge. It is known in the art that amino acids of similar hydropathic indexes in a protein can be substituted and the protein still retains protein function. In one aspect, amino acids having hydropathic indexes of ±2 are substituted. The hydrophilicity of amino acids also can be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity (see, e.g., U.S. Pat. No. 4,554,101). Substitution of amino acids having similar hydrophilicity values can result in peptides retaining biological activity, for example immunogenicity, as is understood in the art. In one aspect, substitutions are performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. The term “variant” also includes polypeptide or fragment thereof that has been differentially processed, such as by proteolysis, phosphorylation, or other post-translational modification, yet retains its biological activity or antigen reactivity, e.g., the ability to bind to IL-17. The term “variant” encompasses fragments of a variant unless otherwise defined. A variant may be 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, or 75% identical to the wildtype sequence.

The multi-specific binding proteins and methods of making the same are provided. The binding protein can be generated using various techniques. Expression vectors, host cells and methods of generating the binding proteins are provided in this disclosure.

The antigen-binding variable domains of the binding proteins of this disclosure can be obtained from parent binding proteins, including polyclonal Abs, monoclonal Abs, and or receptors capable of binding antigens of interest. These parent binding proteins may be naturally occurring or may be generated by recombinant technology. The person of ordinary skill in the art is well familiar with many methods for producing antibodies and/or isolated receptors, including, but not limited to using hybridoma techniques, selected lymphocyte antibody method (SLAM), use of a phage, yeast, or RNA-protein fusion display or other library, immunizing a non-human animal comprising at least some of the human immunoglobulin locus, and preparation of chimeric, CDR-grafted, and humanized antibodies. See, e.g., US Patent Publication No. 20090311253 A1. Variable domains may also be prepared using affinity maturation techniques. The binding variable domains of the binding proteins can also be obtained from isolated receptor molecules obtained by extraction procedures known in the art (e.g., using solvents, detergents, and/or affinity purifications), or determined by biophysical methods known in the art (e.g., X-ray crystallography, NMR, interferometry, and/or computer modeling).

An embodiment is provided comprising selecting parent binding proteins with at least one or more properties desired in the binding protein molecule. In an embodiment, the desired property is one or more of those used to characterize antibody parameters, such as, for example, antigen specificity, affinity to antigen, potency, biological function, epitope recognition, stability, solubility, production efficiency, immunogenicity, pharmacokinetics, bioavailability, tissue cross reactivity, or orthologous antigen binding. See, e.g., US Patent Publication No. 20090311253.

The multi-specific antibodies may also be designed such that one or more of the antigen binding domain are rendered non-functional. The variable domains may be obtained using recombinant DNA techniques from parent binding proteins generated by any one of the methods described herein. In an embodiment, a variable domain is a murine heavy or light chain variable domain. In another embodiment, a variable domain is a CDR grafted or a humanized variable heavy or light chain domain. In an embodiment, a variable domain is a human heavy or light chain variable domain.

The linker sequence may be a single amino acid or a polypeptide sequence. In an embodiment, the choice of linker sequences is based on crystal structure analysis of several Fab molecules. There is a natural flexible linkage between the variable domain and the CH1/CL constant domain in Fab or antibody molecular structure. This natural linkage may contain approximately 10-12 amino acid residues, contributed by 4-6 residues from the C-terminus of a V domain and 4-6 residues from the N-terminus of a CL/CH1 domain. The binding proteins may be generated using N-terminal 5-6 amino acid residues, or 11-12 amino acid residues, of CL or CH1 as a linker in the light chain and heavy chains, respectively. The N-terminal residues of CL or CH1 domains, particularly the first 5-6 amino acid residues, can adopt a loop conformation without strong secondary structures, and therefore can act as flexible linkers between the two variable domains. The N-terminal residues of CL or CH1 domains are natural extension of the variable domains, as they are part of the Ig sequences, and therefore their use may minimize to a large extent any immunogenicity potentially arising from the linkers and junctions.

Other linker sequences may include any sequence of any length of a CL/CH1 domain but not all residues of a CL/CH1 domain; for example the first 5-12 amino acid residues of a CL/CH1 domain; the light chain linkers can be from Cκ or Cλ; and the heavy chain linkers can be derived from CH1 of any isotype, including Cγ1, Cγ2, Cγ3, Cγ4, Cα1, Cα2, Cδ, Cε, and Cμ. Linker sequences may also be derived from other proteins such as Ig-like proteins (e.g., TCR, FcR, KIR); G/S based sequences (e.g., G4S repeats); hinge region-derived sequences; and other natural sequences from other proteins.

In an embodiment, one or more constant domains are linked to the variable domains using recombinant DNA techniques. In an embodiment, a sequence comprising one or more heavy chain variable domains is linked to a heavy chain constant domain and a sequence comprising one or more light chain variable domains is linked to a light chain constant domain. In an embodiment, the constant domains are human heavy chain constant domains and human light chain constant domains, respectively. In an embodiment, the heavy chain is further linked to an Fc region. The Fc region may be a native sequence Fc region or a variant Fc region. In another embodiment, the Fc region is a human Fc region. In another embodiment, the Fc region includes Fc region from IgG1, IgG2, IgG3, IgG4, IgA, IgM, IgE, or IgD.

Detailed description of specific binding proteins capable of binding specific targets, and methods of making the same, is provided in the Examples section below.

In one embodiment, at least 50%, at least 75% or at least 90% of the assembled, and immunoglobulin molecules expressed in a host cell are the desired VD cross-over binding proteins, and therefore possess enhanced commercial utility.

Methods of expressing a VD cross-over binding protein in a single cell leading to a “primary product” of a “multi-specific binding protein”, where the “primary product” is more than 50%, more than 75% or more than 90%, of all assembled protein are provided.

In an embodiment, the binding proteins provided herein are capable of neutralizing the activity of their antigen targets both in vitro and in vivo. Accordingly, such binding proteins can be used to inhibit antigen activity, e.g., in a cell culture containing the antigens, in human subjects or in other mammalian subjects having the antigens with which a binding protein provided herein cross-reacts. In another embodiment, a method for reducing antigen activity in a subject suffering from a disease or disorder in which the antigen activity is detrimental is provided. A binding protein provided herein can be administered to a human subject for therapeutic purposes.

The term “a disorder in which antigen activity is detrimental” is intended to include diseases and other disorders in which the presence of the antigen in a subject suffering from the disorder has been shown to be or is suspected of being either responsible for the pathophysiology of the disorder or a factor that contributes to a worsening of the disorder. Accordingly, a disorder in which antigen activity is detrimental is a disorder in which reduction of antigen activity is expected to alleviate the symptoms and/or progression of the disorder. Such disorders may be evidenced, for example, by an increase in the concentration of the antigen in a biological fluid of a subject suffering from the disorder (e.g., an increase in the concentration of antigen in serum, plasma, synovial fluid, etc., of the subject). Non-limiting examples of disorders that can be treated with the binding proteins provided herein include those disorders discussed below and in the section pertaining to pharmaceutical compositions comprising the binding proteins.

Additionally, the binding proteins provided herein can be employed for tissue-specific delivery (target a tissue marker and a disease mediator for enhanced local PK thus higher efficacy and/or lower toxicity), including intracellular delivery (targeting an internalizing receptor and an intracellular molecule), delivering to inside brain (targeting transferrin receptor and a CNS disease mediator for crossing the blood-brain barrier). The binding proteins can also serve as a carrier protein to deliver an antigen to a specific location via binding to a non-neutralizing epitope of that antigen and also to increase the half-life of the antigen. Furthermore, the binding proteins can be designed to either be physically linked to medical devices implanted into patients or target these medical devices (see Burke et al. (2006) Advanced Drug Deliv. Rev. 58(3): 437-446; Hildebrand et al. (2006) Surface and Coatings Technol. 200(22-23): 6318-6324; Drug/device combinations for local drug therapies and infection prophylaxis, Wu (2006) Biomaterials 27(11):2450-2467; Mediation of the cytokine network in the implantation of orthopedic devices, Marques (2005) Biodegradable Systems in Tissue Engineer. Regen. Med. 377-397). Directing appropriate types of cell to the site of medical implant may promote healing and restoring normal tissue function. Alternatively, inhibition of mediators (including but not limited to cytokines), released upon device implantation by a receptor antibody fusion protein coupled to or target to a device is also provided.

Binding protein molecules provided herein are useful as therapeutic molecules to treat various diseases, e.g., wherein the targets that are recognized by the binding proteins are detrimental. Such binding proteins may bind one or more targets involved in a specific disease.

Without limiting the disclosure, further information on certain disease conditions is provided.

### 1. Human Autoimmune and Inflammatory Response

Various cytokines and chemokines have been implicated in general autoimmune and inflammatory responses, including, for example, asthma, allergies, allergic lung disease, allergic rhinitis, atopic dermatitis, chronic obstructive pulmonary disease (COPD), fibrosis, cystic fibrosis (CF), fibrotic lung disease, idiopathic pulmonary fibrosis, liver fibrosis, lupus, hepatitis B-related liver diseases and fibrosis, sepsis, systemic lupus erythematosus (SLE), glomerulonephritis, inflammatory skin diseases, psoriasis, diabetes, insulin dependent diabetes mellitus, inflammatory bowel disease (IBD), ulcerative colitis (UC), Crohn's disease (CD), rheumatoid arthritis (RA), osteoarthritis (OA), multiple sclerosis (MS), graft-versus-host disease (GVHD), transplant rejection, ischemic heart disease (IHD), celiac disease, contact hypersensitivity, alcoholic liver disease, Behcet's disease, atherosclerotic vascular disease, occular surface inflammatory diseases, or Lyme disease.

The binding proteins provided herein can be used to treat neurological disorders. In an embodiment, the binding proteins provided herein or antigen-binding portions thereof, are used to treat neurodegenerative diseases, and conditions involving neuronal regeneration and spinal cord injury.

### 2. Asthma

Allergic asthma is characterized by the presence of eosinophilia, goblet cell metaplasia, epithelial cell alterations, airway hyperreactivity (AHR), and Th2 and Th1 cytokine expression, as well as elevated serum IgE levels. Corticosteroids are the most important anti-inflammatory treatment for asthma today, however their mechanism of action is non-specific and safety concerns exist, especially in the juvenile patient population. The development of more specific and targeted therapies is therefore warranted.

Various cytokines have been implicated as having a pivotal role in causing pathological responses associated with asthma. The development of mAb against these cotokines as well as rDVD-Ig™ constructs may prove effective in preventing and/or treating asthma.

Animal models such as an OVA-induced asthma mouse model, where both inflammation and AHR can be assessed, are known in the art and may be used to determine the ability of various binding protein molecules to treat asthma Animal models for studying asthma are disclosed in Coffman, et al. (2005) J. Exp. Med. 201(12):1875-1879; Lloyd et al. (2001) Adv. Immunol. 77: 263-295; Boyce et al. (2005) J. Exp. Med. 201(12):1869-1873; and Snibson et al. (2005) J. Brit. Soc. Allergy Clin. Immunol. 35(2):146-52. In addition to routine safety assessments of these target pairs specific tests for the degree of immunosuppression may be warranted and helpful in selecting the best target pairs (see Luster et al. (1994) Toxicol. 92(1-3):229-43; Descotes et al. (1992) Dev. Biol. Standard. 77:99-102; Hart et al. (2001) J. Allergy Clin. Immunol. 108(2):250-257).

### 3. Rheumatoid Arthritis

Rheumatoid arthritis (RA), a systemic disease, is characterized by a chronic inflammatory reaction in the synovium of joints and is associated with degeneration of cartilage and erosion of juxta-articular bone. Many pro-inflammatory cytokines, chemokines, and growth factors are expressed in diseased joints. Recent studies indicate that the involvement of T cells in RA is mediated to a significant extent by certain cytokines. Beneficial effects of blocking these cytokines were also observed various animal models of the disease (for a review see Witowski et al. (2004) Cell. Mol. Life. Sci. 61: 567-579). Whether a binding protein molecule will be useful for the treatment of rheumatoid arthritis can be assessed using pre-clinical animal RA models such as the collagen-induced arthritis mouse model. Other useful models are also well known in the art (see Brand (2005) Comp. Med. 55(2):114-22). Based on the cross-reactivity of the parental antibodies for human and mouse orthologues (e.g., reactivity for human and mouse TNF, human and mouse IL-15, etc.) validation studies in the mouse CIA model may be conducted with “matched surrogate antibody” derived binding protein molecules; briefly, a binding protein based on two (or more) mouse target specific antibodies may be matched to the extent possible to the characteristics of the parental human or humanized antibodies used for human binding protein construction (e.g., similar affinity, similar neutralization potency, similar half-life, etc.).

### 4. Systemic Lupus Erythematosus (SLE)

The immunopathogenic hallmark of SLE is the polyclonal B cell activation, which leads to hyperglobulinemia, autoantibody production and immune complex formation. Significant increased levels of certain cytokines have been detected in patients with systemic lupus erythematosus (Morimoto et al. (2001) Autoimmunity, 34(1):19-25; Wong et al. (2008) Clin Immunol. 127(3):385-93). Increased cytokine production has been shown in patients with SLE as well as in animals with lupus-like diseases. Animal models have demonstrated that blockade of these cytokines may decrease lupus manifestations (for a review see Nalbandian et al. (2009) 157(2): 209-215). Based on the cross-reactivity of the parental antibodies for human and mouse othologues (e.g., reactivity for human and mouse CD20, human and mouse interferon alpha, etc.) validation studies in a mouse lupus model may be conducted with “matched surrogate antibody” derived binding protein molecules. Briefly, a binding protein based two (or more) mouse target specific antibodies may be matched to the extent possible to the characteristics of the parental human or humanized antibodies used for human binding protein construction (e.g., similar affinity, similar neutralization potency, similar half-life, etc.).

### 5. Multiple Sclerosis

Multiple sclerosis (MS) is a complex human autoimmune-type disease with a predominantly unknown etiology. Immunologic destruction of myelin basic protein (MBP) throughout the nervous system is the major pathology of multiple sclerosis. Of major consideration are immunological mechanisms that contribute to the development of autoimmunity. In particular, antigen expression, cytokine and leukocyte interactions, and regulatory T-cells, which help balance/modulate other T-cells such as Th1 and Th2 cells, are important areas for therapeutic target identification. In MS, increased expression of certain cytokine has been detected both in brain lesions and in mononuclear cells isolated from blood and cerebrospinal fluid. Cells producing these cytokines are highly enriched in active MS lesions, suggesting that neutralization of this cytokine has the potential of being beneficial (for a review see Witowski et al. (2004) Cell. Mol. Life. Sci. 61: 567-579).

Several animal models for assessing the usefulness of the binding proteins to treat MS are known in the art (see Steinman et al. (2005) Trends Immunol. 26(11):565-71; Lublin et al. (1985) Springer Semin Immunopathol. 8(3):197-208; Genain et al. (1997) J. Mol. Med. 75(3):187-97; Tuohy et al. (1999) J. Exp. Med. 189(7):1033-42; Owens et al. (1995) Neurol. Clin. 13(1):51-73; and Hart et al. (2005) J. Immunol. 175(7):4761-8.) Based on the cross-reactivity of the parental antibodies for human and animal species othologues validation studies in the mouse EAE model may be conducted with “matched surrogate antibody” derived binding protein molecules. Briefly, a binding protein based on two (or more) mouse target specific antibodies may be matched to the extent possible to the characteristics of the parental human or humanized antibodies used for human binding protein construction (e.g., similar affinity, similar neutralization potency, similar half-life, etc.). The same concept applies to animal models in other non-rodent species, where a “matched surrogate antibody” derived binding protein would be selected for the anticipated pharmacology and possibly safety studies. In addition to routine safety assessments of these target pairs specific tests for the degree of immunosuppression may be warranted and helpful in selecting the best target pairs (see Luster et al. (1994) Toxicol. 92(1-3): 229-43; Descotes et al. (1992) Devel. Biol. Standard. 77: 99-102; Jones (2000) IDrugs 3(4):442-6).

### 6. Sepsis

Overwhelming inflammatory and immune responses are essential features of septic shock and play a central part in the pathogenesis of tissue damage, multiple organ failure, and death induced by sepsis. Cytokines have been shown to be mediators of septic shock. These cytokines have a direct toxic effect on tissues; they also activate phospholipase A2. These and other effects lead to increased concentrations of platelet-activating factor, promotion of nitric oxide synthase activity, promotion of tissue infiltration by neutrophils, and promotion of neutrophil activity. The levels of certain cytokines and clinical prognosis of sepsis have been shown to be negatively correlated. Neutralization of antibody or rDVD-Ig™ constructs against these cytokines may significantly improve the survival rate of patients with sepsis (see Flierl et al. (2008) FASEB J. 22: 2198-2205).

One embodiment pertains to rDVD-Ig™ constructs capable of binding one or more targets involved in sepsis, such as, for example cytokines. The efficacy of such binding proteins for treating sepsis can be assessed in preclinical animal models known in the art (see Buras et al. (2005) Nat. Rev. Drug Discov. 4(10):854-65 and Calandra et al. (2000) Nat. Med. 6(2):164-70).

### 7. Neurological Disorders

a. Neurodegenerative Diseases

Neurodegenerative diseases are either chronic in which case they are usually age-dependent or acute (e.g., stroke, traumatic brain injury, spinal cord injury, etc.). They are characterized by progressive loss of neuronal functions (e.g., neuronal cell death, axon loss, neuritic dystrophy, demyelination), loss of mobility and loss of memory. These chronic neurodegenerative diseases represent a complex interaction between multiple cell types and mediators. Treatment strategies for such diseases are limited and mostly constitute either blocking inflammatory processes with non-specific anti-inflammatory agents (e.g., corticosteroids, COX inhibitors) or agents to prevent neuron loss and/or synaptic functions. These treatments fail to stop disease progression. Specific therapies targeting more than one disease mediator may provide even better therapeutic efficacy for chronic neurodegenerative diseases than observed with targeting a single disease mechanism (see Deane et al. (2003) Nature Med. 9:907-13; and Masliah et al. (2005) Neuron. 46:857).

The binding protein molecules provided herein can bind one or more targets involved in chronic neurodegenerative diseases such as Alzheimers. The efficacy of binding protein molecules can be validated in pre-clinical animal models such as the transgenic mice that over-express amyloid precursor protein or RAGE and develop Alzheimer's disease-like symptoms. In addition, binding protein molecules can be constructed and tested for efficacy in the animal models and the best therapeutic binding protein can be selected for testing in human patients. Binding protein molecules can also be employed for treatment of other neurodegenerative diseases such as Parkinson's disease.

b. Neuronal Regeneration and Spinal Cord Injury

Despite an increase in knowledge of the pathologic mechanisms, spinal cord injury (SCI) is still a devastating condition and represents a medical indication characterized by a high medical need. Most spinal cord injuries are contusion or compression injuries and the primary injury is usually followed by secondary injury mechanisms (inflammatory mediators e.g., cytokines and chemokines) that worsen the initial injury and result in significant enlargement of the lesion area, sometimes more than 10-fold. Certain cytokine is a mediator of secondary degeneration, which contributes to neuroinflammation and hinders functional recovery.

The efficacy of binding protein molecules can be validated in pre-clinical animal models of spinal cord injury. In addition, these binding protein molecules can be constructed and tested for efficacy in the animal models and the best therapeutic binding protein can be selected for testing in human patients. In general, antibodies do not cross the blood brain barrier (BBB) in an efficient and relevant manner. However, in certain neurologic diseases, e.g., stroke, traumatic brain injury, multiple sclerosis, etc., the BBB may be compromised and allows for increased penetration of binding proteins and antibodies into the brain. In other neurological conditions, where BBB leakage is not occurring, one may employ the targeting of endogenous transport systems, including carrier-mediated transporters such as glucose and amino acid carriers and receptor-mediated transcytosis-mediating cell structures/receptors at the vascular endothelium of the BBB, thus enabling trans-BBB transport of the binding protein. Structures at the BBB enabling such transport include but are not limited to the insulin receptor, transferrin receptor, LRP and RAGE. In addition, strategies enable the use of binding proteins also as shuttles to transport potential drugs into the CNS including low molecular weight drugs, nanoparticles and nucleic acids (Coloma et al. (2000) Pharm Res. 17(3):266-74; Boado et al. (2007) Bioconjug. Chem. 18(2):447-55).

### 8. Oncological Disorders

Monoclonal antibody therapy has emerged as an important therapeutic modality for cancer (von Mehren et al. (2003) Annu. Rev. Med. 54:343-69). Certain cytokines have been suggested to support tumor growth, probably by stimulating angiogenesis or by modulating anti-tumor immunity and tumor growth. Studies indicate that some cytokines may be central to the novel immunoregulatory pathway in which NKT cells suppress tumor immunosurveillance (For a review see Kolls et al. (2003) Am. J. Respir. Cell Mol. Biol. 28: 9-11, and Terabe et al. (2004) Cancer Immunol Immunother. 53(2):79-85.)

In an embodiment, diseases that can be treated or diagnosed with the compositions and methods provided herein include, but are not limited to, primary and metastatic cancers, including carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract (including cervix, uterus, and ovaries as well as choriocarcinoma and gestational trophoblastic disease), male genital tract (including prostate, seminal vesicles, testes and germ cell tumors), endocrine glands (including the thyroid, adrenal, and pituitary glands), and skin, as well as hemangiomas, melanomas, sarcomas (including those arising from bone and soft tissues as well as Kaposi's sarcoma), tumors of the brain, nerves, eyes, and meninges (including astrocytomas, gliomas, glioblastomas, retinoblastomas, neuromas, neuroblastomas, Schwannomas, and meningiomas), solid tumors arising from hematopoietic malignancies such as leukemias, and lymphomas (both Hodgkin's and non-Hodgkin's lymphomas).

In an embodiment, the antibodies provided herein or antigen-binding portions thereof, are used to treat cancer or in the prevention of metastases from the tumors described herein either when used alone or in combination with radiotherapy and/or other chemotherapeutic agents.

### 9. Gene Therapy

In a specific embodiment, nucleic acid sequences encoding a binding protein provided herein or another prophylactic or therapeutic agent provided herein are administered to treat, prevent, manage, or ameliorate a disorder or one or more symptoms thereof by way of gene therapy. Gene therapy refers to therapy performed by the administration to a subject of an expressed or expressible nucleic acid. In this embodiment, the nucleic acids produce their encoded antibody or prophylactic or therapeutic agent provided herein that mediates a prophylactic or therapeutic effect.

Any of the methods for gene therapy available in the art can be used in the methods provided herein. For general reviews of the methods of gene therapy, see Goldspiel et al. (1993) Clin. Pharmacy 12:488-505; Wu and Wu (1991) Biotherapy 3:87-95; Tolstoshev (1993) Ann Rev. Pharmacol. Toxicol. 32:573-596; Mulligan (1993) Science 260:926-932; Morgan and Anderson (1993) Ann Rev. Biochem. 62:191-217; and May (1993) TIBTECH 11(5):155-215. Methods commonly known in the art of recombinant DNA technology which can be used are described in Ausubel et al. (eds.), Current Protocols in Molecular Biology, John Wiley &Sons, NY (1993); and Kriegler, Gene Transfer and Expression, A Laboratory Manual, Stockton Press, NY (1990). Detailed description of various methods of gene therapy are disclosed in US Patent Publication No. US20050042664.

### II. Pharmaceutical Compositions

Pharmaceutical compositions comprising one or more binding proteins, either alone or in combination with prophylactic agents, therapeutic agents, and/or pharmaceutically acceptable carriers are provided. The pharmaceutical compositions comprising binding proteins provided herein are for use in, but not limited to, diagnosing, detecting, or monitoring a disorder, in preventing, treating, managing, or ameliorating a disorder or one or more symptoms thereof, and/or in research. The formulation of pharmaceutical compositions, either alone or in combination with prophylactic agents, therapeutic agents, and/or pharmaceutically acceptable carriers, are known to one skilled in the art (US Patent Publication No. 20090311253 A1).

Methods of administering a prophylactic or therapeutic agent provided herein include, but are not limited to, parenteral administration (e.g., intradermal, intramuscular, intraperitoneal, intravenous and subcutaneous), epidural administration, intratumoral administration, mucosal administration (e.g., intranasal and oral routes) and pulmonary administration (e.g., aerosolized compounds administered with an inhaler or nebulizer). The formulation of pharmaceutical compositions for specific routes of administration, and the materials and techniques necessary for the various methods of administration are available and known to one skilled in the art (US Patent Publication No. 20090311253 A1).

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. The term “dosage unit form” refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms provided herein are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of a binding protein provided herein is 0.1-20 mg/kg, for example, 1-10 mg/kg. It is to be noted that dosage values may vary with the type and severity of the condition to be alleviated. It is to be further understood that for any particular subject, specific dosage regimens may be adjusted over time according to the individual need and the professional judgment of the person administering or supervising the administration of the compositions, and that dosage ranges set forth herein are exemplary only and are not intended to limit the scope or practice of the claimed composition.

### III. Combination Therapy

A binding protein provided herein also can also be administered with one or more additional therapeutic agents useful in the treatment of various diseases, the additional agent being selected by the skilled artisan for its intended purpose. For example, the additional agent can be a therapeutic agent art-recognized as being useful to treat the disease or condition being treated by the antibody provided herein. The combination can also include more than one additional agent, e.g., two or three additional agents.

Combination therapy agents include, but are not limited to, antineoplastic agents, radiotherapy, chemotherapy such as DNA alkylating agents, cisplatin, carboplatin, anti-tubulin agents, paclitaxel, docetaxel, taxol, doxorubicin, gemcitabine, gemzar, anthracyclines, adriamycin, topoisomerase I inhibitors, topoisomerase II inhibitors, 5-fluorouracil (5-FU), leucovorin, irinotecan, receptor tyrosine kinase inhibitors (e.g., erlotinib, gefitinib), COX-2 inhibitors (e.g., celecoxib), kinase inhibitors, and siRNAs.

Combinations to treat autoimmune and inflammatory diseases are non-steroidal anti-inflammatory drug(s) also referred to as NSAIDS which include drugs like ibuprofen. Other combinations are corticosteroids including prednisolone; the well known side-effects of steroid use can be reduced or even eliminated by tapering the steroid dose required when treating patients in combination with the binding proteins provided herein. Non-limiting examples of therapeutic agents for rheumatoid arthritis with which an antibody provided herein, or antibody binding portion thereof, can be combined include the following: cytokine suppressive anti-inflammatory drug(s) (CSAIDs); antibodies to or antagonists of other human cytokines or growth factors, for example, TNF, LT, IL-1, IL-2, IL-3, IL-4, IL-5, IL-6, IL-7, IL-8, IL-15, IL-16, IL-18, IL-21, IL-23, interferons, EMAP-II, GM-CSF, FGF, and PDGF. Binding proteins provided herein, or antigen binding portions thereof, can be combined with antibodies to cell surface molecules such as CD2, CD3, CD4, CD8, CD25, CD28, CD30, CD40, CD45, CD69, CD80 (B7.1), CD86 (B7.2), CD90, CTLA or their ligands including CD154 (gp39 or CD40L).

Combinations of therapeutic agents may interfere at different points in the autoimmune and subsequent inflammatory cascade. Examples include a binding protein disclosed herein and a TNF antagonist like a chimeric, humanized or human TNF antibody, Adalimumab, (PCT Publication No. WO 97/29131), CA2 (Remicade™), CDP 571, a soluble p55 or p75 TNF receptor, or derivative thereof (p75TNFR1gG (Enbrel™) or p55TNFR1gG (Lenercept)), a TNFα converting enzyme (TACE) inhibitor; or an IL-1 inhibitor (an Interleukin-1-converting enzyme inhibitor, IL-1RA, etc.). Other combinations include a binding protein disclosed herein and Interleukin 11. Yet another combination include key players of the autoimmune response which may act parallel to, dependent on or in concert with IL-12 function; especially relevant are IL-18 antagonists including an IL-18 antibody, a soluble IL-18 receptor, or an IL-18 binding protein. It has been shown that IL-12 and IL-18 have overlapping but distinct functions and a combination of antagonists to both may be most effective. Yet another combination is a binding protein disclosed herein and a non-depleting anti-CD4 inhibitor. Yet other combinations include a binding protein disclosed herein and an antagonist of the co-stimulatory pathway CD80 (B7.1) or CD86 (B7.2) including an antibody, a soluble receptor, or an antagonistic ligand.

The binding proteins provided herein may also be combined with an agent, such as methotrexate, 6-MP, azathioprine sulphasalazine, mesalazine, olsalazine chloroquinine/hydroxychloroquine, pencillamine, aurothiomalate (intramuscular and oral), azathioprine, cochicine, a corticosteroid (oral, inhaled and local injection), a beta-2 adrenoreceptor agonist (salbutamol, terbutaline, salmeteral), a xanthine (theophylline, aminophylline), cromoglycate, nedocromil, ketotifen, ipratropium, oxitropium, cyclosporin, FK506, rapamycin, mycophenolate mofetil, leflunomide, an NSAID, for example, ibuprofen, a corticosteroid such as prednisolone, a phosphodiesterase inhibitor, an adensosine agonist, an antithrombotic agent, a complement inhibitor, an adrenergic agent, an agent which interferes with signalling by proinflammatory cytokines such as TNF-α or IL-1 (e.g., IRAK, NIK, IKK, p38 or a MAP kinase inhibitor), an IL-1β converting enzyme inhibitor, a TNFα converting enzyme (TACE) inhibitor, a T-cell signaling inhibitor such as a kinase inhibitor, a metalloproteinase inhibitor, sulfasalazine, azathioprine, a 6-mercaptopurine, an angiotensin converting enzyme inhibitor, a soluble cytokine receptor or derivative thereof (e.g., a soluble p55 or p75 TNF receptor or the derivative p75TNFRIgG (Enbrel™) or p55TNFRIgG (Lenercept), sIL-1RI, sIL-1RII, sIL-6R), an antiinflammatory cytokine (e.g., IL-4, IL-10, IL-11, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone hcl, hydrocodone bitartrate/apap, diclofenac sodium/misoprostol, fentanyl, anakinra, human recombinant, tramadol hcl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline hcl, sulfadiazine, oxycodone hcl/acetaminophen, olopatadine hcl, misoprostol, naproxen sodium, omeprazole, cyclophosphamide, rituximab, IL-1 TRAP, MRA, CTLA4-IG, IL-18 BP, anti-IL-18, Anti-IL15, BIRB-796, SC10-469, VX-702, AMG-548, VX-740, Roflumilast, IC-485, CDC-801, or Mesopram. Combinations include methotrexate or leflunomide and in moderate or severe rheumatoid arthritis cases, cyclosporine.

In one embodiment, the binding protein or antigen-binding portion thereof, is administered in combination with one of the following agents for the treatment of rheumatoid arthritis: a small molecule inhibitor of KDR, a small molecule inhibitor of Tie-2; methotrexate; prednisone; celecoxib; folic acid; hydroxychloroquine sulfate; rofecoxib; etanercept; infliximab; leflunomide; naproxen; valdecoxib; sulfasalazine; methylprednisolone; ibuprofen; meloxicam; methylprednisolone acetate; gold sodium thiomalate; aspirin; azathioprine; triamcinolone acetonide; propxyphene napsylate/apap; folate; nabumetone; diclofenac; piroxicam; etodolac; diclofenac sodium; oxaprozin; oxycodone hcl; hydrocodone bitartrate/apap; diclofenac sodium/misoprostol; fentanyl; anakinra, human recombinant; tramadol hcl; salsalate; sulindac; cyanocobalamin/fa/pyridoxine; acetaminophen; alendronate sodium; prednisolone; morphine sulfate; lidocaine hydrochloride; indomethacin; glucosamine sulfate/chondroitin; cyclosporine; amitriptyline hcl; sulfadiazine; oxycodone hcl/acetaminophen; olopatadine hcl; misoprostol; naproxen sodium; omeprazole; mycophenolate mofetil; cyclophosphamide; rituximab; IL-1 TRAP; MRA; CTLA4-IG; IL-18 BP; IL-12/23; anti-IL 18; anti-IL 15; BIRB-796; SC10-469; VX-702; AMG-548; VX-740; Roflumilast; IC-485; CDC-801; or mesopram.

Non-limiting examples of therapeutic agents for inflammatory bowel disease with which a binding protein provided herein can be combined include the following: budenoside; epidermal growth factor; a corticosteroid; cyclosporin, sulfasalazine; aminosalicylates; 6-mercaptopurine; azathioprine; metronidazole; a lipoxygenase inhibitor; mesalamine; olsalazine; balsalazide; an antioxidant; a thromboxane inhibitor; an IL-1 receptor antagonist; an anti-IL-1β mAb; an anti-IL-6 mAb; a growth factor; an elastase inhibitor; a pyridinyl-imidazole compound; an antibody to or antagonist of other human cytokines or growth factors, for example, TNF, LT, IL-1, IL-2, IL-6, IL-7, IL-8, IL-15, IL-16, IL-17, IL-18, EMAP-II, GM-CSF, FGF, or PDGF. Antibodies provided herein, or antigen binding portions thereof, can be combined with an antibody to a cell surface molecule such as CD2, CD3, CD4, CD8, CD25, CD28, CD30, CD40, CD45, CD69, CD90 or their ligands. The antibodies provided herein, or antigen binding portions thereof, may also be combined with an agent, such as methotrexate, cyclosporin, FK506, rapamycin, mycophenolate mofetil, leflunomide, an NSAID, for example, ibuprofen, a corticosteroid such as prednisolone, a phosphodiesterase inhibitor, an adenosine agonist, an antithrombotic agent, a complement inhibitor, an adrenergic agent, an agent which interferes with signalling by proinflammatory cytokines such as TNFα or IL-1 (e.g., an IRAK, NIK, IKK, p38 or MAP kinase inhibitor), an IL-1β converting enzyme inhibitor, a TNFα converting enzyme inhibitor, a T-cell signalling inhibitor such as a kinase inhibitor, a metalloproteinase inhibitor, sulfasalazine, azathioprine, a 6-mercaptopurine, an angiotensin converting enzyme inhibitor, a soluble cytokine receptor or derivative thereof (e.g., a soluble p55 or p75 TNF receptor, sIL-1RI, sIL-1RII, sIL-6R) or an antiinflammatory cytokine (e.g., IL-4, IL-10, IL-11, IL-13 or TGFβ) or a bcl-2 inhibitor.

Examples of therapeutic agents for Crohn's disease in which a binding protein can be combined include the following: a TNF antagonist, for example, an anti-TNF antibody, Adalimumab (PCT Publication No. WO 97/29131; HUMIRA), CA2 (REMICADE), CDP 571, a TNFR-Ig construct, (p75TNFRIgG (ENBREL) or a p55TNFRIgG (LENERCEPT)) inhibitor or a PDE4 inhibitor. Antibodies provided herein, or antigen binding portions thereof, can be combined with a corticosteroid, for example, budenoside and dexamethasone. Binding proteins provided herein or antigen binding portions thereof, may also be combined with an agent such as sulfasalazine, 5-aminosalicylic acid and olsalazine, or an agent that interferes with the synthesis or action of a proinflammatory cytokine such as IL-1, for example, an IL-1β converting enzyme inhibitor or IL-1ra. Antibodies provided herein or antigen binding portion thereof may also be used with a T cell signaling inhibitor, for example, a tyrosine kinase inhibitor or an 6-mercaptopurine. Binding proteins provided herein, or antigen binding portions thereof, can be combined with IL-11. Binding proteins provided herein, or antigen binding portions thereof, can be combined with mesalamine, prednisone, azathioprine, mercaptopurine, infliximab, methylprednisolone sodium succinate, diphenoxylate/atrop sulfate, loperamide hydrochloride, methotrexate, omeprazole, folate, ciprofloxacin/dextrose-water, hydrocodone bitartrate/apap, tetracycline hydrochloride, fluocinonide, metronidazole, thimerosal/boric acid, cholestyramine/sucrose, ciprofloxacin hydrochloride, hyoscyamine sulfate, meperidine hydrochloride, midazolam hydrochloride, oxycodone hcl/acetaminophen, promethazine hydrochloride, sodium phosphate, sulfamethoxazole/trimethoprim, celecoxib, polycarbophil, propoxyphene napsylate, hydrocortisone, multivitamins, balsalazide disodium, codeine phosphate/apap, colesevelam hcl, cyanocobalamin, folic acid, levofloxacin, methylprednisolone, natalizumab or interferon-gamma

Non-limiting examples of therapeutic agents for multiple sclerosis with which binding proteins provided herein can be combined include the following: a corticosteroid; prednisolone; methylprednisolone; azathioprine; cyclophosphamide; cyclosporine; methotrexate; 4-aminopyridine; tizanidine; interferon-β1a (AVONEX; Biogen); interferon-β1b (BETASERON; Chiron/Berlex); interferon α-n3) (Interferon Sciences/Fujimoto), interferon-α (Alfa Wassermann/J&J), interferon β1A-IF (Serono/Inhale Therapeutics), Peginterferon α 2b (Enzon/Schering-Plough), Copolymer 1 (Cop-1; COPAXONE; Teva Pharmaceutical Industries, Inc.); hyperbaric oxygen; intravenous immunoglobulin; clabribine; an antibody to or antagonist of other human cytokines or growth factors and their receptors, for example, TNF, LT, IL-1, IL-2, IL-6, IL-7, IL-8, IL-23, IL-15, IL-16, IL-18, EMAP-II, GM-CSF, FGF, or PDGF. Binding proteins provided herein can be combined with an antibody to a cell surface molecule such as CD2, CD3, CD4, CD8, CD19, CD20, CD25, CD28, CD30, CD40, CD45, CD69, CD80, CD86, CD90 or their ligands. Binding proteins provided herein, may also be combined with an agent, such as methotrexate, cyclosporine, FK506, rapamycin, mycophenolate mofetil, leflunomide, an NSAID, for example, ibuprofen, a corticosteroid such as prednisolone, a phosphodiesterase inhibitor, an adensosine agonist, an antithrombotic agent, a complement inhibitor, an adrenergic agent, an agent which interferes with signalling by a proinflammatory cytokine such as TNFα or IL-1 (e.g., IRAK, NIK, IKK, p38 or a MAP kinase inhibitor), an IL-1β converting enzyme inhibitor, a TACE inhibitor, a T-cell signaling inhibitor such as a kinase inhibitor, a metalloproteinase inhibitor, sulfasalazine, azathioprine, a 6-mercaptopurine, an angiotensin converting enzyme inhibitor, a soluble cytokine receptor or derivatives thereof (e.g., a soluble p55 or p75 TNF receptor, sIL-1RI, sIL-1RII, sIL-6R), an antiinflammatory cytokine (e.g., IL-4, IL-10, IL-13 or TGFβ) or a bcl-2 inhibitor.

Examples of therapeutic agents for multiple sclerosis in which binding proteins provided herein can be combined include interferon-β, for example, IFNβ1a and IFNβ1b; copaxone, corticosteroids, caspase inhibitors, for example inhibitors of caspase-1, IL-1 inhibitors, TNF inhibitors, and antibodies to CD40 ligand and CD80.

Non-limiting examples of therapeutic agents for asthma with which binding proteins provided herein can be combined include the following: albuterol, salmeterol/fluticasone, montelukast sodium, fluticasone propionate, budesonide, prednisone, salmeterol xinafoate, levalbuterol hcl, albuterol sulfate/ipratropium, prednisolone sodium phosphate, triamcinolone acetonide, beclomethasone dipropionate, ipratropium bromide, azithromycin, pirbuterol acetate, prednisolone, theophylline anhydrous, methylprednisolone sodium succinate, clarithromycin, zafirlukast, formoterol fumarate, influenza virus vaccine, methylprednisolone, amoxicillin trihydrate, flunisolide, allergy injection, cromolyn sodium, fexofenadine hydrochloride, flunisolide/menthol, amoxicillin/clavulanate, levofloxacin, inhaler assist device, guaifenesin, dexamethasone sodium phosphate, moxifloxacin hcl, doxycycline hyclate, guaifenesin/d-methorphan, p-ephedrine/cod/chlorphenir, gatifloxacin, cetirizine hydrochloride, mometasone furoate, salmeterol xinafoate, benzonatate, cephalexin, pe/hydrocodone/chlorphenir, cetirizine hcl/pseudoephed, phenylephrine/cod/promethazine, codeine/promethazine, cefprozil, dexamethasone, guaifenesin/pseudoephedrine, chlorpheniramine/hydrocodone, nedocromil sodium, terbutaline sulfate, epinephrine, methylprednisolone, metaproterenol sulfate.

Non-limiting examples of therapeutic agents for COPD with which binding proteins provided herein can be combined include the following: albuterol sulfate/ipratropium, ipratropium bromide, salmeterol/fluticasone, albuterol, salmeterol xinafoate, fluticasone propionate, prednisone, theophylline anhydrous, methylprednisolone sodium succinate, montelukast sodium, budesonide, formoterol fumarate, triamcinolone acetonide, levofloxacin, guaifenesin, azithromycin, beclomethasone dipropionate, levalbuterol hcl, flunisolide, ceftriaxone sodium, amoxicillin trihydrate, gatifloxacin, zafirlukast, amoxicillin/clavulanate, flunisolide/menthol, chlorpheniramine/hydrocodone, metaproterenol sulfate, methylprednisolone, mometasone furoate, p-ephedrine/cod/chlorphenir, pirbuterol acetate, p-ephedrine/loratadine, terbutaline sulfate, tiotropium bromide, (R,R)-formoterol, TgAAT, Cilomilast, Roflumilast.

Non-limiting examples of therapeutic agents for psoriasis with which binding proteins provided herein can be combined include the following: small molecule inhibitor of KDR, small molecule inhibitor of Tie-2, calcipotriene, clobetasol propionate, triamcinolone acetonide, halobetasol propionate, tazarotene, methotrexate, fluocinonide, betamethasone diprop augmented, fluocinolone acetonide, acitretin, tar shampoo, betamethasone valerate, mometasone furoate, ketoconazole, pramoxine/fluocinolone, hydrocortisone valerate, flurandrenolide, urea, betamethasone, clobetasol propionate/emoll, fluticasone propionate, azithromycin, hydrocortisone, moisturizing formula, folic acid, desonide, pimecrolimus, coal tar, diflorasone diacetate, etanercept folate, lactic acid, methoxsalen, hc/bismuth subgal/znox/resor, methylprednisolone acetate, prednisone, sunscreen, halcinonide, salicylic acid, anthralin, clocortolone pivalate, coal extract, coal tar/salicylic acid, coal tar/salicylic acid/sulfur, desoximetasone, diazepam, emollient, fluocinonide/emollient, mineral oil/castor oil/na lact, mineral oil/peanut oil, petroleum/isopropyl myristate, psoralen, salicylic acid, soap/tribromsalan, thimerosal/boric acid, celecoxib, infliximab, cyclosporine, alefacept, efalizumab, tacrolimus, pimecrolimus, PUVA, UVB, sulfasalazine.

Examples of therapeutic agents for SLE (Lupus) in which binding proteins provided herein can be combined include the following: NSAIDS, for example, diclofenac, naproxen, ibuprofen, piroxicam, indomethacin; COX2 inhibitors, for example, Celecoxib, rofecoxib, valdecoxib; anti-malarials, for example, hydroxychloroquine; Steroids, for example, prednisone, prednisolone, budenoside, dexamethasone; Cytotoxics, for example, azathioprine, cyclophosphamide, mycophenolate mofetil, methotrexate; inhibitors of PDE4 or purine synthesis inhibitor, for example Cellcept. Binding proteins provided herein may also be combined with agents such as sulfasalazine, 5-aminosalicylic acid, olsalazine, Imuran and agents which interfere with synthesis, production or action of proinflammatory cytokines such as IL-1, for example, caspase inhibitors like IL-1β converting enzyme inhibitors and IL-1ra. Binding proteins provided herein may also be used with T cell signaling inhibitors, for example, tyrosine kinase inhibitors; or molecules that target T cell activation molecules, for example, CTLA-4-IgG or anti-B7 family antibodies, anti-PD-1 family antibodies. Binding proteins provided herein, can be combined with IL-11 or anti-cytokine antibodies, for example, fonotolizumab (anti-IFNgamma antibody), or anti-receptor receptor antibodies, for example, anti-IL-6 receptor antibody and antibodies to B-cell surface molecules. Antibodies provided herein or antigen binding portion thereof may also be used with LJP 394 (abetimus), agents that deplete or inactivate B-cells, for example, Rituximab (anti-CD20 antibody), lymphostat-B (anti-BlyS antibody), TNF antagonists, for example, anti-TNF antibodies, Adalimumab (PCT Publication No. WO 97/29131; HUMIRA), CA2 (REMICADE), CDP 571, TNFR-Ig constructs, (p75TNFRIgG (ENBREL) and p55TNFRIgG (LENERCEPT)) and bcl-2 inhibitors, because bcl-2 overexpression in transgenic mice has been demonstrated to cause a lupus like phenotype (see Marquina The pharmaceutical compositions provided herein may include a “therapeutically effective amount” or a “prophylactically effective amount” of a binding protein provided herein. A “therapeutically effective amount” refers to an amount effective, at dosages and for periods of time necessary, to achieve the desired therapeutic result. A therapeutically effective amount of the binding protein may be determined by a person skilled in the art and may vary according to factors such as the disease state, age, sex, and weight of the individual, and the ability of the binding protein to elicit a desired response in the individual. A therapeutically effective amount is also one in which any toxic or detrimental effects of the antibody, or antibody binding portion, are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

### IV. Diagnostics

The disclosure herein also provides diagnostic applications including, but not limited to, diagnostic assay methods, diagnostic kits containing one or more binding proteins, and adaptation of the methods and kits for use in automated and/or semi-automated systems. The methods, kits, and adaptations provided may be employed in the detection, monitoring, and/or treatment of a disease or disorder in an individual. This is further elucidated below.

A. Method of Assay

The present disclosure also provides a method for determining the presence, amount or concentration of an analyte, or fragment thereof, in a test sample using at least one binding protein as described herein. Any suitable assay as is known in the art can be used in the method. Examples include, but are not limited to, immunoassays and/or methods employing mass spectrometry.

Immunoassays provided by the present disclosure may include sandwich immunoassays, radioimmunoassay (RIA), enzyme immunoassay (EIA), enzyme-linked immunosorbent assay (ELISA), competitive-inhibition immunoassays, fluorescence polarization immunoassay (FPIA), enzyme multiplied immunoassay technique (EMIT), bioluminescence resonance energy transfer (BRET), and homogenous chemiluminescent assays, among others.

A chemiluminescent microparticle immunoassay, in particular one employing the ARCHITECT® automated analyzer (Abbott Laboratories, Abbott Park, Ill.), is an example of an immunoassay.

Methods employing mass spectrometry are provided by the present disclosure and include, but are not limited to MALDI (matrix-assisted laser desorption/ionization) or by SELDI (surface-enhanced laser desorption/ionization).

Methods for collecting, handling, processing, and analyzing biological test samples using immunoassays and mass spectrometry would be well-known to one skilled in the art, are provided for in the practice of the present disclosure (US 2009-0311253 A1).

B. Kit

A kit for assaying a test sample for the presence, amount or concentration of an analyte, or fragment thereof, in a test sample is also provided. The kit comprises at least one component for assaying the test sample for the analyte, or fragment thereof, and instructions for assaying the test sample for the analyte, or fragment thereof. The at least one component for assaying the test sample for the analyte, or fragment thereof, can include a composition comprising a binding protein, as disclosed herein, and/or an anti-analyte binding protein (or a fragment, a variant, or a fragment of a variant thereof), which is optionally immobilized on a solid phase.

Optionally, the kit may comprise a calibrator or control, which may comprise isolated or purified analyte. The kit can comprise at least one component for assaying the test sample for an analyte by immunoassay and/or mass spectrometry. The kit components, including the analyte, binding protein, and/or anti-analyte binding protein, or fragments thereof, may be optionally labeled using any art-known detectable label. The materials and methods for the creation provided for in the practice of the present disclosure would be known to one skilled in the art (US 2009-0311253 A1).

C. Adaptation of Kit and Method

The kit (or components thereof), as well as the method of determining the presence, amount or concentration of an analyte in a test sample by an assay, such as an immunoassay as described herein, can be adapted for use in a variety of automated and semi-automated systems (including those wherein the solid phase comprises a microparticle), as described, for example, in U.S. Pat. Nos. 5,089,424 and 5,006,309, and as commercially marketed, for example, by Abbott Laboratories (Abbott Park, Ill.) as ARCHITECT®.

Other platforms available from Abbott Laboratories include, but are not limited to, AxSYM®, IMx® (see, for example, U.S. Pat. No. 5,294,404, PRISM®, EIA (bead), and Quantum™ II, as well as other platforms. Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories) electrochemical immunoassay system that performs sandwich immunoassays. Immunosensors and their methods of manufacture and operation in single-use test devices are described, for example in, U.S. Pat. Nos. 5,063,081, 7,419,821, and 7,682,833; and US Publication Nos. 20040018577, 20060160164 and US 20090311253.

It will be readily apparent to those skilled in the art that other suitable modifications and adaptations of the methods described herein are obvious and may be made using suitable equivalents without departing from the scope of the embodiments disclosed herein. Having now described certain embodiments in detail, the same will be more clearly understood by reference to the following examples, which are included for purposes of illustration only and are not intended to be limiting.

### EXAMPLES

### Example 1

### Design and Construction of Cross-Over DVD-Ig-Format 2

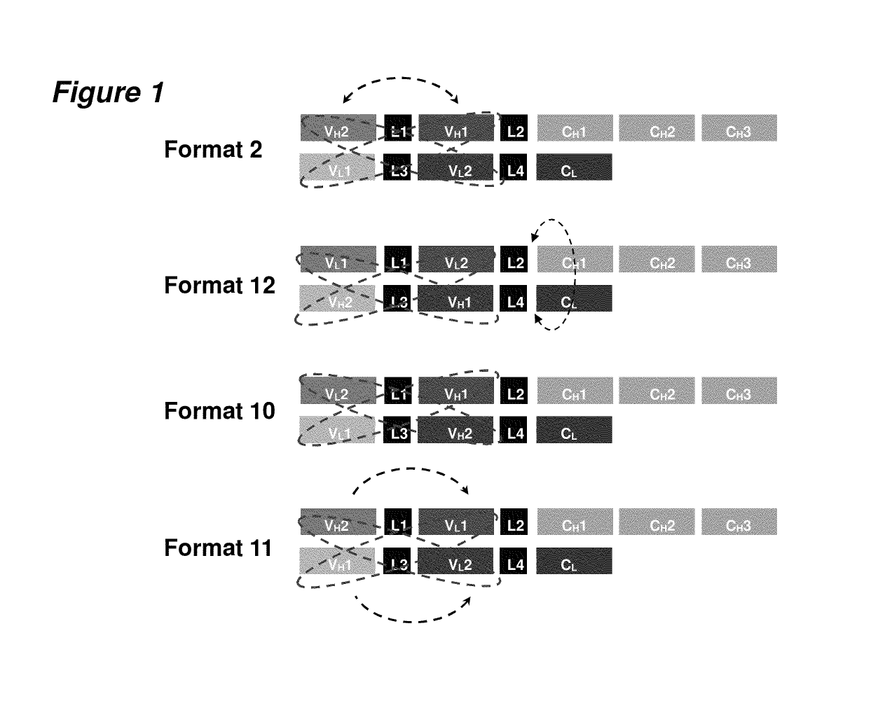

FIG. 1 shows one conformation model of format 2 DVD-Ig molecule. Because of the manner by which the variable domains are linked in this format, the inner binding domain may be fully exposed. In order to assess the effects of different lengths of the linker and to ensure that VH1 can form functional binding domain with VL1 while VH2 can form functional binding domain with VL2, various linker lengths are tested. A 7 amino acids long linker is used between VL2 and VL1, A 5 amino acids long linker is used between VL1 and CL, A 1 amino acid long linker is used between VH1 and VH2, and a 2 amino acids long linker is used between VH2 and CH1. See e.g., U.S. Pat. Application US20120251541 A1.

Variable domains in DVD1286 and DVD1282 are used to test cross-over DVD-Ig format 2. The sequences of the various clones are listed in Table 1. The sequence name ending with “.vh” is placed N-terminal to CH1-Fc (or CH-(X4)n). The sequence name ending with “.vl” is placed N-terminal to Ck (or CL-(X2)n). Sequences from the same clone are paired to form one cross-over DVD-Ig.

TABLE 1
Sequences of Format 2 variable domains

| | Sequence | |
|---|---|---|
| Clone | name | Sequence |
| 1 | B6- | EIVLTQSPDFQSVTPKEKVTITCRASQNIGSELHWYQQKPDQSPKLLIK |
| | 5G_1B12- | YASHSISGVPSRFSGSGSGTDFTLTINGLEAEDAATYYCHQSDTLPHTF |
| | 1.v1 | GQGTKVDIKGGGGGGGDTQVTQSPSSLSASVGDRVTITCITSTDIDVDM |
| | | NWYQQKPGKPPKLLISQGNTLRPGVPSRFSSSGSGTDFTFTISSLQPED |
| | | FATYYCLQSDNLPLTFGQGTKLEIKGGGGGR (SEQ ID No. 1) |
| 1 | 1B12-1_B6- | EVQLQESGPGLVKPSETLSLTCTVSGFSLSDYGVSWIRQPPGKGLEWLG |
| | 5G.vh | LIWGGGDTYYNSPLKSRLTISKDNSKSQVSLKLSSVTAADTAVYYCAKQ |
| | | RTLWGYDLYGMDYWGQGTLVTVSSGEVQLVQSGAEVKKPGESVKISCKA |
| | | SGGSFRSYGISWVRQAPGQGLEWMGGITHFFGITDYAQKFQGRVTITAD |
| | | ESTTTAYMELSGLTSDDTAVYYCAREPNDFWGGYYDTHDFDSWGQGTTV |
| | | TVSSGG (SEQ ID No. 2) |
| 2 | 1B12-1_B6- | DTQVTQSPSSLSASVGDRVTITCITSTDIDVDMNWYQQKPGKPPKLLIS |
| | 5G.v1 | QGNTLRPGVPSRFSSSGSGTDFTFTISSLQPEDFATYYCLQSDNLPLTF |
| | | GQGTKLEIKGGGGGGGEIVLTQSPDFQSVTPKEKVTITCRASQNIGSEL |
| | | HWYQQKPDQSPKLLIKYASHSISGVPSRFSGSGSGTDFTLTINGLEAED |
| | | AATYYCHQSDTLPHTFGQGTKVDIKGGGGGR (SEQ ID No. 3) |
| 2 | B6- | EVQLVQSGAEVKKPGESVKISCKASGGSFRSYGISWVRQAPGQGLEWMG |
| | 5G_1B12- | GITHFFGITDYAQKFQGRVTITADESTTTAYMELSGLTSDDTAVYYCAR |
| | 1.vh | EPNDFWGGYYDTHDFDSWGQGTTVTVSSGEVQLQESGPGLVKPSETLSL |
| | | TCTVSGFSLSDYGVSWIRQPPGKGLEWLGLIWGGGDTYYNSPLKSRLTI |
| | | SKDNSKSQVSLKLSSVTAADTAVYYCAKQRTLWGYDLYGMDYWGQGTLV |
| | | TVSSGG (SEQ ID No. 4) |
| 3 | B6-5G_E26- | EIVLTQSPDFQSVTPKEKVTITCRASQNIGSELHWYQQKPDQSPKLLIK |
| | 13.v1 | YASHSISGVPSRFSGSGSGTDFTLTINGLEAEDAATYYCHQSDTLPHTF |
| | | GQGTKVDIKGGGGGGGDIQMTQSPSSLSASVGDRVTITCRASGNIHNYL |
| | | TWYQQTPGKAPKLLIYNAKTLADGVPSRFSGSGSGTDYTFTISSLQPED |
| | | IATYYCQHFWSIPYTFGQGTKLQITGGGGGR (SEQ ID No. 5) |
| 3 | E26-13_B6- | EVQLVESGGGVVQPGRSLRLSCSASGFIFSRYDMSWVRQAPGKGLEWVA |
| | 5G.vh | YISHGGAGTYYPDSVKGRFTISRDNSKNTLFLQMDSLRPEDTGVYFCAR |
| | | GGVTKGYFDVWGQGTPVTVSSGEVQLVQSGAEVKKPGESVKISCKASGG |
| | | SFRSYGISWVRQAPGQGLEWMGGITHFFGITDYAQKFQGRVTITADEST |
| | | TTAYMELSGLTSDDTAVYYCAREPNDFWGGYYDTHDFDSWGQGTTVTVS |
| | | SGG (SEQ ID No. 6) |
| 4 | E26-13_B6- | DIQMTQSPSSLSASVGDRVTITCRASGNIHNYLTWYQQTPGKAPKLLIY |
| | 5G.v1 | NAKTLADGVPSRFSGSGSGTDYTFTISSLQPEDIATYYCQHFWSIPYTF |
| | | GQGTKLQITGGGGGGGEIVLTQSPDFQSVTPKEKVTITCRASQNIGSEL |
| | | HWYQQKPDQSPKLLIKYASHSISGVPSRFSGSGSGTDFTLTINGLEAED |
| | | AATYYCHQSDTLPHTFGQGTKVDIKGGGGGR (SEQ ID No. 7) |
| 4 | B6-5G_E26- | EVQLVQSGAEVKKPGESVKISCKASGGSFRSYGISWVRQAPGQGLEWMG |
| | 13.vh | GITHFFGITDYAQKFQGRVTITADESTTTAYMELSGLTSDDTAVYYCAR |
| | | EPNDFWGGYYDTHDFDSWGQGTTVTVSSGEVQLVESGGGVVQPGRSLRL |
| | | SCSASGFIFSRYDMSWVRQAPGKGLEWVAYISHGGAGTYYPDSVKGRFT |
| | | ISRDNSKNTLFLQMDSLRPEDTGVYFCARGGVTKGYEDVWGQGTPVTVS |
| | | SGG (SEQ ID No. 8) |
| 17 | B6- | EIVLTQSPDFQSVTPKEKVTITCRASQNIGSELHWYQQKPDQSPKLLIK |
| | 5G_1B12- | YASHSISGVPSRFSGSGSGTDFTLTINGLEAEDAATYYCHQSDTLPHTF |
| | A3.v1 | GQGTKVDIKGGGSGGGDIQMTQSPSSLSASVGDRVTITCQASTDIDDDL |
| | | NWYQQKPGKAPKLLISLGSTLRPGVPSRFSGSGSGTDFTFTISSLQPED |
| | | FATYYCLQSDRLPLTFGQGTKLEIKGGGSGR (SEQ ID No. X) |
| 17 | 1B12- | EVQLQESGPGLVKPSETLSLTCTVSGFSLSDYGVSWIRQPPGKGLEWLG |
| | A3_B6- | LIWGGGDTYYNSPLKSRLTISKDNSKSQVSLKLSSVTAADTAVYYCARQ |
| | 5G.vh | TNLWAYDLYSMDYWGQGTLVTVSSGEVQLVQSGAEVKKPGESVKISCKA |
| | | SGGSFRSYGISWVRQAPGQGLEWMGGITHFFGITDYAQKFQGRVTITAD |
| | | ESTTTAYMELSGLTSDDTAVYYCAREPNDFWGGYYDTHDFDSWGQGTTV |
| | | TVSSGG (SEQ ID No. X) |
| 18 | 1B12- | DIQMTQSPSSLSASVGDRVTITCQASTDIDDDLNWYQQKPGKAPKLLIS |
| | A3_B6- | LGSTLRPGVPSRFSGSGSGTDFTFTISSLQPEDFATYYCLQSDRLPLTF |
| | 5G.v1 | GQGTKLEIKGGGSGGGEIVLTQSPDFQSVTPKEKVTITCRASQNIGSEL |
| | | HWYQQKPDQSPKLLIKYASHSISGVPSRFSGSGSGTDFTLTINGLEAED |
| | | AATYYCHQSDTLPHTFGQGTKVDIKGGGSGR (SEQ ID No. X) |
| 18 | B6- | EVQLVQSGAEVKKPGESVKISCKASGGSFRSYGISWVRQAPGQGLEWMG |
| | 5G_1B12- | GITHFFGITDYAQKFQGRVTITADESTTTAYMELSGLTSDDTAVYYCAR |
| | A3.vh | EPNDFWGGYYDTHDFDSWGQGTTVTVSSGEVQLQESGPGLVKPSETLSL |
| | | TCTVSGFSLSDYGVSWIRQPPGKGLEWLGLIWGGGDTYYNSPLKSRLTI |
| | | SKDNSKSQVSLKLSSVTAADTAVYYCARQTNLWAYDLYSMDYWGQGTLV |
| | | TVSSGG (SEQ ID No. X) |

### Example 2

### Design and Construction of Cross-Over DVD-Ig-Format 10

FIG. 1 shows one conformation model of format 10 DVD-Ig molecule. Because of the manner by which the variable domains are linked in this format, the inner binding domain may be fully exposed. In order to assess the effects of different lengths of the linker and to ensure that VH1 can form functional binding domain with VL1 while VH2 can form functional binding domain with VL2, various linker lengths are tested. A linker having the sequence of GGGSGGGG is used between VL2 and VH1, a linker having the sequence of LGGCGGGS is used between VH1 and CH1, a linker having the sequence of GGGSGGGG is used between VL1 and VH2, a linker having the sequence of LGGCGGGS is used between VH2 and CL. See e.g., U.S. Pat. Application 20090060910A1. Various changes in linker length and sequences may be made to optimize the choice.

Variable domains in DVD1286 and DVD1282 are used to test cross-over DVD-Ig format 10. The sequences of the various clones are listed in Table 2. The sequence name ending with “.vh” is placed N-terminal to CH1-Fc (or CH-(X4)n). The sequence name ending with “.vl” is placed N-terminal to Ck (or CL-(X2)n). Sequences from the same clone are paired to form one cross-over DVD-Ig.

TABLE 2
Sequences of Format 10 variable domains

| | Sequence | |
|---|---|---|
| Clone | name | Sequence |
| 5 | 1B12-1_B6- | DTQVTQSPSSLSASVGDRVTITCITSTDIDVDMNWYQQKPGKPPKLLIS |
| | 5G.v1 | QGNTLRPGVPSRFSSSGSGTDFTFTISSLQPEDFATYYCLQSDNLPLTF |
| | | GQGTKLEIKGGGSGGGGEVQLVQSGAEVKKPGESVKISCKASGGSFRSY |
| | | GISWVRQAPGQGLEWMGGITHFFGITDYAQKFQGRVTITADESTTTAYM |
| | | ELSGLTSDDTAVYYCAREPNDFWGGYYDTHDFDSWGQGTTVTVSSLGGC |
| | | GGGSR (SEQ ID No. 9) |
| 5 | B6- | EIVLTQSPDFQSVTPKEKVTITCRASQNIGSELHWYQQKPDQSPKLLIK |
| | 5G_1B12- | YASHSISGVPSRFSGSGSGTDFTLTINGLEAEDAATYYCHQSDTLPHTF |
| | 1.vh | GQGTKVDIKGGGSGGGGEVQLQESGPGLVKPSETLSLTCTVSGFSLSDY |
| | | GVSWIRQPPGKGLEWLGLIWGGGDTYYNSPLKSRLTISKDNSKSQVSLK |
| | | LSSVTAADTAVYYCAKQRTLWGYDLYGMDYWGQGTLVTVSSLGGCGGGS |
| | | (SEQ ID No. 10) |
| 6 | B6- | EIVLTQSPDFQSVTPKEKVTITCRASQNIGSELHWYQQKPDQSPKLLIK |
| | 5G_1B12- | YASHSISGVPSRFSGSGSGTDFTLTINGLEAEDAATYYCHQSDTLPHTF |
| | 1.v1 | GQGTKVDIKGGGSGGGGEVQLQESGPGLVKPSETLSLTCTVSGFSLSDY |
| | | GVSWIRQPPGKGLEWLGLIWGGGDTYYNSPLKSRLTISKDNSKSQVSLK |
| | | LSSVTAADTAVYYCAKQRTLWGYDLYGMDYWGQGTLVTVSSLGGCGGGS |
| | | R (SEQ ID No. 11) |
| 6 | 1B12-1_B6- | DTQVTQSPSSLSASVGDRVTITCITSTDIDVDMNWYQQKPGKPPKLLIS |
| | 5G.vh | QGNTLRPGVPSRFSSSGSGTDFTFTISSLQPEDFATYYCLQSDNLPLTF |
| | | GQGTKLEIKGGGSGGGGEVQLVQSGAEVKKPGESVKISCKASGGSFRSY |
| | | GISWVRQAPGQGLEWMGGITHFFGITDYAQKFQGRVTITADESTTTAYM |
| | | ELSGLTSDDTAVYYCAREPNDFWGGYYDTHDFDSWGQGTTVTVSSLGGC |
| | | GGGS (SEQ ID No. 12) |
| 7 | E26-13_B6- | DIQMTQSPSSLSASVGDRVTITCRASGNIHNYLTWYQQTPGKAPKLLIY |
| | 5G.v1 | NAKTLADGVPSRFSGSGSGTDYTFTISSLQPEDIATYYCQHFWSIPYTF |
| | | GQGTKLQITGGGSGGGGEVQLVQSGAEVKKPGESVKISCKASGGSFRSY |
| | | GISWVRQAPGQGLEWMGGITHFFGITDYAQKFQGRVTITADESTTTAYM |
| | | ELSGLTSDDTAVYYCAREPNDFWGGYYDTHDFDSWGQGTTVTVSSLGGC |
| | | GGGSR (SEQ ID No. 13) |
| 7 | B6-5G_E26- | EIVLTQSPDFQSVTPKEKVTITCRASQNIGSELHWYQQKPDQSPKLLIK |
| | 13.vh | YASHSISGVPSRFSGSGSGTDFTLTINGLEAEDAATYYCHQSDTLPHTF |
| | | GQGTKVDIKGGGSGGGGEVQLVESGGGVVQPGRSLRLSCSASGFIFSRY |
| | | DMSWVRQAPGKGLEWVAYISHGGAGTYYPDSVKGRFTISRDNSKNTLFL |
| | | QMDSLRPEDTGVYFCARGGVTKGYFDVWGQGTPVTVSSLGGCGGGS |
| | | (SEQ ID No. 14) |
| 8 | B6-5G_E26- | EIVLTQSPDFQSVTPKEKVTITCRASQNIGSELHWYQQKPDQSPKLLIK |
| | 13.v1 | YASHSISGVPSRFSGSGSGTDFTLTINGLEAEDAATYYCHQSDTLPHTF |
| | | GQGTKVDIKGGGSGGGGEVQLVESGGGVVQPGRSLRLSCSASGFIFSRY |
| | | DMSWVRQAPGKGLEWVAYISHGGAGTYYPDSVKGRFTISRDNSKNTLFL |
| | | QMDSLRPEDTGVYFCARGGVTKGYFDVWGQGTPVTVSSLGGCGGGSR |
| | | (SEQ ID No. 15) |
| 8 | E26-13_B6- | DIQMTQSPSSLSASVGDRVTITCRASGNIHNYLTWYQQTPGKAPKLLIY |
| | 5G.vh | NAKTLADGVPSRFSGSGSGTDYTFTISSLQPEDIATYYCQHFWSIPYTF |
| | | GQGTKLQITGGGSGGGGEVQLVQSGAEVKKPGESVKISCKASGGSFRSY |
| | | GISWVRQAPGQGLEWMGGITHFFGITDYAQKFQGRVTITADESTTTAYM |
| | | ELSGLTSDDTAVYYCAREPNDFWGGYYDTHDFDSWGQGTTVTVSSLGGC |
| | | GGGS (SEQ ID No. 16) |
| 19 | 1B12- | DIQMTQSPSSLSASVGDRVTITCQASTDIDDDLNWYQQKPGKAPKLLIS |
| | A3_B6- | LGSTLRPGVPSRFSGSGSGTDFTFTISSLQPEDFATYYCLQSDRLPLTF |
| | 5G.v1 | GQGTKLEIKGGGSGGGGEVQLVQSGAEVKKPGESVKISCKASGGSFRSY |
| | | GISWVRQAPGQGLEWMGGITHFFGITDYAQKFQGRVTITADESTTTAYM |
| | | ELSGLTSDDTAVYYCAREPNDFWGGYYDTHDFDSWGQGTTVTVSSLGGC |
| | | GGGSR (SEQ ID No. X) |
| 19 | B6- | EIVLTQSPDFQSVTPKEKVTITCRASQNIGSELHWYQQKPDQSPKLLIK |
| | 5G_1B12- | YASHSISGVPSRFSGSGSGTDFTLTINGLEAEDAATYYCHQSDTLPHTF |
| | A3.vh | GQGTKVDIKGGGSGGGGEVQLQESGPGLVKPSETLSLTCTVSGFSLSDY |
| | | GVSWIRQPPGKGLEWLGLIWGGGDTYYNSPLKSRLTISKDNSKSQVSLK |
| | | LSSVTAADTAVYYCARQTNLWAYDLYSMDYWGQGTLVTVSSLGGCGGGS |
| | | (SEQ ID No. X) |
| 20 | B6- | EIVLTQSPDFQSVTPKEKVTITCRASQNIGSELHWYQQKPDQSPKLLIK |
| | 5G_1B12- | YASHSISGVPSRFSGSGSGTDFTLTINGLEAEDAATYYCHQSDTLPHTF |
| | A3.v1 | GQGTKVDIKGGGSGGGGEVQLQESGPGLVKPSETLSLTCTVSGFSLSDY |
| | | GVSWIRQPPGKGLEWLGLIWGGGDTYYNSPLKSRLTISKDNSKSQVSLK |
| | | LSSVTAADTAVYYCARQTNLWAYDLYSMDYWGQGTLVTVSSLGGCGGGS |
| | | R (SEQ ID No. X) |
| 20 | 1B12- | DIQMTQSPSSLSASVGDRVTITCQASTDIDDDLNWYQQKPGKAPKLLIS |
| | A3_B6- | LGSTLRPGVPSRFSGSGSGTDFTFTISSLQPEDFATYYCLQSDRLPLTF |
| | 5G.vh | GQGTKLEIKGGGSGGGGEVQLVQSGAEVKKPGESVKISCKASGGSFRSY |
| | | GISWVRQAPGQGLEWMGGITHFFGITDYAQKFQGRVTITADESTTTAYM |
| | | ELSGLTSDDTAVYYCAREPNDFWGGYYDTHDFDSWGQGTTVTVSSLGGC |
| | | GGGS (SEQ ID No. X) |

### Example 3

### Design and Construction of Cross-Over DVD-Ig-Format 11

FIG. 1 shows one conformation model of DVD-Ig molecule in format 11. Because of the manner by which the variable domains are linked in this format, the inner binding domain may be fully exposed. In order to assess the effects of different lengths of the linker and to ensure that VH1 can form functional binding domain with VL1 while VH2 can form functional binding domain with VL2, various linker lengths are tested. Based on modeled possible relative position between VH1 and VL1 and relative position between VH2 and VL2, linker between VH2 and VL1 is 3-12 amino acids in length, linker between VL1 and CH1 is 3-8 amino acids long, linker between VH1 and VL2 is 5-12 amino acids long, linker between VL2 and CL is 1-3 amino acids long. Various changes in linker length and sequences may be made to optimize the formation and the exposure of the two antigen binding domains.

Variable domains in DVD1286 and DVD1282 are used to test cross-over DVD-Ig format 11. The sequences of the various clones are listed in Table 3. The sequence name ending with “.vh” is placed N-terminal to CH1-Fc (or CH-(X4)n). The sequence name ending with “.vl” is placed N-terminal to Ck (or CL-(X2)n). Sequences from the same clone are paired to form one cross-over DVD-Ig.

TABLE 3
Sequences of Format 11 variable domains

| | Sequence | |
|---|---|---|
| Clone | name | Sequence |
| 9 | 1B12-1_B6- | EVQLQESGPGLVKPSETLSLTCTVSGFSLSDYGVSWIRQPPGKGLEWLG |
| | 5G.v1 | LIWGGGDTYYNSPLKSRLTISKDNSKSQVSLKLSSVTAADTAVYYCAKQ |
| | | RTLWGYDLYGMDYWGQGTLVTVSSGGGGSGGGEIVLTQSPDFQSVTPKE |
| | | KVTITCRASQNIGSELHWYQQKPDQSPKLLIKYASHSISGVPSRFSGSG |
| | | SGTDFTLTINGLEAEDAATYYCHQSDTLPHTFGQGTKVDIKGGGR |
| | | (SEQ ID No. 17) |
| 9 | B6- | EVQLVQSGAEVKKPGESVKISCKASGGSFRSYGISWVRQAPGQGLEWMG |
| | 5G_1B12.vh | GITHFFGITDYAQKFQGRVTITADESTTTAYMELSGLTSDDTAVYYCAR |
| | | EPNDFWGGYYDTHDFDSWGQGTTVTVSSGGGGSGGGDTQVTQSPSSLSA |
| | | SVGDRVTITCITSTDIDVDMNWYQQKPGKPPKLLISQGNTLRPGVPSRF |
| | | SSSGSGTDFTFTISSLQPEDFATYYCLQSDNLPLTFGQGTKLEIKGGGS |
| | | G (SEQ ID No. 18) |
| 10 | B6- | EVQLVQSGAEVKKPGESVKISCKASGGSFRSYGISWVRQAPGQGLEWMG |
| | 5G_1B12.v1 | GITHFFGITDYAQKFQGRVTITADESTTTAYMELSGLTSDDTAVYYCAR |
| | | EPNDFWGGYYDTHDFDSWGQGTTVTVSSGGGGSGGGDTQVTQSPSSLSA |
| | | SVGDRVTITCITSTDIDVDMNWYQQKPGKPPKLLISQGNTLRPGVPSRF |
| | | SSSGSGTDFTFTISSLQPEDFATYYCLQSDNLPLTFGQGTKLEIKGGGR |
| | | (SEQ ID No. 19) |
| 10 | 1B12-1_B6- | EVQLQESGPGLVKPSETLSLTCTVSGFSLSDYGVSWIRQPPGKGLEWLG |
| | 5G.vh | LIWGGGDTYYNSPLKSRLTISKDNSKSQVSLKLSSVTAADTAVYYCAKQ |
| | | RTLWGYDLYGMDYWGQGTLVTVSSGGGGSGGGEIVLTQSPDFQSVTPKE |
| | | KVTITCRASQNIGSELHWYQQKPDQSPKLLIKYASHSISGVPSRFSGSG |
| | | SGTDFTLTINGLEAEDAATYYCHQSDTLPHTFGQGTKVDIKGGGSG |
| | | (SEQ ID No. 20) |
| 11 | E26-13_B6- | EVQLVESGGGVVQPGRSLRLSCSASGFIFSRYDMSWVRQAPGKGLEWVA |
| | 5G.v1 | YISHGGAGTYYPDSVKGRFTISRDNSKNTLFLQMDSLRPEDTGVYFCAR |
| | | GGVTKGYFDVWGQGTPVTVSSGGGGSGGGEIVLTQSPDFQSVTPKEKVT |
| | | ITCRASQNIGSELHWYQQKPDQSPKLLIKYASHSISGVPSRFSGSGSGT |
| | | DFTLTINGLEAEDAATYYCHQSDTLPHTFGQGTKVDIKGGGR (SEQ |
| | | ID No. 21) |
| 11 | B6-5G_E26- | EVQLVQSGAEVKKPGESVKISCKASGGSFRSYGISWVRQAPGQGLEWMG |
| | 13.vh | GITHFFGITDYAQKFQGRVTITADESTTTAYMELSGLTSDDTAVYYCAR |
| | | EPNDFWGGYYDTHDFDSWGQGTTVTVSSGGGGSGGGDIQMTQSPSSLSA |
| | | SVGDRVTITCRASGNIHNYLTWYQQTPGKAPKLLIYNAKTLADGVPSRF |
| | | SGSGSGTDYTFTISSLQPEDIATYYCQHFWSIPYTFGQGTKLQITGGSG |
| | | G (SEQ ID No. 22) |
| 12 | B6-5G_E26- | EVQLVQSGAEVKKPGESVKISCKASGGSFRSYGISWVRQAPGQGLEWMG |
| | 13.v1 | GITHFFGITDYAQKFQGRVTITADESTTTAYMELSGLTSDDTAVYYCAR |
| | | EPNDFWGGYYDTHDFDSWGQGTTVTVSSGGGGSGGGDIQMTQSPSSLSA |
| | | SVGDRVTITCRASGNIHNYLTWYQQTPGKAPKLLIYNAKTLADGVPSRF |
| | | SGSGSGTDYTFTISSLQPEDIATYYCQHFWSIPYTFGQGTKLQITGGGR |
| | | (SEQ ID No. 23) |
| 12 | E26-13_B6- | EVQLVESGGGVVQPGRSLRLSCSASGFIFSRYDMSWVRQAPGKGLEWVA |
| | 5G.vh | YISHGGAGTYYPDSVKGRFTISRDNSKNTLFLQMDSLRPEDTGVYFCAR |
| | | GGVTKGYFDVWGQGTPVTVSSGGGGSGGGEIVLTQSPDFQSVTPKEKVT |
| | | ITCRASQNIGSELHWYQQKPDQSPKLLIKYASHSISGVPSRFSGSGSGT |
| | | DFTLTINGLEAEDAATYYCHQSDTLPHTFGQGTKVDIKGGSGG (SEQ |
| | | ID No. 24) |
| 21 | 1B12- | EVQLQESGPGLVKPSETLSLTCTVSGFSLSDYGVSWIRQPPGKGLEWLG |
| | A3_B6- | LIWGGGDTYYNSPLKSRLTISKDNSKSQVSLKLSSVTAADTAVYYCARQ |
| | 5G.v1 | TNLWAYDLYSMDYWGQGTLVTVSSGGGGSGGGEIVLTQSPDFQSVTPKE |
| | | KVTITCRASQNIGSELHWYQQKPDQSPKLLIKYASHSISGVPSRFSGSG |
| | | SGTDFTLTINGLEAEDAATYYCHQSDTLPHTFGQGTKVDIKGGGR |
| | | (SEQ ID No. X) |
| 21 | B6- | EVQLVQSGAEVKKPGESVKISCKASGGSFRSYGISWVRQAPGQGLEWMG |
| | 5G_1B12- | GITHFFGITDYAQKFQGRVTITADESTTTAYMELSGLTSDDTAVYYCAR |
| | A3.vh | EPNDFWGGYYDTHDFDSWGQGTTVTVSSGGGGSGGGDIQMTQSPSSLSA |
| | | SVGDRVTITCQASTDIDDDLNWYQQKPGKAPKLLISLGSTLRPGVPSRF |
| | | SGSGSGTDFTFTISSLQPEDFATYYCLQSDRLPLTFGQGTKLEIKGGGS |
| | | G (SEQ ID No. X) |
| 22 | B6- | EVQLVQSGAEVKKPGESVKISCKASGGSFRSYGISWVRQAPGQGLEWMG |
| | 5G_1B12- | GITHFFGITDYAQKFQGRVTITADESTTTAYMELSGLTSDDTAVYYCAR |
| | A3.v1 | EPNDFWGGYYDTHDFDSWGQGTTVTVSSGGGGSGGGDIQMTQSPSSLSA |
| | | SVGDRVTITCQASTDIDDDLNWYQQKPGKAPKLLISLGSTLRPGVPSRF |
| | | SGSGSGTDFTFTISSLQPEDFATYYCLQSDRLPLTFGQGTKLEIKGGGR |
| | | (SEQ ID No. X) |
| 22 | 1B12- | EVQLQESGPGLVKPSETLSLTCTVSGFSLSDYGVSWIRQPPGKGLEWLG |
| | A3_B6- | LIWGGGDTYYNSPLKSRLTISKDNSKSQVSLKLSSVTAADTAVYYCARQ |
| | 5G.vh | TNLWAYDLYSMDYWGQGTLVTVSSGGGGSGGGEIVLTQSPDFQSVTPKE |
| | | KVTITCRASQNIGSELHWYQQKPDQSPKLLIKYASHSISGVPSRFSGSG |
| | | SGTDFTLTINGLEAEDAATYYCHQSDTLPHTFGQGTKVDIKGGGSG |
| | | (SEQ ID No. X) |

### Example 4

### Design and Construction of Cross-Over DVD-Ig-Format 12

FIG. 1 shows one conformation model of DVD-Ig molecule in format 12 using 1D4.1-ss-ABT325 DVD-Ig as the structure template. Because of the manner by which the variable domains are linked in this format, the inner binding domain may be fully exposed. In order to assess the effects of different lengths of the linker and to ensure that VH1 can form functional binding domain with VL1 while VH2 can form functional binding domain with VL2, various linker lengths are tested. Based on modeled relative position between VH1 and VL1 and relative position between VH2 and VL2, the linker between VH1 and VH2 is 1-5 amino acids long, the linker between VH2 and CL is 1-3 amino acids long, the linker between VL2 and VL1 is 5-15 amino acids long, and the linker between VL1 and CH is 3-8 amino acids long. Various changes in linker length and linker sequences may be made to optimize the formation and the exposure of the two antigen binding sites.

Variable domains in DVD1286 and DVD1282 are used to test cross-over DVD-Ig format 12. The sequences of the various clones are listed in Table 4. The sequence name ending with “.vh” is placed N-terminal to CH1-Fc (or CH-(X4)n). The sequence name ending with “.vl” is placed N-terminal to Ck (or CL-(X2)n). Sequences from the same clone are paired to form one cross-over DVD-Ig.

TABLE 4
Sequences of Format 11 variable domains

| | Sequence | |
|---|---|---|
| Clone | name | Sequence |
| 13 | 1B12-1_B6- | EVQLQESGPGLVKPSETLSLTCTVSGFSLSDYGVSWIRQPPGKGLEWLG |
| | 5G.v1 | LIWGGGDTYYNSPLKSRLTISKDNSKSQVSLKLSSVTAADTAVYYCAKQ |
| | | RTLWGYDLYGMDYWGQGTLVTVSSGEVQLVQSGAEVKKPGESVKISCKA |
| | | SGGSFRSYGISWVRQAPGQGLEWMGGITHFFGITDYAQKFQGRVTITAD |
| | | ESTTTAYMELSGLTSDDTAVYYCAREPNDFWGGYYDTHDFDSWGQGTTV |
| | | TVSSGGR (SEQ ID No. 25) |
| 13 | B6- | EIVLTQSPDFQSVTPKEKVTITCRASQNIGSELHWYQQKPDQSPKLLIK |
| | 5G_1B12- | YASHSISGVPSRFSGSGSGTDFTLTINGLEAEDAATYYCHQSDTLPHTF |
| | 1.vh | GQGTKVDIKGGGGGGGDTQVTQSPSSLSASVGDRVTITCITSTDIDVDM |
| | | NWYQQKPGKPPKLLISQGNTLRPGVPSRFSSSGSGTDFTFTISSLQPED |
| | | FATYYCLQSDNLPLTFGQGTKLEIKGGGGG (SEQ ID No. 26) |
| 14 | B6- | EVQLVQSGAEVKKPGESVKISCKASGGSFRSYGISWVRQAPGQGLEWMG |
| | 5G_1B12- | GITHFFGITDYAQKFQGRVTITADESTTTAYMELSGLTSDDTAVYYCAR |
| | 1.v1 | EPNDFWGGYYDTHDFDSWGQGTTVTVSSGEVQLQESGPGLVKPSETLSL |
| | | TCTVSGFSLSDYGVSWIRQPPGKGLEWLGLIWGGGDTYYNSPLKSRLTI |
| | | SKDNSKSQVSLKLSSVTAADTAVYYCAKQRTLWGYDLYGMDYWGQGTLV |
| | | TVSSGGR (SEQ ID No. 27) |
| 14 | 1B12-1_B6- | DTQVTQSPSSLSASVGDRVTITCITSTDIDVDMNWYQQKPGKPPKLLIS |
| | 5G.vh | QGNTLRPGVPSRFSSSGSGTDFTFTISSLQPEDFATYYCLQSDNLPLTF |
| | | GQGTKLEIKGGGGGGGEIVLTQSPDFQSVTPKEKVTITCRASQNIGSEL |
| | | HWYQQKPDQSPKLLIKYASHSISGVPSRFSGSGSGTDFTLTINGLEAED |
| | | AATYYCHQSDTLPHTFGQGTKVDIKGGGGG (SEQ ID No. 28) |
| 15 | E26-13_B6- | EVQLVESGGGVVQPGRSLRLSCSASGFIFSRYDMSWVRQAPGKGLEWVA |
| | 5G.v1 | YISHGGAGTYYPDSVKGRFTISRDNSKNTLFLQMDSLRPEDTGVYFCAR |
| | | GGVTKGYFDVWGQGTPVTVSSGEVQLVQSGAEVKKPGESVKISCKASGG |
| | | SFRSYGISWVRQAPGQGLEWMGGITHFFGITDYAQKFQGRVTITADEST |
| | | TTAYMELSGLTSDDTAVYYCAREPNDFWGGYYDTHDFDSWGQGTTVTVS |
| | | SGGR (SEQ ID No. 29) |
| 15 | B6-5G_E26- | EIVLTQSPDFQSVTPKEKVTITCRASQNIGSELHWYQQKPDQSPKLLIK |
| | 13.vh | YASHSISGVPSRFSGSGSGTDFTLTINGLEAEDAATYYCHQSDTLPHTF |
| | | GQGTKVDIKGGGGGGGDIQMTQSPSSLSASVGDRVTITCRASGNIHNYL |
| | | TWYQQTPGKAPKLLIYNAKTLADGVPSRFSGSGSGTDYTFTISSLQPED |
| | | IATYYCQHFWSIPYTFGQGTKLQITGGGGG (SEQ ID No. 30) |
| 16 | B6-5G_E26- | EVQLVQSGAEVKKPGESVKISCKASGGSFRSYGISWVRQAPGQGLEWMG |
| | 13.v1 | GITHFFGITDYAQKFQGRVTITADESTTTAYMELSGLTSDDTAVYYCAR |
| | | EPNDFWGGYYDTHDFDSWGQGTTVTVSSGEVQLVESGGGVVQPGRSLRL |
| | | SCSASGFIFSRYDMSWVRQAPGKGLEWVAYISHGGAGTYYPDSVKGRFT |
| | | ISRDNSKNTLFLQMDSLRPEDTGVYFCARGGVTKGYEDVWGQGTPVTVS |
| | | SGGR (SEQ ID No. 31) |
| 16 | E26-13_B6- | DIQMTQSPSSLSASVGDRVTITCRASGNIHNYLTWYQQTPGKAPKLLIY |
| | 5G.vh | NAKTLADGVPSRFSGSGSGTDYTFTISSLQPEDIATYYCQHFWSIPYTF |
| | | GQGTKLQITGGGGGGGEIVLTQSPDFQSVTPKEKVTITCRASQNIGSEL |
| | | HWYQQKPDQSPKLLIKYASHSISGVPSRFSGSGSGTDFTLTINGLEAED |
| | | AATYYCHQSDTLPHTFGQGTKVDIKGGGGG (SEQ ID No. 32) |
| 23 | 1B12- | EVQLQESGPGLVKPSETLSLTCTVSGFSLSDYGVSWIRQPPGKGLEWLG |
| | A3_B6- | LIWGGGDTYYNSPLKSRLTISKDNSKSQVSLKLSSVTAADTAVYYCARQ |
| | 5G.v1 | TNLWAYDLYSMDYWGQGTLVTVSSGEVQLVQSGAEVKKPGESVKISCKA |
| | | SGGSFRSYGISWVRQAPGQGLEWMGGITHFFGITDYAQKFQGRVTITAD |
| | | ESTTTAYMELSGLTSDDTAVYYCAREPNDFWGGYYDTHDFDSWGQGTTV |
| | | TVSSGGR |
| 23 | B6- | EIVLTQSPDFQSVTPKEKVTITCRASQNIGSELHWYQQKPDQSPKLLIK |
| | 5G_1B12- | YASHSISGVPSRFSGSGSGTDFTLTINGLEAEDAATYYCHQSDTLPHTF |
| | A3.vh | GQGTKVDIKGGGSGGGDIQMTQSPSSLSASVGDRVTITCQASTDIDDDL |
| | | NWYQQKPGKAPKLLISLGSTLRPGVPSRFSGSGSGTDFTFTISSLQPED |
| | | FATYYCLQSDRLPLTFGQGTKLEIKGGGSG |
| 24 | B6- | EVQLVQSGAEVKKPGESVKISCKASGGSFRSYGISWVRQAPGQGLEWMG |
| | 5G_1B12- | GITHFFGITDYAQKFQGRVTITADESTTTAYMELSGLTSDDTAVYYCAR |
| | A3.v1 | EPNDFWGGYYDTHDFDSWGQGTTVTVSSGEVQLQESGPGLVKPSETLSL |
| | | TCTVSGFSLSDYGVSWIRQPPGKGLEWLGLIWGGGDTYYNSPLKSRLTI |
| | | SKDNSKSQVSLKLSSVTAADTAVYYCARQTNLWAYDLYSMDYWGQGTLV |
| | | TVSSGGR |
| 24 | 1B12- | DIQMTQSPSSLSASVGDRVTITCQASTDIDDDLNWYQQKPGKAPKLLIS |
| | A3_B6- | LGSTLRPGVPSRFSGSGSGTDFTFTISSLQPEDFATYYCLQSDRLPLTF |
| | 5G.vh | GQGTKLEIKGGGSGGGEIVLTQSPDFQSVTPKEKVTITCRASQNIGSEL |
| | | HWYQQKPDQSPKLLIKYASHSISGVPSRFSGSGSGTDFTLTINGLEAED |
| | | AATYYCHQSDTLPHTFGQGTKVDIKGGGSG |

### Example 5

### Transfection and Expression of Cross-Over DVD-Ig Molecules in 293 Cells and Characterization of the Cross-Over DVD-Ig

Expression vectors encoding cross-over DVD-Ig molecules capable of binding three to four antigens were constructed using polynucleotides encoding parental monoclonal antibodies specific for antigens A and B respectively. Expression of the reference cross-over DVD-Ig was accomplished by transiently co-transfecting HEK293 (EBNA) cells with plasmids containing the corresponding light-chains (LC) and heavy-chains (HC) nucleic acids. HEK293 (EBNA) cells were propagated in Freestyle 293 media (Invitrogen, Carlsbad Calif.) at a 0.5 L-scale in flasks (2 L Corning Cat#431198) shaking in a CO₂ incubator (8% CO₂, 125 RPM, 37° C.). When the cultures reached a density of 1×10⁶ cells/ml, cells were transfected with transfection complex. Transfection complex was prepared by first mixing 150 μg LC-plasmids and 100 μg HC-plasmids together in 25 ml of Freestyle media, followed by the addition of 500 ul PEI stock solution [stock solution: 1 mg/ml (pH 7.0) Linear 25 kDa PEI, Polysciences Cat#23966]. The transfection complex was mixed by inversion and allowed to incubate at room temperature for 20 minutes prior to being added to the cell culture. Following transfection, cultures continued to be grown in the CO₂ incubator (8% CO₂, 125 RPM, 37° C.). Twenty-four hours after transfection, the culture was supplemented with 25 ml of a 10% Tryptone N1 solution (Organo Technie, La Courneuve France Cat#19553). Nine days after transfection, cells were removed from the cultures by centrifugation (16,000 g, 10 minutes), and the retained supernatant was sterile filtered (Millipore HV Durapore Stericup, 0.45 um) and placed at 4° C. until initiation of the purification step.

Each cross-over DVD-Ig was individually purified using a disposable 2 ml packed column (packed by Orochem Technologies) containing MabSelect SuRe resin (GE Healthcare). Columns were pre-equilibriated in PBS and then loaded with the harvested 0.55 L samples overnight (15 hours) at 1 ml/minute with the flow-through being recirculated back into the feed container. Following the loading step, columns were washed with 20 ml PBS and protein was eluted by feeding elution buffer [50 mM Citric acid pH 3.5] at 4 ml/min and collecting fractions (1 ml) in tubes already containing 0.2 ml of 1.5M Tris pH 8.2 (bringing the final pH to approximately 6.0). Fractions containing antibody were pooled based on the chromatograms and dialyzed into the final storage buffer [10 mM citric acid, 10 mM Na₂HPO₄, pH 6.0]. Following dialysis, samples were filtered through a 0.22 um Steriflip (Millipore) and the protein concentration was determined by absorbance [Hewlett Packard 8453 diode array spectrophotometer]. SDS-PAGE analysis was performed on analytical samples (both reduced and non-reduced) to assess final purity, verify the presence of appropriately sized heavy- and light-chain bands, and confirm the absence of significant amounts of free (e.g., uncomplexed) light chain (in the non-reduced samples).

### Example 6

### Antigen Binding and Protein Express Analysis of Anti-Il-1b/Il-17 Cross-Over DVD-Ig Molecule

The Il-1B and Il-17 binding affinity of selected cross-over DVD-Ig clones was measured by surface plasmon resonsance (Biacore). The ability of the cross-over DVD-Ig clones to neutralize binding of Il-1B and Il-17 to their cognate receptors was also measured and an IC-50 determined. The protein expression levels and percentage monomer produced for each of the selected cross-over DVD-Ig clones was also determined. In each set of experiments, individual Il-1B and Il-17 monoclonal antibodies were used as a benchmark. The results, set forth in Table 6 herein, show that several of the selected cross-over DVD-Ig clones exhibit excellent functional and thermodynamic properties relative to the individual benchmark Il-1B and Il-17 monoclonal antibodies.

### Example 7

### Antigen Binding and Protein Express Analysis of Anti-TNF/Il-17 Cross-Over DVD-Ig Molecules

The variable domains of the B6=17 anti-IL-17 antibody and the hMAK199-AM1 anti-TNF antibody were used to make a full length DVD-Ig™ molecule and two cross-over DVD-Ig™ molcules having format 2 (as disclosed herein). The linker sequences used to make these cross-over DVD-Ig™ molcules are set forth in Table 5 herein, The TNF and Il-17 binding affinity of the DVD-Ig clones was measured by surface plasmon resonsance (Biacore). The ability of the cross-over DVD-Ig clones to neutralize binding of TNF and Il-17 to their cognate receptors was also measured and an IC-50 determined. The protein expression levels and percentage monomer produced for each of the selected cross-over DVD-Ig clones was also determined. In each set of experiments, the individual parental TNF and Il-17 monoclonal antibodies were used as a benchmark. The results, set forth in Table 7 herein, show that several of the cross-over DVD-Ig clones exhibit excellent functional and thermodynamic properties relative to the individual benchmark Il-1B and Il-17 monoclonal antibodies. In particular the cross-over DVD-Ig clones exhibit higher affinity for IL-17 and TNF than both parental antibodies and a higher affinity for IL-17 than the DVD-Ig™ molecule.

TABLE 6
Antigen binding and protein express analysis of anti-Il-1b/Il-17 cross-over DVD-Ig ™ Molecules

| Antibody or | IL1B | IL17 | | | | IL-1B | IL-17 | IL-1B | IL-17 |
|---|---|---|---|---|---|---|---|---|---|
| coDVD-Ig | Binding | Binding | | Expression | % | Biacore kD | Biacore kD | neutralization | neutralization |
| clone | Domain | Domain | Format | (mg/L) | Monomer | (pM) | (pM) | IC50 (pM) | IC50 (pM) |
| AB268 | IL-1B | | mAb | 32 | 99 | 43 | — | 10 | — |
| | (E26.13) | | | | | | | | |
| AB270 | IL-1B | | mAb | 52 | 91 | 89 | — | 245 | — |
| | (1B12.1) | | | | | | | | |
| AB461 | | IL-17 (B6- | mAb | 64 | 93 | NA | 0.9 | NA | 2 |
| | | 5G) | | | | | | | |
| CODH1 | IL-1B | IL17 (B6- | Fomat 2 | 2 | 98 | 190 | <0.1 | 1304 | 30 |
| | (1B12.1) | 5G) | | | | | | | |
| CODH2 | IL-1B | IL17 (B6- | Fomat 2 | 30 | 99 | 170 | 0.4 | 1595 | 29 |
| | (1B12.1) | 5G) | | | | | | | |
| CODH3 | IL-1B | IL17 (B6- | Fomat 2 | 40 | 84 | 130 | <0.2 | 1161 | 12 |
| | (E26.13) | 5G) | | | | | | | |
| CODH4 | IL-1B | IL17 (B6- | Fomat 2 | 2 | 98 | 110 | | 113 | 4 |
| | (E26.13) | 5G) | | | | | | | |
| CODH12 | IL-1B | IL17 (B6- | Fomat 12 | 9 | 98 | 8.0E+05 | No binding | 2044 | 220 |
| | (E26.13) | 5G) | | | | | | | |
| CODH15 | IL-1B | IL17 (B6- | Fomat 11 | 13 | 94 | 3.9E+05 | ND | 1665 | 7.4 |
| | (E26.13) | 5G) | | | | | | | |

TABLE 7
Antigen binding and protein express analysis of anti-TNF/Il-17 cross-over DVD-Ig ™ Molecules

| | | | | | | ELISA | | Neutral- | |
|---|---|---|---|---|---|---|---|---|---|
| | | | | Expres- | | EC50 | | ization | |
| | | | | sion | | (nM) | | IC50 (pM) | |
| Protein | Outer VD | Inner VD | Format | (mg/L) | % Monomer | IL-17 | TNF | IL-17 | TNF |
| AB274 | IL-17 (B6-17) | | mAb | | 99 | 0.203 | — | 10 | — |
| AB441 | TNF (hMAK199-AM1) | | mAb | | 100 | — | 0.398 | — | 6 |
| DVD | TNF(hMAK199- | IL-17 | GS10 | | 98 | 1.385 | 0.216 | 50 | 20 |
| | AM1) | (B6-17) | | | | | | | |

| | | | | | | | | |
|---|---|---|---|---|---|---|---|---|
| CODV 3-36 | TNF(hMAK199-AM1)& IL17 (B6- | 2 | 22 | 98 | 0.087 | 0.189 | TBD | 0.7 |
| | 17) | | | | | | | |
| CODV 44-85 | TNF(hMAK199-AM1)& IL17 (B6- | 2 | 15 | 100 | 0.149 | 0.249 | TBD | 0.4 |
| | 17) | | | | | | | |

### INCORPORATION BY REFERENCE

The contents of all cited references (including literature references, patents, patent applications, and websites) that maybe cited throughout this application are hereby expressly incorporated by reference in their entirety for any purpose, as are the references cited therein. The disclosure will employ, unless otherwise indicated, conventional techniques of immunology, molecular biology and cell biology, which are well known in the art.

The present disclosure also incorporates by reference in their entirety techniques well known in the field of molecular biology and drug delivery. These techniques include, but are not limited to, techniques described in the following publications:
Atwell et al. J. Mol. Biol. 1997, 270: 26-35;
Ausubel et al. (eds.), CURRENT PROTOCOLS IN MOLECULAR BIOLOGY, John Wiley &Sons, NY (1993);
Ausubel, F. M. et al. eds., SHORT PROTOCOLS IN MOLECULAR BIOLOGY (4th Ed. 1999) John Wiley & Sons, NY. (ISBN 0-471-32938-X);
CONTROLLED DRUG BIOAVAILABILITY, DRUG PRODUCT DESIGN AND PERFORMANCE, Smolen and Ball (eds.), Wiley, New York (1984);
Giege, R. and Ducruix, A. Barrett, CRYSTALLIZATION OF NUCLEIC ACIDS AND PROTEINS, a Practical Approach, 2nd ea., pp. 20 1-16, Oxford University Press, New York, N.Y., (1999);
Goodson, in MEDICAL APPLICATIONS OF CONTROLLED RELEASE, vol. 2, pp. 115-138 (1984);
Hammerling, et al., in: MONOCLONAL ANTIBODIES AND T-CELL HYBRIDOMAS 563-681 (Elsevier, N.Y., 1981;
Harlow et al., ANTIBODIES: A LABORATORY MANUAL, (Cold Spring Harbor Laboratory Press, 2nd ed. 1988);
Kabat et al., SEQUENCES OF PROTEINS OF IMMUNOLOGICAL INTEREST (National Institutes of Health, Bethesda, Md. (1987) and (1991);
Kabat, E. A., et al. (1991) SEQUENCES OF PROTEINS OF IMMUNOLOGICAL INTEREST, Fifth Edition, U.S. Department of Health and Human Services, NIH Publication No. 91-3242;
Kontermann and Dubel eds., ANTIBODY ENGINEERING (2001) Springer-Verlag. New York. 790 pp. (ISBN 3-540-41354-5).
Kriegler, Gene Transfer and Expression, A Laboratory Manual, Stockton Press, NY (1990);
Lu and Weiner eds., CLONING AND EXPRESSION VECTORS FOR GENE FUNCTION ANALYSIS (2001) BioTechniques Press. Westborough, Mass. 298 pp. (ISBN 1-881299-21-X).
MEDICAL APPLICATIONS OF CONTROLLED RELEASE, Langer and Wise (eds.), CRC Pres., Boca Raton, Fla. (1974);
Old, R. W. & S. B. Primrose, PRINCIPLES OF GENE MANIPULATION: AN INTRODUCTION TO GENETIC ENGINEERING (3d Ed. 1985) Blackwell Scientific Publications, Boston. Studies in Microbiology; V.2:409 pp. (ISBN 0-632-01318-4).
Sambrook, J. et al. eds., MOLECULAR CLONING: A LABORATORY MANUAL (2d Ed. 1989) Cold Spring Harbor Laboratory Press, NY. Vols. 1-3. (ISBN 0-87969-309-6). SUSTAINED AND CONTROLLED RELEASE DRUG DELIVERY SYSTEMS, J. R. Robinson, ed., Marcel Dekker, Inc., New York, 1978
Winnacker, E. L. FROM GENES TO CLONES: INTRODUCTION TO GENE TECHNOLOGY (1987) VCH Publishers, NY (translated by Horst Ibelgaufts). 634 pp. (ISBN 0-89573-614-4).

### EQUIVALENTS

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

We claim:

1. A binding protein comprising first and second polypeptide chains,
wherein said first polypeptide chain comprises VL1-(X1)n-VL2-CL-(X2)n, wherein VL1 is a first light chain variable domain, VL2 is a second light chain variable domain, CL is a light chain constant domain, X1 is a linker with the proviso that it is not a constant domain, and X2 is an Fc region if X4 is not an Fc region;
wherein said second polypeptide chain comprises VH2-(X3)n-VH1-CH-(X4)n, wherein VH1 is a first heavy chain variable domain, VH2 is a second heavy chain variable domain, CH is a heavy chain constant domain, X1 is a linker with the proviso that it is not a constant domain, and X4 is an Fc region if X2 is not an Fc region;
wherein n is 0 or 1, wherein the VL1 and VH1 domains form one functional binding site for antigen A, and wherein the VL2 and VH2 domains form one functional binding site for antigen B.

2. The binding protein of claim 1, wherein said binding protein is capable of binding both antigens A and B simultaneously.

3. A binding protein comprising first and second polypeptide chains,
wherein said first polypeptide chain comprises VL1-(X1)n-VH2-CL-(X2)n, wherein VL1 is a first light chain variable domain, VH2 is a second heavy chain variable domain, CL is a light chain constant domain, X1 is a linker with the proviso that it is not a constant domain, and X2 is an Fc region if X4 is not an Fc region;
wherein said second polypeptide chain comprises VL2-(X3)n-VH1-CH-(X4)n, wherein VH1 is a first heavy chain variable domain, VL2 is a second light chain variable domain, CH is a heavy chain constant domain, X1 is a linker with the proviso that it is not a constant domain, and X4 is an Fc region if X2 is not an Fc region;
wherein n is 0 or 1, wherein the VL1 and VH1 domains form one functional binding site for antigen A, and wherein the VL2 and VH2 domains form one functional binding site for antigen B.

4. The binding protein of claim 3, wherein said binding protein is capable of binding both antigens A and B simultaneously.


SEQUENCE LISTING (59 sequences)

SEQ ID NO: 1 - protein, 227 aa, Artificial Sequence
  Description of Artificial Sequence Synthetic polypeptide
EIVLTQSPDFQSVTPKEKVTITCRASQNIGSELHWYQQKPDQSPKLLIKYASHSISGVPSRFSGSGSGTDFTLTINGLEAEDAATYYCHQSDTLPHTFGQGTKVDIKGGGGGGGDTQVTQSPSSLSASVGDRVTITCITSTDIDVDMNWYQQKPGKPPKLLISQGNTLRPGVPSRFSSSGSGTDFTFTISSLQPEDFATYYCLQSDNLPLTFGQGTKLEIKGGGGGR

SEQ ID NO: 2 - protein, 251 aa, Artificial Sequence
  Description of Artificial Sequence Synthetic polypeptide
EVQLQESGPGLVKPSETLSLTCTVSGFSLSDYGVSWIRQPPGKGLEWLGLIWGGGDTYYNSPLKSRLTISKDNSKSQVSLKLSSVTAADTAVYYCAKQRTLWGYDLYGMDYWGQGTLVTVSSGEVQLVQSGAEVKKPGESVKISCKASGGSFRSYGISWVRQAPGQGLEWMGGITHFFGITDYAQKFQGRVTITADESTTTAYMELSGLTSDDTAVYYCAREPNDFWGGYYDTHDFDSWGQGTTVTVSSGG

SEQ ID NO: 3 - protein, 227 aa, Artificial Sequence
  Description of Artificial Sequence Synthetic polypeptide
DTQVTQSPSSLSASVGDRVTITCITSTDIDVDMNWYQQKPGKPPKLLISQGNTLRPGVPSRFSSSGSGTDFTFTISSLQPEDFATYYCLQSDNLPLTFGQGTKLEIKGGGGGGGEIVLTQSPDFQSVTPKEKVTITCRASQNIGSELHWYQQKPDQSPKLLIKYASHSISGVPSRFSGSGSGTDFTLTINGLEAEDAATYYCHQSDTLPHTFGQGTKVDIKGGGGGR

SEQ ID NO: 4 - protein, 251 aa, Artificial Sequence
  Description of Artificial Sequence Synthetic polypeptide
EVQLVQSGAEVKKPGESVKISCKASGGSFRSYGISWVRQAPGQGLEWMGGITHFFGITDYAQKFQGRVTITADESTTTAYMELSGLTSDDTAVYYCAREPNDFWGGYYDTHDFDSWGQGTTVTVSSGEVQLQESGPGLVKPSETLSLTCTVSGFSLSDYGVSWIRQPPGKGLEWLGLIWGGGDTYYNSPLKSRLTISKDNSKSQVSLKLSSVTAADTAVYYCAKQRTLWGYDLYGMDYWGQGTLVTVSSGG

SEQ ID NO: 5 - protein, 227 aa, Artificial Sequence
  Description of Artificial Sequence Synthetic polypeptide
EIVLTQSPDFQSVTPKEKVTITCRASQNIGSELHWYQQKPDQSPKLLIKYASHSISGVPSRFSGSGSGTDFTLTINGLEAEDAATYYCHQSDTLPHTFGQGTKVDIKGGGGGGGDIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLQITGGGGGR

SEQ ID NO: 6 - protein, 248 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTPVTVSSGEVQLVQSGAEVKKPGESVKISCKASGGSFRSYGISWVRQAPGQGLEWMGGITHFFGITDYAQKFQGRVTITADESTTTAYMELSGLTSDDTAVYYCAREPNDFWGGYYDTHDFDSWGQGTTVTVSSGG

SEQ ID NO: 7 - protein, 227 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLQITGGGGGGGEIVLTQSPDFQSVTPKEKVTITCRASQNIGSELHWYQQKPDQSPKLLIKYASHSISGVPSRFSGSGSGTDFTLTINGLEAEDAATYYCHQSDTLPHTFGQGTKVDIKGGGGGR

SEQ ID NO: 8 - protein, 248 aa, Artificial Sequence
  Description of Artificial Sequence Synthetic polypeptide
EVQLVQSGAEVKKPGESVKISCKASGGSFRSYGISWVRQAPGQGLEWMGGITHFFGITDYAQKFQGRVTITADESTTTAYMELSGLTSDDTAVYYCAREPNDFWGGYYDTHDFDSWGQGTTVTVSSGEVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTPVTVSSGG

SEQ ID NO: 9 - protein, 250 aa, Artificial Sequence
  Description of Artificial Sequence Synthetic polypeptide
DTQVTQSPSSLSASVGDRVTITCITSTDIDVDMNWYQQKPGKPPKLLISQGNTLRPGVPSRFSSSGSGTDFTFTISSLQPEDFATYYCLQSDNLPLTFGQGTKLEIKGGGSGGGGEVQLVQSGAEVKKPGESVKISCKASGGSFRSYGISWVRQAPGQGLEWMGGITHFFGITDYAQKFQGRVTITADESTTTAYMELSGLTSDDTAVYYCAREPNDFWGGYYDTHDFDSWGQGTTVTVSSLGGCGGGSR

SEQ ID NO: 10 - protein, 245 aa, Artificial Sequence
  Description of Artificial Sequence Synthetic polypeptide
EIVLTQSPDFQSVTPKEKVTITCRASQNIGSELHWYQQKPDQSPKLLIKYASHSISGVPSRFSGSGSGTDFTLTINGLEAEDAATYYCHQSDTLPHTFGQGTKVDIKGGGSGGGGEVQLQESGPGLVKPSETLSLTCTVSGFSLSDYGVSWIRQPPGKGLEWLGLIWGGGDTYYNSPLKSRLTISKDNSKSQVSLKLSSVTAADTAVYYCAKQRTLWGYDLYGMDYWGQGTLVTVSSLGGCGGGS

SEQ ID NO: 11 - protein, 246 aa, Artificial Sequence
  Description of Artificial Sequence Synthetic polypeptide
EIVLTQSPDFQSVTPKEKVTITCRASQNIGSELHWYQQKPDQSPKLLIKYASHSISGVPSRFSGSGSGTDFTLTINGLEAEDAATYYCHQSDTLPHTFGQGTKVDIKGGGSGGGGEVQLQESGPGLVKPSETLSLTCTVSGFSLSDYGVSWIRQPPGKGLEWLGLIWGGGDTYYNSPLKSRLTISKDNSKSQVSLKLSSVTAADTAVYYCAKQRTLWGYDLYGMDYWGQGTLVTVSSLGGCGGGSR

SEQ ID NO: 12 - protein, 249 aa, Artificial Sequence
  Description of Artificial Sequence Synthetic polypeptide
DTQVTQSPSSLSASVGDRVTITCITSTDIDVDMNWYQQKPGKPPKLLISQGNTLRPGVPSRFSSSGSGTDFTFTISSLQPEDFATYYCLQSDNLPLTFGQGTKLEIKGGGSGGGGEVQLVQSGAEVKKPGESVKISCKASGGSFRSYGISWVRQAPGQGLEWMGGITHFFGITDYAQKFQGRVTITADESTTTAYMELSGLTSDDTAVYYCAREPNDFWGGYYDTHDFDSWGQGTTVTVSSLGGCGGGS

SEQ ID NO: 13 - protein, 250 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLQITGGGSGGGGEVQLVQSGAEVKKPGESVKISCKASGGSFRSYGISWVRQAPGQGLEWMGGITHFFGITDYAQKFQGRVTITADESTTTAYMELSGLTSDDTAVYYCAREPNDFWGGYYDTHDFDSWGQGTTVTVSSLGGCGGGSR

SEQ ID NO: 14 - protein, 242 aa, Artificial Sequence
  Description of Artificial Sequence Synthetic polypeptide
EIVLTQSPDFQSVTPKEKVTITCRASQNIGSELHWYQQKPDQSPKLLIKYASHSISGVPSRFSGSGSGTDFTLTINGLEAEDAATYYCHQSDTLPHTFGQGTKVDIKGGGSGGGGEVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTPVTVSSLGGCGGGS

SEQ ID NO: 15 - protein, 243 aa, Artificial Sequence
  Description of Artificial Sequence Synthetic polypeptide
EIVLTQSPDFQSVTPKEKVTITCRASQNIGSELHWYQQKPDQSPKLLIKYASHSISGVPSRFSGSGSGTDFTLTINGLEAEDAATYYCHQSDTLPHTFGQGTKVDIKGGGSGGGGEVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTPVTVSSLGGCGGGSR

SEQ ID NO: 16 - protein, 249 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLQITGGGSGGGGEVQLVQSGAEVKKPGESVKISCKASGGSFRSYGISWVRQAPGQGLEWMGGITHFFGITDYAQKFQGRVTITADESTTTAYMELSGLTSDDTAVYYCAREPNDFWGGYYDTHDFDSWGQGTTVTVSSLGGCGGGS

SEQ ID NO: 17 - protein, 241 aa, Artificial Sequence
  Description of Artificial Sequence Synthetic polypeptide
EVQLQESGPGLVKPSETLSLTCTVSGFSLSDYGVSWIRQPPGKGLEWLGLIWGGGDTYYNSPLKSRLTISKDNSKSQVSLKLSSVTAADTAVYYCAKQRTLWGYDLYGMDYWGQGTLVTVSSGGGGSGGGEIVLTQSPDFQSVTPKEKVTITCRASQNIGSELHWYQQKPDQSPKLLIKYASHSISGVPSRFSGSGSGTDFTLTINGLEAEDAATYYCHQSDTLPHTFGQGTKVDIKGGGR

SEQ ID NO: 18 - protein, 246 aa, Artificial Sequence
  Description of Artificial Sequence Synthetic polypeptide
EVQLVQSGAEVKKPGESVKISCKASGGSFRSYGISWVRQAPGQGLEWMGGITHFFGITDYAQKFQGRVTITADESTTTAYMELSGLTSDDTAVYYCAREPNDFWGGYYDTHDFDSWGQGTTVTVSSGGGGSGGGDTQVTQSPSSLSASVGDRVTITCITSTDIDVDMNWYQQKPGKPPKLLISQGNTLRPGVPSRFSSSGSGTDFTFTISSLQPEDFATYYCLQSDNLPLTFGQGTKLEIKGGGSG

SEQ ID NO: 19 - protein, 245 aa, Artificial Sequence
  Description of Artificial Sequence Synthetic polypeptide
EVQLVQSGAEVKKPGESVKISCKASGGSFRSYGISWVRQAPGQGLEWMGGITHFFGITDYAQKFQGRVTITADESTTTAYMELSGLTSDDTAVYYCAREPNDFWGGYYDTHDFDSWGQGTTVTVSSGGGGSGGGDTQVTQSPSSLSASVGDRVTITCITSTDIDVDMNWYQQKPGKPPKLLISQGNTLRPGVPSRFSSSGSGTDFTFTISSLQPEDFATYYCLQSDNLPLTFGQGTKLEIKGGGR

SEQ ID NO: 20 - protein, 242 aa, Artificial Sequence
  Description of Artificial Sequence Synthetic polypeptide
EVQLQESGPGLVKPSETLSLTCTVSGFSLSDYGVSWIRQPPGKGLEWLGLIWGGGDTYYNSPLKSRLTISKDNSKSQVSLKLSSVTAADTAVYYCAKQRTLWGYDLYGMDYWGQGTLVTVSSGGGGSGGGEIVLTQSPDFQSVTPKEKVTITCRASQNIGSELHWYQQKPDQSPKLLIKYASHSISGVPSRFSGSGSGTDFTLTINGLEAEDAATYYCHQSDTLPHTFGQGTKVDIKGGGSG

SEQ ID NO: 21 - protein, 238 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTPVTVSSGGGGSGGGEIVLTQSPDFQSVTPKEKVTITCRASQNIGSELHWYQQKPDQSPKLLIKYASHSISGVPSRFSGSGSGTDFTLTINGLEAEDAATYYCHQSDTLPHTFGQGTKVDIKGGGR

SEQ ID NO: 22 - protein, 246 aa, Artificial Sequence
  Description of Artificial Sequence Synthetic polypeptide
EVQLVQSGAEVKKPGESVKISCKASGGSFRSYGISWVRQAPGQGLEWMGGITHFFGITDYAQKFQGRVTITADESTTTAYMELSGLTSDDTAVYYCAREPNDFWGGYYDTHDFDSWGQGTTVTVSSGGGGSGGGDIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLQITGGSGG

SEQ ID NO: 23 - protein, 245 aa, Artificial Sequence
  Description of Artificial Sequence Synthetic polypeptide
EVQLVQSGAEVKKPGESVKISCKASGGSFRSYGISWVRQAPGQGLEWMGGITHFFGITDYAQKFQGRVTITADESTTTAYMELSGLTSDDTAVYYCAREPNDFWGGYYDTHDFDSWGQGTTVTVSSGGGGSGGGDIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLQITGGGR

SEQ ID NO: 24 - protein, 239 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTPVTVSSGGGGSGGGEIVLTQSPDFQSVTPKEKVTITCRASQNIGSELHWYQQKPDQSPKLLIKYASHSISGVPSRFSGSGSGTDFTLTINGLEAEDAATYYCHQSDTLPHTFGQGTKVDIKGGSGG

SEQ ID NO: 25 - protein, 252 aa, Artificial Sequence
  Description of Artificial Sequence Synthetic polypeptide
EVQLQESGPGLVKPSETLSLTCTVSGFSLSDYGVSWIRQPPGKGLEWLGLIWGGGDTYYNSPLKSRLTISKDNSKSQVSLKLSSVTAADTAVYYCAKQRTLWGYDLYGMDYWGQGTLVTVSSGEVQLVQSGAEVKKPGESVKISCKASGGSFRSYGISWVRQAPGQGLEWMGGITHFFGITDYAQKFQGRVTITADESTTTAYMELSGLTSDDTAVYYCAREPNDFWGGYYDTHDFDSWGQGTTVTVSSGGR

SEQ ID NO: 26 - protein, 226 aa, Artificial Sequence
  Description of Artificial Sequence Synthetic polypeptide
EIVLTQSPDFQSVTPKEKVTITCRASQNIGSELHWYQQKPDQSPKLLIKYASHSISGVPSRFSGSGSGTDFTLTINGLEAEDAATYYCHQSDTLPHTFGQGTKVDIKGGGGGGGDTQVTQSPSSLSASVGDRVTITCITSTDIDVDMNWYQQKPGKPPKLLISQGNTLRPGVPSRFSSSGSGTDFTFTISSLQPEDFATYYCLQSDNLPLTFGQGTKLEIKGGGGG

SEQ ID NO: 27 - protein, 252 aa, Artificial Sequence
  Description of Artificial Sequence Synthetic polypeptide
EVQLVQSGAEVKKPGESVKISCKASGGSFRSYGISWVRQAPGQGLEWMGGITHFFGITDYAQKFQGRVTITADESTTTAYMELSGLTSDDTAVYYCAREPNDFWGGYYDTHDFDSWGQGTTVTVSSGEVQLQESGPGLVKPSETLSLTCTVSGFSLSDYGVSWIRQPPGKGLEWLGLIWGGGDTYYNSPLKSRLTISKDNSKSQVSLKLSSVTAADTAVYYCAKQRTLWGYDLYGMDYWGQGTLVTVSSGGR

SEQ ID NO: 28 - protein, 226 aa, Artificial Sequence
  Description of Artificial Sequence Synthetic polypeptide
DTQVTQSPSSLSASVGDRVTITCITSTDIDVDMNWYQQKPGKPPKLLISQGNTLRPGVPSRFSSSGSGTDFTFTISSLQPEDFATYYCLQSDNLPLTFGQGTKLEIKGGGGGGGEIVLTQSPDFQSVTPKEKVTITCRASQNIGSELHWYQQKPDQSPKLLIKYASHSISGVPSRFSGSGSGTDFTLTINGLEAEDAATYYCHQSDTLPHTFGQGTKVDIKGGGGG

SEQ ID NO: 29 - protein, 249 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTPVTVSSGEVQLVQSGAEVKKPGESVKISCKASGGSFRSYGISWVRQAPGQGLEWMGGITHFFGITDYAQKFQGRVTITADESTTTAYMELSGLTSDDTAVYYCAREPNDFWGGYYDTHDFDSWGQGTTVTVSSGGR

SEQ ID NO: 30 - protein, 226 aa, Artificial Sequence
  Description of Artificial Sequence Synthetic polypeptide
EIVLTQSPDFQSVTPKEKVTITCRASQNIGSELHWYQQKPDQSPKLLIKYASHSISGVPSRFSGSGSGTDFTLTINGLEAEDAATYYCHQSDTLPHTFGQGTKVDIKGGGGGGGDIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLQITGGGGG

SEQ ID NO: 31 - protein, 249 aa, Artificial Sequence
  Description of Artificial Sequence Synthetic polypeptide
EVQLVQSGAEVKKPGESVKISCKASGGSFRSYGISWVRQAPGQGLEWMGGITHFFGITDYAQKFQGRVTITADESTTTAYMELSGLTSDDTAVYYCAREPNDFWGGYYDTHDFDSWGQGTTVTVSSGEVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTPVTVSSGGR

SEQ ID NO: 32 - protein, 226 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLQITGGGGGGGEIVLTQSPDFQSVTPKEKVTITCRASQNIGSELHWYQQKPDQSPKLLIKYASHSISGVPSRFSGSGSGTDFTLTINGLEAEDAATYYCHQSDTLPHTFGQGTKVDIKGGGGG

SEQ ID NO: 33 - protein, 4 aa, Artificial Sequence
  Description of Artificial Sequence Synthetic peptide
GGGS

SEQ ID NO: 34 - protein, 5 aa, Artificial Sequence
  Description of Artificial Sequence Synthetic peptide
GGGGS

SEQ ID NO: 35 - protein, 5 aa, Artificial Sequence
  Description of Artificial Sequence Synthetic peptide
GGGSG

SEQ ID NO: 36 - protein, 7 aa, Artificial Sequence
  Description of Artificial Sequence Synthetic peptide
GGGSGGG

SEQ ID NO: 37 - protein, 8 aa, Artificial Sequence
  Description of Artificial Sequence Synthetic peptide
GGGGSGGG

SEQ ID NO: 38 - protein, 8 aa, Artificial Sequence
  Description of Artificial Sequence Synthetic peptide
LGGCGGGS

SEQ ID NO: 39 - protein, 10 aa, Artificial Sequence
  Description of Artificial Sequence Synthetic peptide
GGGGSGGGGS

SEQ ID NO: 40 - protein, 8 aa, Artificial Sequence
  Description of Artificial Sequence Synthetic peptide
GGGSGGGG

SEQ ID NO: 41 - protein, 7 aa, Artificial Sequence
  Description of Artificial Sequence Synthetic peptide
GGGGGGG

SEQ ID NO: 42 - protein, 5 aa, Artificial Sequence
  Description of Artificial Sequence Synthetic peptide
GGGGG

SEQ ID NO: 43 - protein, 5 aa, Artificial Sequence
  Description of Artificial Sequence Synthetic peptide
GGSGG

SEQ ID NO: 44 - protein, 227 aa, Artificial Sequence
  Description of Artificial Sequence Synthetic polypeptide
EIVLTQSPDFQSVTPKEKVTITCRASQNIGSELHWYQQKPDQSPKLLIKYASHSISGVPSRFSGSGSGTDFTLTINGLEAEDAATYYCHQSDTLPHTFGQGTKVDIKGGGSGGGDIQMTQSPSSLSASVGDRVTITCQASTDIDDDLNWYQQKPGKAPKLLISLGSTLRPGVPSRFSGSGSGTDFTFTISSLQPEDFATYYCLQSDRLPLTFGQGTKLEIKGGGSGR

SEQ ID NO: 45 - protein, 251 aa, Artificial Sequence
  Description of Artificial Sequence Synthetic polypeptide
EVQLQESGPGLVKPSETLSLTCTVSGFSLSDYGVSWIRQPPGKGLEWLGLIWGGGDTYYNSPLKSRLTISKDNSKSQVSLKLSSVTAADTAVYYCARQTNLWAYDLYSMDYWGQGTLVTVSSGEVQLVQSGAEVKKPGESVKISCKASGGSFRSYGISWVRQAPGQGLEWMGGITHFFGITDYAQKFQGRVTITADESTTTAYMELSGLTSDDTAVYYCAREPNDFWGGYYDTHDFDSWGQGTTVTVSSGG

SEQ ID NO: 46 - protein, 227 aa, Artificial Sequence
  Description of Artificial Sequence Synthetic polypeptide
DIQMTQSPSSLSASVGDRVTITCQASTDIDDDLNWYQQKPGKAPKLLISLGSTLRPGVPSRFSGSGSGTDFTFTISSLQPEDFATYYCLQSDRLPLTFGQGTKLEIKGGGSGGGEIVLTQSPDFQSVTPKEKVTITCRASQNIGSELHWYQQKPDQSPKLLIKYASHSISGVPSRFSGSGSGTDFTLTINGLEAEDAATYYCHQSDTLPHTFGQGTKVDIKGGGSGR

SEQ ID NO: 47 - protein, 251 aa, Artificial Sequence
  Description of Artificial Sequence Synthetic polypeptide
EVQLVQSGAEVKKPGESVKISCKASGGSFRSYGISWVRQAPGQGLEWMGGITHFFGITDYAQKFQGRVTITADESTTTAYMELSGLTSDDTAVYYCAREPNDFWGGYYDTHDFDSWGQGTTVTVSSGEVQLQESGPGLVKPSETLSLTCTVSGFSLSDYGVSWIRQPPGKGLEWLGLIWGGGDTYYNSPLKSRLTISKDNSKSQVSLKLSSVTAADTAVYYCARQTNLWAYDLYSMDYWGQGTLVTVSSGG

SEQ ID NO: 48 - protein, 250 aa, Artificial Sequence
  Description of Artificial Sequence Synthetic polypeptide
DIQMTQSPSSLSASVGDRVTITCQASTDIDDDLNWYQQKPGKAPKLLISLGSTLRPGVPSRFSGSGSGTDFTFTISSLQPEDFATYYCLQSDRLPLTFGQGTKLEIKGGGSGGGGEVQLVQSGAEVKKPGESVKISCKASGGSFRSYGISWVRQAPGQGLEWMGGITHFFGITDYAQKFQGRVTITADESTTTAYMELSGLTSDDTAVYYCAREPNDFWGGYYDTHDFDSWGQGTTVTVSSLGGCGGGSR

SEQ ID NO: 49 - protein, 245 aa, Artificial Sequence
  Description of Artificial Sequence Synthetic polypeptide
EIVLTQSPDFQSVTPKEKVTITCRASQNIGSELHWYQQKPDQSPKLLIKYASHSISGVPSRFSGSGSGTDFTLTINGLEAEDAATYYCHQSDTLPHTFGQGTKVDIKGGGSGGGGEVQLQESGPGLVKPSETLSLTCTVSGFSLSDYGVSWIRQPPGKGLEWLGLIWGGGDTYYNSPLKSRLTISKDNSKSQVSLKLSSVTAADTAVYYCARQTNLWAYDLYSMDYWGQGTLVTVSSLGGCGGGS

SEQ ID NO: 50 - protein, 246 aa, Artificial Sequence
  Description of Artificial Sequence Synthetic polypeptide
EIVLTQSPDFQSVTPKEKVTITCRASQNIGSELHWYQQKPDQSPKLLIKYASHSISGVPSRFSGSGSGTDFTLTINGLEAEDAATYYCHQSDTLPHTFGQGTKVDIKGGGSGGGGEVQLQESGPGLVKPSETLSLTCTVSGFSLSDYGVSWIRQPPGKGLEWLGLIWGGGDTYYNSPLKSRLTISKDNSKSQVSLKLSSVTAADTAVYYCARQTNLWAYDLYSMDYWGQGTLVTVSSLGGCGGGSR

SEQ ID NO: 51 - protein, 249 aa, Artificial Sequence
  Description of Artificial Sequence Synthetic polypeptide
DIQMTQSPSSLSASVGDRVTITCQASTDIDDDLNWYQQKPGKAPKLLISLGSTLRPGVPSRFSGSGSGTDFTFTISSLQPEDFATYYCLQSDRLPLTFGQGTKLEIKGGGSGGGGEVQLVQSGAEVKKPGESVKISCKASGGSFRSYGISWVRQAPGQGLEWMGGITHFFGITDYAQKFQGRVTITADESTTTAYMELSGLTSDDTAVYYCAREPNDFWGGYYDTHDFDSWGQGTTVTVSSLGGCGGGS

SEQ ID NO: 52 - protein, 241 aa, Artificial Sequence
  Description of Artificial Sequence Synthetic polypeptide
EVQLQESGPGLVKPSETLSLTCTVSGFSLSDYGVSWIRQPPGKGLEWLGLIWGGGDTYYNSPLKSRLTISKDNSKSQVSLKLSSVTAADTAVYYCARQTNLWAYDLYSMDYWGQGTLVTVSSGGGGSGGGEIVLTQSPDFQSVTPKEKVTITCRASQNIGSELHWYQQKPDQSPKLLIKYASHSISGVPSRFSGSGSGTDFTLTINGLEAEDAATYYCHQSDTLPHTFGQGTKVDIKGGGR

SEQ ID NO: 53 - protein, 246 aa, Artificial Sequence
  Description of Artificial Sequence Synthetic polypeptide
EVQLVQSGAEVKKPGESVKISCKASGGSFRSYGISWVRQAPGQGLEWMGGITHFFGITDYAQKFQGRVTITADESTTTAYMELSGLTSDDTAVYYCAREPNDFWGGYYDTHDFDSWGQGTTVTVSSGGGGSGGGDIQMTQSPSSLSASVGDRVTITCQASTDIDDDLNWYQQKPGKAPKLLISLGSTLRPGVPSRFSGSGSGTDFTFTISSLQPEDFATYYCLQSDRLPLTFGQGTKLEIKGGGSG

SEQ ID NO: 54 - protein, 245 aa, Artificial Sequence
  Description of Artificial Sequence Synthetic polypeptide
EVQLVQSGAEVKKPGESVKISCKASGGSFRSYGISWVRQAPGQGLEWMGGITHFFGITDYAQKFQGRVTITADESTTTAYMELSGLTSDDTAVYYCAREPNDFWGGYYDTHDFDSWGQGTTVTVSSGGGGSGGGDIQMTQSPSSLSASVGDRVTITCQASTDIDDDLNWYQQKPGKAPKLLISLGSTLRPGVPSRFSGSGSGTDFTFTISSLQPEDFATYYCLQSDRLPLTFGQGTKLEIKGGGR

SEQ ID NO: 55 - protein, 242 aa, Artificial Sequence
  Description of Artificial Sequence Synthetic polypeptide
EVQLQESGPGLVKPSETLSLTCTVSGFSLSDYGVSWIRQPPGKGLEWLGLIWGGGDTYYNSPLKSRLTISKDNSKSQVSLKLSSVTAADTAVYYCARQTNLWAYDLYSMDYWGQGTLVTVSSGGGGSGGGEIVLTQSPDFQSVTPKEKVTITCRASQNIGSELHWYQQKPDQSPKLLIKYASHSISGVPSRFSGSGSGTDFTLTINGLEAEDAATYYCHQSDTLPHTFGQGTKVDIKGGGSG

SEQ ID NO: 56 - protein, 252 aa, Artificial Sequence
  Description of Artificial Sequence Synthetic polypeptide
EVQLQESGPGLVKPSETLSLTCTVSGFSLSDYGVSWIRQPPGKGLEWLGLIWGGGDTYYNSPLKSRLTISKDNSKSQVSLKLSSVTAADTAVYYCARQTNLWAYDLYSMDYWGQGTLVTVSSGEVQLVQSGAEVKKPGESVKISCKASGGSFRSYGISWVRQAPGQGLEWMGGITHFFGITDYAQKFQGRVTITADESTTTAYMELSGLTSDDTAVYYCAREPNDFWGGYYDTHDFDSWGQGTTVTVSSGGR

SEQ ID NO: 57 - protein, 226 aa, Artificial Sequence
  Description of Artificial Sequence Synthetic polypeptide
EIVLTQSPDFQSVTPKEKVTITCRASQNIGSELHWYQQKPDQSPKLLIKYASHSISGVPSRFSGSGSGTDFTLTINGLEAEDAATYYCHQSDTLPHTFGQGTKVDIKGGGSGGGDIQMTQSPSSLSASVGDRVTITCQASTDIDDDLNWYQQKPGKAPKLLISLGSTLRPGVPSRFSGSGSGTDFTFTISSLQPEDFATYYCLQSDRLPLTFGQGTKLEIKGGGSG

SEQ ID NO: 58 - protein, 252 aa, Artificial Sequence
  Description of Artificial Sequence Synthetic polypeptide
EVQLVQSGAEVKKPGESVKISCKASGGSFRSYGISWVRQAPGQGLEWMGGITHFFGITDYAQKFQGRVTITADESTTTAYMELSGLTSDDTAVYYCAREPNDFWGGYYDTHDFDSWGQGTTVTVSSGEVQLQESGPGLVKPSETLSLTCTVSGFSLSDYGVSWIRQPPGKGLEWLGLIWGGGDTYYNSPLKSRLTISKDNSKSQVSLKLSSVTAADTAVYYCARQTNLWAYDLYSMDYWGQGTLVTVSSGGR

SEQ ID NO: 59 - protein, 226 aa, Artificial Sequence
  Description of Artificial Sequence Synthetic polypeptide
DIQMTQSPSSLSASVGDRVTITCQASTDIDDDLNWYQQKPGKAPKLLISLGSTLRPGVPSRFSGSGSGTDFTFTISSLQPEDFATYYCLQSDRLPLTFGQGTKLEIKGGGSGGGEIVLTQSPDFQSVTPKEKVTITCRASQNIGSELHWYQQKPDQSPKLLIKYASHSISGVPSRFSGSGSGTDFTLTINGLEAEDAATYYCHQSDTLPHTFGQGTKVDIKGGGSG